SEQUENCE LISTING (109 sequences)

SEQ ID NO: 1 - protein, 125 aa, Artificial Sequence
  Synthetic Polypeptide
GYQGPPGPPGPPGIPGNHGNNGNNGATGHEGAKGEKGDKGDLGPRGERGQHGPKGEKGYPGIPGYCGPGPPGPPGIPGNHGNNGNNGATGHEGAKGEKGDKGDLGPRGERGQHGPKGEKGYPGIP

SEQ ID NO: 2 - protein, 126 aa, Artificial Sequence
  Synthetic Polypeptide
GEKGDRGDRGLQGKYGKTGSAGARGHTGPKGQKGSMGAPGERGEKGDRGDRGLQGKYGKTGSAGARGHTGPKGQKGSMGAPGERGEKGDRGDRGLQGKYGKTGSAGARGHTGPKGQKGSMGAPGER

SEQ ID NO: 3 - protein, 102 aa, Artificial Sequence
  Synthetic Polypeptide
GPQGPPGPPGAPGPSGMMGRMGFPGKDGQDGHDGDRGDSGEEGPPGRTGNRGKPGPKGKAGAIGRAGPRGPKGVNGTPGKHGTPGKKGPKGKKGEPGLPGPC

SEQ ID NO: 4 - protein, 138 aa, Artificial Sequence
  Synthetic Polypeptide
GHPGLPGTPGHHGSQGLPGRDGRDGRDGAPGAPGEKGEGGRPGLPGPRGDPGPRGEAGPAGPTGPAGECGHPGLPGTPGHHGSQGLPGRDGRDGRDGAPGAPGEKGEGGRPGLPGPRGDPGPRGEAGPAGPTGPAGEC

SEQ ID NO: 5 - protein, 126 aa, Artificial Sequence
  Synthetic Polypeptide
GDKGDPGPMGLPGYMGREGPQGEPGPQGSKGDKGEMGSPGAPGDKGDPGPMGLPGYMGREGPQGEPGPQGSKGDKGEMGSPGAPGDKGDPGPMGLPGYMGREGPQGEPGPQGSKGDKGEMGSPGAP

SEQ ID NO: 6 - protein, 107 aa, Artificial Sequence
  Synthetic Polypeptide
GLPGPPGPPGANGSPGPHGRIGLPGRDGRDGRKGEKGEKGTAGLRGKTGPLGLAGEKGDQGETGKKGPIGPEGEKGEVGPIGPPGPKGDRGEQGDPGLPGVCRCGSI

SEQ ID NO: 7 - protein, 126 aa, Artificial Sequence
  Synthetic Polypeptide
GEKGEAGVRGRAGRSGKEGPPGARGLQGRRGQKGQVGPPGAAGEKGEAGVRGRAGRSGKEGPPGARGLQGRRGQKGQVGPPGAAGEKGEAGVRGRAGRSGKEGPPGARGLQGRRGQKGQVGPPGAA

SEQ ID NO: 8 - protein, 171 aa, Artificial Sequence
  Synthetic Polypeptide
GHPGIPGNPGHNGLPGRDGRDGAKGDKGDAGEPGRPGSPGKDGTSGEKGERGADGKVEAKGIKGDQGSRGSPGKHGPKGLAGPMGEKGLRGETGPQGQKGNKGDVGPTGPEGPRGNIGPLGPTGLPGPMGPIGKPGPKGEAGPTGPQGEPGVRGIRGWKGDRGEKGKIGET

SEQ ID NO: 9 - protein, 171 aa, Artificial Sequence
  Synthetic Polypeptide
GHPGIPGNPGHNGLPGRDGRDGAKGDKGDAGEPGCPGSPGKDGTSGEKGERGADGKVEAKGIKGDQGSRGSPGKHGPKGLAGPMGEKGLRGETGPQGQKGNKGDVGPTGPEGPRGNIGPLGPTGLPGPMGPIGKPGPKGEAGPTGPQGEPGVRGIRGWKGDRGEKGKIGET

SEQ ID NO: 10 - protein, 63 aa, Artificial Sequence
  Synthetic Polypeptide
GYQGPPGPPGPPGIPGNHGNNGNNGATGHEGAKGEKGDKGDLGPRGERGQHGPKGEKGYPGIP

SEQ ID NO: 11 - protein, 146 aa, Artificial Sequence
  Synthetic Polypeptide
MGYQGPPGPPGPPGIPGNHGNNGNNGATGHEGAKGEKGDKGDLGPRGERGQHGPKGEKGYPGIPGYCGPGPPGPPGIPGNHGNNGNNGATGHEGAKGEKGDKGDLGPRGERGQHGPKGEKGYPGIPDYKDDDDKLQISSTVAAARV

SEQ ID NO: 12 - protein, 8 aa, Artificial Sequence
  Synthetic Polypeptide
DYKDDDDK

SEQ ID NO: 13 - protein, 378 aa, Artificial Sequence
  Synthetic Polypeptide
METDTLLLWVLLLWVPGSTGDLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLAAAHHHHHHV

SEQ ID NO: 14 - protein, 316 aa, Artificial Sequence
  Synthetic Polypeptide
METDTLLLWVLLLWVPGSTGDLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISHPEPHTAELQLNLTVPRKDPTLRWGAGPALGRSFTHGPELEEGHLRIHQDGLYRLHIQVTLANCSSPGSTLQHRATLAVGICSPAAHGISLLRGRFGQDCTVALQRLTYLVHGDVLCTNLTLPLLPSRNADETFFGVQWICPAAAHHHHHHV

SEQ ID NO: 15 - protein, 316 aa, Artificial Sequence; identical to SEQ ID NO: 14
  Synthetic Polypeptide

SEQ ID NO: 16 - protein, 371 aa, Artificial Sequence
  Synthetic Polypeptide
METDTLLLWVLLLWVPGSTGDLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWEAAAHHHHHHV

SEQ ID NO: 17 - protein, 345 aa, Artificial Sequence
  Synthetic Polypeptide
METDTLLLWVLLLWVPGSTGDLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISHLPDGGKGSWEKLIQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMVAAAHHHHHHV

SEQ ID NO: 18 - protein, 313 aa, Artificial Sequence
  Synthetic Polypeptide
METDTLLLWVLLLWVPGSTGDLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPLAAAHHHHHHV

SEQ ID NO: 19 - protein, 395 aa, Artificial Sequence
  Synthetic Polypeptide
MLRGWGGPSVGVCVRTALGVLCLCLTGAVEVQVSEDPVVALVDTDATLRCSFSPEPGFSLAQLNLIWQLTDTKQLVHSFTEGRDQGSAYSNRTALFPDLLVQGNASLRLQRVRVTDEGSYTCFVSIQDFDSAAVSLQVAAPYSKPSMTLEPNKDLRPGNMVTITCSSYQGYPEAEVFWKDGQGVPLTGNVTTSQMANERGLFDVHSVLRVVLGANGTYSCLVRNPVLQQDAHGSVTITGLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 20 - protein, 404 aa, Artificial Sequence
  Synthetic Polypeptide
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 21 - protein, 330 aa, Artificial Sequence
  Synthetic Polypeptide
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEELKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 22 - protein, 329 aa, Artificial Sequence
  Synthetic Polypeptide
MAWPPPYWLCMLGTLVGLSATLAPNSCPDKHYWTGGGLCCRMCEPGTFFVKDCEQDRTAAQCDPCIPGTSFSPDYHTRPHCESCRHCNSGFLIRNCTVTANAECSCSKNWQCRDQECTECDPPLNPALTRQPSETPSPQPPPTHLPHGTEKPSWPLHRQLPNSTVYSQRSSHRLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 23 - protein, 297 aa, Artificial Sequence
  Synthetic Polypeptide
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 24 - protein, 340 aa, Artificial Sequence
  Synthetic Polypeptide
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 25 - protein, 393 aa, Artificial Sequence
  Synthetic Polypeptide
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 26 - protein, 391 aa, Artificial Sequence
  Synthetic Polypeptide
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLDQNVTGNSGINLTCMSKQGHPKPKKMYFLITNSTNEYGDNMQISQDNVTELFSISNSLSLSFPDGVWHMTVVCVLETESMKISSKPLNFTQELKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 27 - protein, 334 aa, Artificial Sequence
  Synthetic Polypeptide
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 28 - protein, 308 aa, Artificial Sequence
  Synthetic Polypeptide
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVILKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 29 - protein, 340 aa, Artificial Sequence
  Synthetic Polypeptide
MFSGLTLNCVLLLLQLLLARSLEDGYKVEVGKNAYLPCSYTLPTSGTLVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 30 - protein, 353 aa, Artificial Sequence
  Synthetic Polypeptide
MEPLPGWGSAPWSQAPTDNTFRLVPCVFLLNLLQRISAQPSCRQEEFLVGDECCPMCNPGYHVKQVCSEHTGTVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 31 - protein, 291 aa, Artificial Sequence
  Synthetic Polypeptide
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 32 - protein, 424 aa, Artificial Sequence
  Synthetic Polypeptide
MQLKCPCFVSLGTRQPVWKKLHVSSGFFSGLGLFLLLLSSLCAASAETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 33 - protein, 597 aa, Artificial Sequence
  Synthetic Polypeptide
MREDLLLGFLLLGLLWEAPVVSDYKDDDDKSGPGKELPVVWAQEGAPVHLPCSLKSPNLDPNFLRRGGVIWQHQPDSGQPTPIPALDLHQGMPSPRQPAPGRYTVLSVAPGGLRSGRQPLHPHVQLEERGLQRGDFSLWLRPALRTDAGEYHATVRLPNRALSCSLRLRVGQASMIASPSGVLKLSDWVLLNCSFSRPDRPVSVHWFQGQNRVPVYNSPRHFLAETFLLLPQVSPLDSGTWGCVLTYRDGFNVSITYNLKVLGLEPVAPLTVYAAEGSRVELPCHLPPGVGTPSLLIAKWTPPGGGPELPVAGKSGNFTLHLEAVGLAQAGTYTCSIHLQGQQLNATVTLAVITVTPKSFGLPGSRGKLLCEVTPASGKERFVWRPLNNLSRSCPGPVLEIQEARLLAERWQCQLYEGQRLLGATVYAAESSSGAHSARRILKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 34 - protein, 358 aa, Artificial Sequence
  Synthetic Polypeptide
MYVWVQQPTALLLLGLTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 35 - protein, 316 aa, Artificial Sequence
  Synthetic Polypeptide
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 36 - protein, 386 aa, Artificial Sequence
  Synthetic Polypeptide
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPALKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 37 - protein, 368 aa, Artificial Sequence
  Synthetic Polypeptide
MLLLLPILNLSLQLHPVAALFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 38 - protein, 479 aa, Artificial Sequence
  Synthetic Polypeptide
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQTLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 39 - protein, 378 aa, Artificial Sequence
  Synthetic Polypeptide
METDTLLLWVLLLWVPGSTGDLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLAAAHHHHHHV

SEQ ID NO: 40 - protein, 320 aa, Artificial Sequence
  Synthetic Polypeptide
METDTLLLWVLLLWVPGSTGDLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPLAAAHHHHHHV

SEQ ID NO: 41 - protein, 370 aa, Artificial Sequence
  Synthetic Polypeptide
METDTLLLWVLLLWVPGSTGDLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISRASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEAAAHHHHHHV

SEQ ID NO: 42 - protein, 298 aa, Artificial Sequence
  Synthetic Polypeptide
METDTLLLWVLLLWVPGSTGDLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLAAAHHHHHHV

SEQ ID NO: 43 - protein, 395 aa, Artificial Sequence
  Synthetic Polypeptide
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 44 - protein, 406 aa, Artificial Sequence
  Synthetic Polypeptide
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 45 - protein, 304 aa, Artificial Sequence
  Synthetic Polypeptide
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 46 - protein, 338 aa, Artificial Sequence
  Synthetic Polypeptide
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 47 - protein, 299 aa, Artificial Sequence
  Synthetic Polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 48 - protein, 340 aa, Artificial Sequence
  Synthetic Polypeptide
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 49 - protein, 27 aa, Artificial Sequence
  Synthetic Polypeptide
DGIFCSSSNFLGISFLLILMLILYSFI

SEQ ID NO: 50 - protein, 389 aa, Artificial Sequence
  Synthetic Polypeptide
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 51 - protein, 394 aa, Artificial Sequence
  Synthetic Polypeptide
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 52 - protein, 333 aa, Artificial Sequence
  Synthetic Polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 53 - protein, 307 aa, Artificial Sequence
  Synthetic Polypeptide
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 54 - protein, 349 aa, Artificial Sequence
  Synthetic Polypeptide
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 55 - protein, 349 aa, Artificial Sequence
  Synthetic Polypeptide
MEPPGDWGPPPWRSTPRTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 56 - protein, 403 aa, Artificial Sequence
  Synthetic Polypeptide
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 57 - protein, 597 aa, Artificial Sequence
  Synthetic Polypeptide
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRALKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 58 - protein, 361 aa, Artificial Sequence
  Synthetic Polypeptide
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 59 - protein, 317 aa, Artificial Sequence
  Synthetic Polypeptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 60 - protein, 385 aa, Artificial Sequence
  Synthetic Polypeptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 61 - protein, 367 aa, Artificial Sequence
  Synthetic Polypeptide
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 62 - protein, 288 aa, Artificial Sequence
  Synthetic Polypeptide
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVALKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 63 - protein, 312 aa, Artificial Sequence
  Synthetic Polypeptide
MNSGLQLVFFVLTLKGIQGEGSHHHHHHVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRDIGGGGSGGGGSDYKDDDDKDGIFCSSSNFLGISFLLILMLILYSFI

SEQ ID NO: 64 - protein, 320 aa, Artificial Sequence
  Synthetic Polypeptide
MERHWIFLLLLSVTAGVHSQVGSHHHHHHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRLKEFGGGGSGGGGSDYKDDDDKDGIFCSSSNFLGISFLLILMLILYSFI

SEQ ID NO: 65 - protein, 382 aa, Artificial Sequence
  Synthetic Polypeptide
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 66 - protein, 330 aa, Artificial Sequence
  Synthetic Polypeptide
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRKDPGHLVEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAARGRRKR

SEQ ID NO: 67 - protein, 296 aa, Artificial Sequence
  Synthetic Polypeptide
MGLTLLLLLLLGLEGQGIVGSLPEVLQAPVGSSILVQCHYRLQDVKAQKVWCRFLPEGCQPLVSSAVDRRAPAGRRTFLTDLGGGLLQVEMVTLQEEDAGEYGCMVDGARGPQILHRVSLNILPPEEEEETHKIGSLAENLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 68 - protein, 785 aa, Artificial Sequence
  Synthetic Polypeptide
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEILDVSGNGWTVDITGNFSNAISKSQAFSLILAHHIMGAGFGFHNIKDPDQNTFAGLARSSVRHLDLSHGFVFSLNSRVFETLKDLKVLNLAYNKINKIADEAFYGLDNLQVLNLSYNLLGELYSSNFYGLPKVAYIDLQKNHIAIIQDQTFKFLEKLRTLDLRDNALTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSFSGVSLFSLSTEGCDEEEVLKGSMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAARV

SEQ ID NO: 69 - protein, 756 aa, Artificial Sequence
  Synthetic Polypeptide
MLKMLSFKLLLLAVALGFFEGDAKFGERNEGSGARRRRCLNGNPPKRLKRRDRRMMSQLELLSGGEMLCGGFYPRLSCCLRSDSPGLGRLENKIFSVTNNTECGKLLEEIKCALCSPHSQSLFHSPEREVLERDLVLPLLCKDYCKEFFYTCRGHIPGFLQTTADEFCFYYARKDGGLCFPDFPRKQVRGPASNYLDQMEEYDKVEEISRKHKHNCFCIQEVVSGLRQPVGALHSGDGSQRLFILEKEGYVKILTPEGEIFKEPYLDIHKLVQSGIKGGDERGLLSLAFHPNYKKNGKLYVSYTTNQERWAIGPHDHILRVVEYTVSRKNPHQVDLRTARVFLEVAELHRKHLGGQLLFGPDGFLYIILGDGMITLDDMEEMDGLSDFTGSVLRLDVDTDMCNVPYSIPRSNPHFNSTNQPPEVFAHGLHDPGRCAVDRHPTDININLTILCSDSNGKNRSSARILQIIKGKDYESEPSLLEFKPFSNGPLVGGFVYRGCQSERLYGSYVFGDRNGNFLTLQQSPVTKQWQEKPLCLGTSGSCRGYFSGHILGFGEDELGEVYILSSSKSMTQTHNGKLYKIVDPKRPLMPEECRATVQPLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 70 - protein, 905 aa, Artificial Sequence
  Synthetic Polypeptide
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 71 - protein, 911 aa, Artificial Sequence
  Synthetic Polypeptide
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGITRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIELKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 72 - protein, 523 aa, Artificial Sequence
  Synthetic Polypeptide
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATGSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 73 - protein, 470 aa, Artificial Sequence
  Synthetic Polypeptide
MGIGRSEGGRRGAALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFKLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 74 - protein, 792 aa, Artificial Sequence
  Synthetic Polypeptide
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 75 - protein, 321 aa, Artificial Sequence
  Synthetic Polypeptide
MEPLRLLILLFVTELSGAHNTTVFQGVAGQSLQVSCPYDSMKHWGRRKAWCRQLGEKGPCQRVVSTHNLWLLSFLRRWNGSTAIKDDTLGGTLTITLRNLQPHDAGLYQCQSLHGSEADTLRKVLVEVLADPLDHRDAGDLWFPGESESFEDAHVEHSISRSLLELKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 76 - protein, 419 aa, Artificial Sequence
  Synthetic Polypeptide
MAPAFLLLLLLWPQGCVSGPSADSVYTKVRLLEGETLSVQCSYKGYKNRVEGKVWCKIRKKKCEPGFARVWVKGPRYLLQDDAQAKVVNITMVALKLQDSGRYWCMRNTSGILYPLMGFQLDVSPAPQSERNIPFTHLDNILKSGTVTTGQAPTSGPDAPFTTGVMVFTPGLITLPRLLASTRPASKTGYSFTATSTTSQGPRRTMGSQTVTASPSNARDSSAGPESISTKSGDLSTRSPTTGLCLTSRSLLNRLPSMPLKEFIEGRMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 77 - protein, 680 aa, Artificial Sequence
  Synthetic Polypeptide
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPRLCRHGGTCHNEVGSYRCVCRATHTGPNCERPYVPCSPSPCQNGGTCRPTGDVTHECACLPGFTGQNCEENIDDCPGNNCKNGGACVDGVNTYNCPCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCMCMPGYEGVHCEVNTDECASSPCLHNGRCLDKINEFQCECPTGFTGHLCQEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 78 - protein, 664 aa, Artificial Sequence
  Synthetic Polypeptide
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIHQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANVTTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 79 - protein, 406 aa, Artificial Sequence
  Synthetic Polypeptide
METDTLLLWVLLLWVPGSTGDLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGAAAHHHHHHV

SEQ ID NO: 80 - protein, 535 aa, Artificial Sequence
  Synthetic Polypeptide
MARRSAFPAAALWLWSILLCLLALRAEAGPPQEESLYLWIDAHQARVLIGFEEDILIVSEGKMAPFTHDFRKAQQRMPAIPVNIHSMNFTWQAAGQAEYFYEFLSLRSLDKGIMADPTVNVPLLGTVPHKASVVQVGFPCLGKQDGVAAFEVDVIVMNSEGNTILKTPQNAIFFKTCQQAECPGGCRNGGFCNERRICECPDGFHGPHCEKALCTPRCMNGGLCVTPGFCICPPGFYGVNCDKANCSTTCFNGGTCFYPGKCICPPGLEGEQCEISKCPQPCRNGGKCIGKSKCKCSKGYQGDLCSKPVCEPGCGAHGTCHEPNKCQCQEGWHGRHCNKRYEASLIHALRPAGAQLRQHTPSLKKAEERRDPPESNYIWLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 81 - protein, 422 aa, Artificial Sequence
  Synthetic Polypeptide
MMALGAAGATRVFVAMVAAALGGHPLLGVSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRHLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 82 - protein, 451 aa, Artificial Sequence
  Synthetic Polypeptide
MLQGPGSLLLLFLASHCCLGSARGLFLFGQPDFSYKRSNCKPIPVNLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPATEEAPKVCEACKNKNDDDNDIMETLCKNDFALKIKVKEITYINRDTKIILETKSKTIYKLNGVSERDLKKSVLWLKDSLQCTCEEMNDINAPYLVMGQKQGGELVITSVKRWQKGQREFKRISRSIRKLQCLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 83 - protein, 620 aa, Artificial Sequence
  Synthetic Polypeptide
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPPVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 84 - protein, 667 aa, Artificial Sequence
  Synthetic Polypeptide
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCELKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 85 - protein, 606 aa, Artificial Sequence
  Synthetic Polypeptide
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDINLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 86 - protein, 675 aa, Artificial Sequence
  Synthetic Polypeptide
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 87 - protein, 417 aa, Artificial Sequence
  Synthetic Polypeptide
MVSYWDTGVLLCALLSCLLLTGSSSGSKIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQGGGGSGGGGSLKIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHGGGGSGGGGSLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 88 - protein, 273 aa, Artificial Sequence
  Synthetic Polypeptide
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 89 - protein, 313 aa, Artificial Sequence
  Synthetic Polypeptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 90 - protein, 934 aa, Artificial Sequence
  Synthetic Polypeptide
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPILKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 91 - protein, 468 aa, Artificial Sequence
  Synthetic Polypeptide
MAKVFSFILVTTALTMGREISALEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVILKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 92 - protein, 463 aa, Artificial Sequence
  Synthetic Polypeptide
MTELPAPLSYFQNAQMSEDNHLSNTNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGAFIFYSVLVGKASATASGDWNTTIACFVAILIGLCLTLLLLAIFKKALPALPISITFGLVFYFATDYLVQPFMDQLAFHQFYI

SEQ ID NO: 93 - protein, 361 aa, Artificial Sequence
  Synthetic Polypeptide
MMAPFASLASGILLLLSLIASSKACSCAPPHPQTAFCNSDLVIRAKFMGSPEINETTLYQRYKIKMTKMLKGFKAVGNAADIRYAYTPVMESLCGYAHKSQNRSEEFLITGRLRNGNLHISACSFLVPWRTLSPAQQRAFSKTYSAGCGVCTVFPCLSIPCKLESDTHCLWTDQVLVGSEDYQSRHFACLPRNPGLCTWRSLGARLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 94 - protein, 376 aa, Artificial Sequence
  Synthetic Polypeptide
MGAAARSLRLALGLLLLATLLRPADACSCSPVHPQQAFCNADVVIRAKAVSEKEVDSGNDIYGNPIKRIQYEIKQIKMFKGPDKDIEFIYTAPSSAVCGVSLDVGGKKEYLIAGKAEGDGKMHITLCDFIVPWDTLSITQKKSLNHRYQMGCECKITRCPMIPCYISSPDECLWMDWVTEKSINGHQAKFFACIKRSDGSCAWYRGAAPPKQEFLDIEDPLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 95 - protein, 367 aa, Artificial Sequence
  Synthetic Polypeptide
MTPWLGLVVLLSCWSLGHWGAEACTCSPSHSQDAFCNSDIVIRAKVVGKKLVTEGPFGTLVYTIKQMKMYRGFSKMPHVQYIHTEASESLCGLKLEVNKYQYLLTGRVYEGKMYTGLCNFVERWDHLTLSQRKGLNYRYHLGCNCKINSCYYLPCFVTSKNECLWTDMLSNFGYPGYQSKHYACIRQKGGYCSWYRGWAPPDKSISNATDPLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 96 - protein, 380 aa, Artificial Sequence
  Synthetic Polypeptide
MPWSPLAALSWALVLRLLALLWPPGRGEACSCAPAHPQQHFCHSALVIRAKISSEKVVPASKDPADTQKLIRYEIKQIKMFKGFEKAKDIQYVYTPFDSSLCGVKLETNSHKQYLLTGQILSDGKVFIHLCNYIEPWEDLSLVQRESLNHHYHQNCGCQITTCYAVPCTISAPNECLWTDWLLERKLYGYQAQHYVCMKHVDGICSWYRGHLHLRKEYVDIIQPLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 97 - protein, 252 aa, Artificial Sequence
  Synthetic Polypeptide
MMAPFASLASGILLLLSLIASSKACSCAPPHPQTAFCNSDLVIRAKFMGSPEINETTLYQRYKIKMTKMLKGFKAVGNAADIRYAYTPVMESLCGYAHKSQNRSEEFLITGRLRNGNLHISACSFLVPWRTLSPAQQRAFSKTYSAGCGVCTVFPCLSIPCKLESDTHCLWTDQVLVGSEDYQSRHFACLPRNPGLCTWRSLGARTRTRPLEQKLISEEDLAANDDGIFCSSSNFLGISFLLILMLILYSFI

SEQ ID NO: 98 - protein, 267 aa, Artificial Sequence
  Synthetic Polypeptide
MGAAARSLRLALGLLLLATLLRPADACSCSPVHPQQAFCNADVVIRAKAVSEKEVDSGNDIYGNPIKRIQYEIKQIKMFKGPDKDIEFIYTAPSSAVCGVSLDVGGKKEYLIAGKAEGDGKMHITLCDFIVPWDTLSITQKKSLNHRYQMGCECKITRCPMIPCYISSPDECLWMDWVTEKSINGHQAKFFACIKRSDGSCAWYRGAAPPKQEFLDIEDPTRTRPLEQKLISEEDLAANDDGIFCSSSNFLGISFLLILMLILYSFI

SEQ ID NO: 99 - protein, 258 aa, Artificial Sequence
  Synthetic Polypeptide
MTPWLGLVVLLSCWSLGHWGAEACTCSPSHSQDAFCNSDIVIRAKVVGKKLVTEGPFGTLVYTIKQMKMYRGFSKMPHVQYIHTEASESLCGLKLEVNKYQYLLTGRVYEGKMYTGLCNFVERWDHLTLSQRKGLNYRYHLGCNCKINSCYYLPCFVTSKNECLWTDMLSNFGYPGYQSKHYACIRQKGGYCSWYRGWAPPDKSISNATDPTRTRPLEQKLISEEDLAANDDGIFCSSSNFLGISFLLILMLILYSFI

SEQ ID NO: 100 - protein, 271 aa, Artificial Sequence
  Synthetic Polypeptide
MPWSPLAALSWALVLRLLALLWPPGRGEACSCAPAHPQQHFCHSALVIRAKISSEKVVPASKDPADTQKLIRYEIKQIKMFKGFEKAKDIQYVYTPFDSSLCGVKLETNSHKQYLLTGQILSDGKVFIHLCNYIEPWEDLSLVQRESLNHHYHQNCGCQITTCYAVPCTISAPNECLWTDWLLERKLYGYQAQHYVCMKHVDGICSWYRGHLHLRKEYVDIIQPTRTRPLEQKLISEEDLAANDDGIFCSSSNFLGISFLLILMLILYSFI

SEQ ID NO: 101 - protein, 616 aa, Artificial Sequence
  Synthetic Polypeptide
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVLKEFGGGGSGGGGSGGGGSMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKGGGGSGGGGSGGGGSLQISTRISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGTRAAAHHHHHHV

SEQ ID NO: 102 - protein, 753 aa, Artificial Sequence
  Synthetic Polypeptide
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATGSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISGGGGSGGGGSIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQGGGGSGGGGSLKIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHAAAHHHHHHV

SEQ ID NO: 103 - protein, 4 aa, Artificial Sequence
  Synthetic Polypeptide
GGGS

SEQ ID NO: 104 - protein, 8 aa, Artificial Sequence
  Synthetic Polypeptide
GGGSGGGS

SEQ ID NO: 105 - protein, 563 aa, Artificial Sequence
  Synthetic Polypeptide
WTMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANLKEFGGGGSGGGGSGGGGSMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKGGGGSGGGGSGGGGSLQISTRAAAHHHHHHV

SEQ ID NO: 106 - protein, 561 aa, Artificial Sequence
  Synthetic Polypeptide
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTIFLDRHPIAFMLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANLKEFGGGGSGGGGSGGGGSMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKGGGGSGGGGSGGGGSLQISTRAAAHHHHHHV

SEQ ID NO: 107 - protein, 692 aa, Artificial Sequence
  Synthetic Polypeptide
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

SEQ ID NO: 108 - protein, 945 aa, Artificial Sequence
  Synthetic Polypeptide
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQLKEFMGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISGGGGSGGGGSQVTLKESGPGILQPSQTLSLTCSFSGFSLNTFGMGVSWIRQPSGKGLEWLAHIFWDDDKHYNPSLKSRLTISKDTSNNQVFLKITTVDTADTATYYCVRYGFDGFPYWGQGTLVTVSAGGGGSGGGGSGGGGSDVVMTQTPLSLPVSLGDQASISCRSNQSLVHSNGNTYLHWYLQKPGQSPKLLIYKVSNRFSGVPDRFSGSGSGTDFTLKISRVEAEDLGFYFCSQSTRVPWTFGGGTKLELKRAAAHHHHHHV

SEQ ID NO: 109 - protein, 152 aa, Artificial Sequence
  Synthetic Polypeptide
MGYQGPPGPPGPPGIPGNHGNNGNNGATGHEGAKGEKGDKGDLGPRGERGQHGPKGEKGYPGIPGYCGPPGPPGPPGIPGNHGNNGNNGATGHEGAKGEKGDKGDLGPRGERGQHGPKGEKGYPGIPDYKDDDDKLQISSTVAAAHHHHHHV

(19) United States
(12) Patent Application Publication
(10) Pub. No.: US 2022/0324943 A1
(43) Pub. Date: Oct. 13, 2022

(54) MULTIMERIC PROTEIN DOMAINS FOR MULTIFUNCTIONALITY AND ENHANCED SECRETION OF THERAPEUTIC PROTEINS

(71) Applicant: University of Florida Research Foundation, Incorporated, Gainesville, FL (US)

(72) Inventors: Todd Eliot Golde, Gainesville, FL (US); Yong Ran, Gainesville, FL (US); Marshall S. Goodwin, Gainesville, FL (US); Pedro Cruz, Gainesville, FL (US); Yona Levites, Gainesville, FL (US)

(73) Assignee: University of Florida Research Foundation, Incorporated, Gainesville, FL (US)

(21) Appl. No.: 17/629,356

(22) PCT Filed: Jul. 22, 2020
(86) PCT No.: PCT/US2020/043011

§ 371 Date: Jan. 21, 2022

Related U.S. Application Data
(60) Provisional application No. 62/876,850, filed on Jul. 22, 2019.

(51) Int. Cl. C07K 14/705; C07K 14/47; C07K 14/525; C07K 14/71

(52) U.S. Cl. CPC C07K 14/70596; C07K 14/4711; C07K 14/525; C07K 14/71; C07K 14/4747; C07K 14/70521; C07K 14/70532; C07K 2319/21; C07K 2319/43; C07K 2317/622

(57) ABSTRACT

Provided herein are compositions and methods for the stable production of bioactive small peptides of interest through delivery to target cells based on the fusion of small peptides of interest to a collagen domain of a C1qTNF protein to produce a novel scaffold protein capable of multimerization. Advantageously, the fusion proteins, compositions and methods of the present disclosure meet existing needs in the art by providing for higher stable expression and longer stability of intracellular and secretable peptides of interest. Additionally, the fusion proteins, compositions and methods of the present disclosure provide for improved binding affinity of expressed receptor peptides with ligand binding partners in the target cell. Further provided herein are polynucleotide constructs encoding the described fusion proteins and recombinant adeno-associated viral particles comprising these polynucleotides. Also provided herein are pharmaceutical compositions and nanoparticles that comprise the described fusion proteins. Further provided herein are methods of treating a subject by administering the described fusion proteins, rAAV particles, compositions and/or nanoparticles.

### RELATED APPLICATIONS

This application claims the benefit under 35 U.S.C 119(e) of the filing date of U.S. Provisional Application Ser. No. 62/876,850, filed Jul. 22, 2019, the entire contents of which are incorporated herein by reference.

### FEDERALLY SPONSORED RESEARCH

This invention was made with government support under P01 CA166009 awarded by the National Institutes of Health. The government has certain rights in the invention.

### BACKGROUND OF THE INVENTION

Bioactive small peptides, either occurring naturally or produced synthetically, have gained increasing interest as research tools and as pharmacological drugs in recent years. In particular, these bioactive small peptides play key roles in various physiological, as well as pathological processes, and thus are promising targets for therapeutic interventions for many pathological conditions. Further, bioactive small peptides have gained use as agonists or antagonists of various biological processes with enhanced target binding affinity and specificity as compared to conventional agents. Despite these advances, the use of small peptides as exogenous gene delivery candidates has been hampered by limited expression efficiency, low binding affinity, and short half-life in cells (due primarily to the small peptides susceptibility to cellular proteases for degradation).

One strategy that has been developed to overcome these problems of exogenous administration of small peptides is to generate the peptide intracellularly by using high-expression vectors administered to the cell or tissue of interest. Investigators have employed different expression systems to express the small peptide as part of a fusion peptide with scaffold proteins or other reporter proteins, but this strategy has been unsuccessful for small peptides that are exclusively active in, or are more highly active in, their free peptide forms.

Separately, effective therapies for Alzheimer's Disease (AD), Parkinson's Disease (PD) and related neurodegenerative diseases remains a significant unmet medical need. Immunotherapeutic approaches remain a major focus of the effort to develop effective disease modifying therapeutics for AD. As opposed to inhibitors that target amyloid-beta (Aβ, or Abeta) plaque production in the brain, anti-Aβ immunotherapies have potential to clear preexisting deposits, neutralize toxic Aβ aggregates, or both; thus, there is better rationale for testing immunotherapies in patients with preexisting pathology.

### SUMMARY OF THE INVENTION

The present disclosure provides novel compositions and methods for stable production of bioactive small peptides of interest through delivery to target cells. Aspects of the present disclosure provide for exogenous expression of peptides of interest as the product of a larger scaffold protein comprising a collagen domain of a C1QTNF protein.

The fusion proteins, compositions and methods of the present disclosure meet existing needs in the art by providing for higher stable expression and longer stability of the intracellular and secretable peptides of interest. Additionally, the fusion proteins, compositions and methods of the present disclosure provide for improved binding affinity of expressed receptor peptides with ligand binding partners in the target cell, including binding of immunotherapeutic peptides with amyloid-beta ligands.

The present disclosure is based, at least in part, on the understanding that collagen is an extracellular matrix protein that is the main structural protein of various connective tissues in mammals. It is that contains one or more triple-helical regions (collagenous domains) with a repeating triplet sequence Glycine-X-Y, where X and Y are frequently proline (amino acid code, P or Pro) or hydroxyproline (amino acid code, O or Hyp). See Lodish et al., MOLECULAR CELL BIOLOGY, 4^{th} edition, Section 22.3, New York: W. H. Freeman, ed.; 2000. The presence of such triplets allows three collagen polypeptide chains (α-chains) to fold into a triple-helical conformation within the cell.

Many collagen-like proteins with collagenous domains are present in human serum and serve as an innate immune system in protection from infectious organisms. These include complement protein C1q. A common structural feature among these “defense collagen” molecules is their association into multi-trimeric protein units with a target-binding domain at the C-terminus. Consequently, multimerization may increase the functional affinity of the binding domain of these defense collagen molecules. Trimerization of heterologous fusion proteins containing collagenous domains has been accomplished by fusing a homogeneous or heterologous trimerization domain to a collagenous domain to drive the collagen triplex formation. See US Publication No. 2008/0176247, herein incorporated by reference.

A high-expression vector system under active investigation for its clinical potential is the recombinant adeno-associated viral (rAAV) vector. Limited expression levels and poor half-life of secretable small peptides remain an obstacle in the development of many rAAV applications. Some groups have introduced secretion signal and cell-penetrating peptide sequences in conjugation, or a fusion protein, with the small peptide of interest. See, e.g., US Patent Publication No. 2012/0157513, incorporated herein by reference.

The peptide delivery strategy of the present disclosure combines rAAV systems with a fusion protein comprising a genetically optimized collagen domain (CD) of a human C1qTNF protein. This domain is referred to herein as the “C1qTNF collagen domain,” or “CCD.” In various aspects, the collagen domains of the fusion proteins provided herein are derived from a C1qTNF protein. In particular embodiments, the collagen domain is derived from a C1qTNF3 protein. In some embodiments, the collagen domain is derived from a C1qTNF5 protein. In various embodiments, the collagen domain is derived from a mammalian C1qTNF protein, such as a human C1qTNF protein.

Peptide expression and delivery using the CCD fusion proteins of the present disclosure surprisingly provides solutions to each of the following long-felt needs: 1) achievement of greater than ten-fold expression levels of small peptides of interest in vitro and in vivo, 2) promotion of multimerization of the peptide of interest, thus enhancing ligand binding affinity by exploiting avidity, and 3) stabilization of half-life of the peptide products.

Accordingly, in some aspects, the present disclosure provides fusion proteins comprising (i) a C1qTNF protein collagen domain (CCD) and (ii) a first heterologous peptide. In particular embodiments, the CCD is derived from a C1qTNF of human origin. In some embodiments, the CCD is a C1qTNF3 CD, and may be derived from a human C1qTNF3 CD.

The heterologous peptide may be positioned at the N-terminus or the C-terminus of the CCD fusion protein. Thus, in some embodiments, disclosed fusion proteins comprise the structure NH₂-[first heterologous peptide]-[CCD]-COOH or NH₂-[CCD][first heterologous peptide]-COOH, wherein each instance of “H” indicates the presence of an optional linker sequence.

In some embodiments, the disclosed fusion proteins further comprise a second heterologous peptide. Accordingly, in such embodiments, the fusion proteins may comprise the structure NH₂-[first heterologous peptide]-[CCD]-[second heterologous peptide]-COOH, NH₂-[CCD]-[first heterologous peptide]-[second heterologous peptide]-COOH, or NH₂-[first heterologous peptide]-[second heterologous peptide]-[CCD]-COOH, wherein each instance of “]-[” indicates the presence of an optional linker sequence. Such fusion proteins may have bispecificity for two or more ligands.

In certain aspects, the one or more heterologous peptides of the fusion proteins may comprise a single-chain variable fragment (scFv) or an immune checkpoint modulator.

In certain aspects, the present disclosure provides multimeric fusion proteins comprising two or more monomers comprising a fusion protein as described above. The monomers may be identical or different. In some embodiments, the multimeric fusion protein comprises a trimer or a hexamer.

In certain aspects, the present disclosure provides polynucleotides encoding one or more of the fusion proteins or multimeric fusion proteins. Further provided are expression vectors (or constructs) comprising such polynucleotides. Also provided are recombinant AAV particles comprising such polynucleotides or expression vectors.

In other aspects, the disclosure provides pharmaceutical compositions that comprise a fusion protein or multimeric protein as described above, or an expression vector as described above, and a pharmaceutically acceptable excipient. Also provided are nanoparticles comprising the fusion proteins.

In other aspects, the disclosure provides methods of treatment of a subject having a disease, disorder or condition comprising administering to the subject one or more CCD fusion proteins as described above. Also provided are methods and kits for detecting the presence of one or more analytes in a cell, tissue or organ comprising administering one or more CCD fusion proteins wherein the protein has binding affinity for the one or more analytes.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1A is a schematic illustrating the probable trimeric structure of a CCD fusion protein comprising two heterologous peptides (“Fusion protein 1” and “Fusion protein 2”). A monomeric fusion protein is also illustrated for reference. Disulfide bridges between cysteine residues are denoted with a “Cys”. In FIG. 1B, the amino acid of the optimized CCD sequence from C1qTNF3, fused to a FLAG tag and poly(A) tail—which corresponds to SEQ ID NO: 11—is shown.

FIG. 2 depicts a Western blot image that show that fusion with CCD domains substantially increases the expression and secretion levels of various heterologous peptide partners following administration of an rAAV particle comprising a plasmid encoding various CCD-heterologous peptide fusion proteins by PEI transfection into HEK293 cells. Heterologous peptide partners consisted of soluble Lag3, soluble PD-1, CTLA4, NOTCH1, DLL3 and Aβ9 single-chain variable fragment (scFv). The top panel shows a Western blot of cell lysate in the absence of a reducing agent. The bottom panel shows a Western blot of conditioned media following cell harvest.

FIG. 3 depicts Western blot images that indicate that the CCD fusion proteins form multimeric structures. The left panel shows an SDS-PAGE analysis of several elution fractions of media following lysis of HEK293 cells transfected with rAAV nucleic acid vector encoding sLag3-CCD_FLAG. Each series of bands represents a different multimer (e.g. hexamer, trimer, dimer, monomer). The right panel shows an SDS-PAGE analysis of five of these media elution fractions after treatment with different temperature and reducing conditions.

FIG. 4 shows the stability of sB7H3-CCD fusion proteins following storage at room temperature and 4° C. for 12 days and 30 days, respectively.

FIG. 5 shows the enhanced expression of a CCD fusion protein comprising single chain antibody fragment against amyloid beta peptide 5 (Aβ5 scFv CCD) after administration of an rAAV particle comprising a polynucleotide encoding this fusion protein to a mouse model.

FIGS. 6A-6E are examples of bispecific CCD fusion proteins comprising functional multivalent tau and Aβ binding single chain antibody fragment (scFv, or “nanobody”) heterologous peptides. FIG. 6A is a schematic of an exemplary bispecific CCD nanobody containing 94-3A6 scFv targeting tau and scFv targeting Aβ9 (the CCD is represented as “Col D”). FIG. 6B shows the binding to both tau and Aβ by ELISA of the bispecific nanobody CCD fusion protein (the CCD is represented as “collagen”). FIG. 6C shows the expression levels of the fusion protein in cell media. FIG. 6D shows staining of amyloid plaque pathology after administration of a rAAV2/8 comprising a polynucleotide encoding a bispecific CCD fusion protein comprising scFv's targeting tau and Aβ in CRND8 mice and cells derived from human Alzheimer's Disease patient (“Human AD”). FIG. 6E is a surface plasmon resonance sensorgram depicting biolayer interferometry results showing the ability of the fusion protein to sequentially bind tau and Aβ.

FIG. 7 shows that rAAV administration of the bispecific CCD fusion proteins comprising anti-Aβ9 scFv and B11 scFv targeting VEGFR-2 reduces amyloid plaque depositions in vivo in CRND8 transgenic mice that overexpress amyloid precursor protein (APP). “collagen”=a CCD domain.

FIGS. 8A-8C show that CCD fusion proteins comprising a VEGFR1 decoy receptor successfully inhibit growth of cells in in vitro and in vivo assays in a human HUVEC cell line. FIG. 8A is a schematic showing the HUVEC tube formation assay. The enzymatic MTT reduction reaction that is performed in an MTT assay is also shown. Further shown in a chart of various molecular mediators of angiogenesis, several of which have been selected for CCD fusion protein generation and evaluation, e.g. in HUVEC tube formation and MTT assays. The immunostaining images of FIG. 8B illustrates the results of a cell tube formation assay showing inhibition of HUVEC tube formation by isolated and purified human VEGFR1-CCD fusion protein and a fusion protein comprising human VEGFR1 domain 2 and human VEGFR2 domain 3 and a CCD domain (“VLV2-CCD”) and conditioned media. FIG. 8C shows MTT activity. “Collagen” and “Collagen protein” indicate administration of the CCD domain alone.

FIG. 9 is a schematic that shows various transmembrane and extracellular immune synapse heterologous peptides expressed on antigen presenting cells and T cells that have been selected for cloning and fusion to CCD domains of the disclosure.

FIG. 10 is a schematic that shows the transduction pathways of various cell signaling (anti-proliferative) heterologous peptides that were selected for cloning and fusion.

FIG. 11 shows overexpression and secretion levels of various CCD fusion decoy proteins comprising human-derived (black) or mouse-derived (red) immune checkpoint proteins after rAAV-mediated expression in cells, lysis and isolation of media/supernatant from cell lysate. CCD fusions with mouse-derived peptides were administered to mouse tissues, and fusions with human-derived peptides were administered into human tissues. For many of the immune synapse proteins shown, ectodomain fusion to the CCD domain generates expression and secretion at high levels in both mouse and human constructs. These Western blots show successful generation of over 50 CCD fusion proteins. The top panel shows a reduced Western blot of cell lysate. The bottom panel shows a Western blot of media in the absence of reducing agent. Unconjugated CCD domain (“Collagen”) and rAAV vector alone were used as negative controls.

FIG. 12 shows Western blots indicating the overexpression and secretion of various CCD fusion proteins comprising cell signaling heterologous peptides in both human and mouse models. Fusion of the signaling peptide to a CCD domain generates expression and secretion at high levels in both mouse and human. Conditioned media and lysate in the presence and absence of reducing agent were subjected to SDS-PAGE analysis. Unconjugated CCD domain (“Collagen”) was used as a control.

FIG. 13 shows Western blots indicating the overexpression and secretion of various CCD fusion proteins in both human and mouse tissues. The blot on the far right shows an evaluation of overexpression and secretion differences when the CCD is positioned at the N-terminus or C-terminus of a soluble Toll-like Receptor 5 (sTLR5) peptide (“Col” and “CD” represent the CCD), where cells were harvested 48 hours post-transfection.

FIG. 14 shows Western blots indicating expression levels of Icos-CCD fusion proteins after rAAV-mediated administration to HEK 293T cells, in the presence or absence of media containing Icos ligands (“IcosL”). “Collagen” represents the CCD.

FIGS. 15A-15C show Western blots indicating that several of the sLag3, NOTCH1, HHIP, VEGFR1, Tgfbr2, PD-1, HAVCR2, CTLA4, BTLA, SFRP1 and EGFR CCD fusion proteins bind to the cell surface after incubation of purified fusion protein with human and mouse cells, followed by lysis and separation. The SDS-PAGE analyses in FIGS. 15A and 15B were performed using HEK 293T cells, while that of FIG. 15C was performed using mouse glioma 261 (GL261) cells. “ms” designates mouse sequences, and “h” or “hs” designates human sequences. FIG. 15D shows an MTT assay in GL261 cells for evaluation of six CCD fusion proteins for any decoy receptor activity. Media and unconjugated CCD alone were used as controls.

FIG. 16 shows Western blots indicating expression and secretion levels of rAAV6-mediated delivery of various CCD fusion proteins with human heterologous peptides to MB-MDA-231 epithelial human breast cancer cells. Cells were harvested later than in previous experiments—at 72 hours post-transfection.

FIG. 17 show Western blots indicating expression and secretion levels of various CCD fusion protein decoy receptors in HEK293 cells. The blot on the far right indicates the effect of multiple media changes on EGFR-CDD expression levels.

FIG. 18 shows SDS-PAGE Coomassie staining indicating expression and secretion levels of various CCD fusion protein decoy receptors in HEK293 cells after single step His₆-tag purification was performed.

FIG. 19A is a schematic of three exemplary fusion proteins according to the disclosure. The heterologous peptides are positioned N-terminal to the CCD. FIG. 19B is a reference figure that illustrates the expected results of an MTT assay as performed according to published specifications.

FIG. 20 illustrates the results of an MTT assay (top panels) and tube formation assays (bottom panels) performed after a 36 hour incubation period of HUVEC cells cultured in complete media to 90% confluence.

FIG. 21A depicts an SPR sensorgram from a binding analysis in which a solution of FGFR1-CCD was injected over immobilized acidic FGF and basic FGF ligands. This sensorgram consists of two phases: (Left) In the association phase, as the interaction between FGFR1-CCD and FGF ligands increases, the SPR signal increases [displayed in response units (RU)]. Maximal binding is indicated at the “AlignX” time. (Right) After 600 seconds, injection of FGFR1-CCD into the biosensor was stopped and the system was switched back to buffer in the dissociation phase. VEGFA, a null ligand, was used as a negative control. FIG. 21B illustrates the results of an MTT assay of HUVEC cells into which rAAV encoding FGFR1-CCD was administered. GFP and unconjugated CCD were used as controls.

FIG. 22 shows Western blots indicating expression and secretion levels of CCD domains derived from two C1QTNF proteins—C1QTNF3 (col3) and C1QTNF5 (col5)—in an assessment of optimal C1QTNF type for use in multimeric fusion proteins.

FIG. 23 shows results of activity assays for multimeric fusions containing CCD and glucocerebrosidase and CCD and Aβ9 scFv.

FIG. 24 shows results of a MTT assay of gancyclovir (GCV) on HSV-TK-SR39 transfected MC38 cell.

FIG. 25 shows a plot of serums levels of circulating single-chain variable fragment (scFv) following administration of α-Aβ5 scFv-CCD multimeric fusion protein constructs to mice. This assay was designed to assess the half-life of these fusion proteins.

### DEFINITIONS

As used in the specification and claims, the singular term “a”, “an” and “the” include plural references unless the context clearly dictates otherwise. For example, the term “a cell” includes a plurality of cells, including mixtures thereof.

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein).

The term “administration” or “administering” includes routes of introducing the compound of the invention(s) to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally affect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a pro-drug form which is converted into its active metabolite, or more active metabolite in vivo.

The term “agent” refers to a small molecule compound, a polypeptide, polynucleotide, or fragment, or analog thereof, or other biologically active molecule.

As used herein, an “amyloid-related disease or disorder” includes Alzheimer's Disease (AD) and Parkinson's Disease (PD). Exemplary diseases, disorders or conditions of the disclosure include, but are not limited to, AD, Parkinson's Disease, Amyotrophic Lateral Sclerosis (“ALS”), Multiple Sclerosis (“MS”), Stroke and Frontal temporal Dementia.

As used herein, “heterologous peptide” refers to a biologically active peptide. In certain embodiments, the heterologous peptide is a small bioactive peptide, e.g. one that is encoded by a cDNA sequence that may be packaged into a single rAAV nucleic acid segment that further comprises a CCD domain-encoding nucleotide sequence, e.g. a cDNA sequence having a length less than about 4 kilobases (kB). In various embodiments, the heterologous peptides of the disclosure are encoded by cDNA sequences of less than 3 kB, less than 2 kB, and/or less than 1 kB. Heterologous peptides may be intracellular or extracellular (secretable). Exemplary heterologous peptides include, but are not limited to, single-chain antibody fragments, ectodomains of transmembrane receptors, decoy receptors, Notch receptors and ligands, and immune checkpoint modulators. A heterologous peptide may contain a “binding domain,” which includes, but is not limited to, an antibody binding domain or a fragment thereof (e.g., a single-chain antigen-binding fragment).

An “ectodomain” refers to the domain of a transmembrane protein that extends into the extracellular space.

An “antibody” refers to an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The term “antigen-binding fragment” of an antibody (or simply “antibody fragment”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Multispecific and bispecific antibody constructs are well known in the art and described and characterized in Kontermann (ed.), Bispecific Antibodies, Springer, NY (2011), and Spiess et al., Mol. Immunol. 67(2):96-106 (2015), each of which are incorporated by reference herein.

Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

Variable regions can be linked to encode single chain Fv regions. Multiple Fv regions can be linked to confer binding ability to more than one target or chimeric heavy and light chain combinations can be employed. Any appropriate method may be used for cloning of antibody variable regions and generation of recombinant antibodies.

The term “bispecific,” as used herein, signifies having two different antigen binding domains, each domain being directed against a different ligand or epitope.

As used herein, the term “collagen scaffold domain” is a collagenous or collagen-like domain which allows for formation of a triplex structure by itself, wherein a “triplex structure” is a covalently or non-covalently bound complex of three subunits. As used herein, the term “collagen scaffold domain” refers to the collagenous or collagen-like domains that direct self-trimerization of the scaffold domain.

As used herein, the terms “directed against” or “specifically binds to” a ligand or an epitope are well understood in the art. A molecule is said to exhibit “specific binding” if it reacts more frequently, more rapidly, with greater duration and/or with greater affinity with a particular target ligand or epitope than it does with alternative targets. An antibody fragment or heterologous peptide “specifically binds” to a target ligand or epitope if it binds with greater affinity, avidity, more readily, and/or with greater duration than it binds to other substances. For example, a single-chain antibody fragment (scFv) that specifically (or preferentially) binds to a ligand or epitope therein is a fragment that binds this target antigen with greater affinity, avidity, more readily, and/or with greater duration than it binds to other ligands or other epitopes. It is also understood with this definition that, for example, a heterologous peptide or scFv that specifically binds to a first target ligand or antigen may or may not specifically or preferentially bind to a second target ligand or antigen. As such, “specific binding” or “preferential binding” does not necessarily require (although it can include) exclusive binding.

In some embodiments, an heterologous peptide as described herein has a suitable binding affinity for the target ligand or epitopes thereof. As used herein, “binding affinity” refers to the apparent association constant or K_{A}. The K_{A} is the reciprocal of the dissociation constant (K_{D}). The heterologous peptide described herein may have a binding affinity (K_{D}) of at least 10⁻⁵, 10⁻⁶, 10⁻⁷, 10⁻⁸, 10⁻⁹, 10⁻¹⁰ M, or lower for the target antigen, ligand or antigenic epitope. In a particular example, the heterologous peptides of the present disclosure may have a binding affinity of at least 10⁻⁶ M for a target ligand or epitope. An increased binding affinity corresponds to a decreased K_{D}. Higher affinity binding of a heterologous peptide for a first antigen relative to a second antigen can be indicated by a higher K_{A} (or a smaller numerical value K_{D}) for binding the first ligand or antigen than the K_{A} (or numerical value K_{D}) for binding the second antigen. In such cases, the heterologous peptide has specificity for the first ligand or antigen (e.g., a first protein in a first conformation or mimic thereof) relative to the second ligand or antigen (e.g., the same first protein in a second conformation or mimic thereof; or a second protein).

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH 7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(K_{D}+[Free])

It is not always necessary to make an exact determination of K_{A} or K_{D} though, since sometimes it is sufficient to obtain a quantitative measurement of affinity, e.g., determined using a method such as ELISA or FACS analysis, is proportional to K_{A} or K_{D}, and thus can be used for comparisons, such as determining whether a higher affinity is, e.g., two-fold higher, to obtain a qualitative measurement of affinity, or to obtain an inference of affinity, e.g., by activity in a functional assay, e.g., an in vitro or in vivo assay.

“Disease” means any condition or disorder that damages or interferes with the normal function of a cell, tissue, organ or organism.

As used herein, the term “decoy receptor” refers to a nonsignaling receptors that competes with native signaling receptors in binding ligands or antigens in the cell or on the cell surface. Decoy receptors are unable to initiate downstream immune signaling. Decoy receptors of the invention include, but are not limited to, soluble ectodomains of a transmembrane receptor.

The term “epitope” includes any polypeptide capable of specific binding to an antibody or fragment thereof (e.g. an scFv). In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody or fragment thereof (or an scFv). In certain embodiments, an antibody is said to specifically bind an antigen when it preferentially recognizes its target antigen in a complex mixture of proteins and/or macromolecules.

The term “ligand” includes any polypeptide capable of specific binding to a receptor or fragment thereof (e.g. a transmembrane receptor). In certain embodiments, ligands include chemically active groupings of molecules such as amino acids, sugar side chains, phosphoryl, or sulfonyl, and, in certain embodiments, may have specific three dimensional structural characteristics, and/or specific charge characteristics.

The term “effective amount” includes an amount effective, at dosages and for periods of time necessary, to achieve the desired result, e.g., sufficient to treat a disease or disorder delineated herein. An effective amount of compound of the invention may vary according to factors such as the disease state, age, and weight of the subject, and the ability of the compound of the invention to elicit a desired response in a cell or in the subject. Dosage regimens may be adjusted to provide the optimum therapeutic response. An effective amount is also one in which any toxic or detrimental effects (e.g., side effects) of the compound of the invention are outweighed by the therapeutically beneficial effects.

The term “in combination with” is intended to refer to all forms of administration that provide an a compound of the invention together with an additional pharmaceutical agent, such as a second compound used in clinic for treating or preventing osteoclast-related disease or disorder, where the two are administered concurrently or sequentially in any order.

The terms “isolated,” “purified,” “pure” or “biologically pure” refer to material that is substantially or essentially free from components (such as proteins, nucleic acids, carbohydrates, and other cellular materials) that normally accompany it as found in its native or natural state, e.g., its state in an organism in which the compound or material naturally occurs. Purity and homogeneity are typically determined using analytical chemistry techniques such as polyacrylamide gel electrophoresis or high performance liquid chromatography. In certain embodiments, a compound of this invention is at least 50% pure, 60% pure, 75% pure, 80% pure, 85% pure, at least 90% pure, or at least 95% pure (e.g., by weight). In certain instances, the compound is at least 98% pure, 99% pure, 99.5% pure, 99.8% pure, or 99.9% pure.

The term “subject” includes organisms which are capable of suffering from a disorder as described herein or who could otherwise benefit from the administration of a compound of the present invention, such as human and non-human animals. Preferred humans include human patients suffering from or prone to suffering from diseases or disorders as discussed above, as described herein. Mammalian species that may benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters. A “subject identified as being in need of treatment” includes a subject diagnosed, e.g., by a medical or veterinary professional, as suffering from or susceptible to a disease, disorder or condition described herein.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention.

The term “substantially greater affinity,” as used herein, signifies a measurable increase in the affinity for a disclosed polypeptide as compared with the affinity for known secreted proteins. This greater affinity at least 1.5-fold, 2-fold, 5-fold 10-fold, 100-fold, 10³-fold, 10⁴-fold, 10⁵-fold, 10⁶-fold or greater for a polypeptide of the invention than for known secreted proteins such as members of the TNF-like family of proteins.

As used herein, “Toll Like Receptors (TLRs)” encompass endogenous pattern recognition receptors that recognize exogenous pathogen and endogenous danger associated molecular patterns (PAMPs and DAMPs). TLRs are upregulated in neurodegenerative proteinopathies, such as Alzheimer's disease (AD) and Parkinson's disease (PD), and may lead to excessive inflammatory signaling. Exemplary TLRs include TLR2, TLR4, and TLR5.

The term “effective amount,” as used herein, refers to an amount that is capable of treating or ameliorating a disease or condition or otherwise capable of producing an intended therapeutic effect.

As used herein, the terms “engineered” and “recombinant” cells are intended to refer to a cell into which an exogenous polynucleotide segment (such as DNA segment that leads to the transcription of a biologically active molecule) has been introduced. Therefore, engineered cells are distinguishable from naturally occurring cells, which do not contain a recombinantly introduced exogenous DNA segment. Engineered cells are, therefore, cells that comprise at least one or more heterologous nucleic acid segments introduced through the hand of man.

The term “promoter,” as used herein refers to a region or regions of a nucleic acid sequence that regulates transcription.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “operably linked,” as used herein, refers to the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

As used herein, the term “variant” refers to a molecule (e.g. a CCD protein sequence) having characteristics that deviate from what occurs in nature, e.g., a “variant” is at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. Variants of a protein molecule, e.g. a CCD, may contain modifications to the amino acid sequence (e.g., having 1, 2, 3, 4, 5, 6, 7, 8, 9, 10, 10-15, or 15-20 amino acid substitutions) relative to the wild type protein sequence, which arise from point mutations installed into the nucleic acid sequence encoding the protein. These modifications include chemical modifications as well as truncations, such as truncations at the N- or C-terminus of a protein sequence.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence. The reference sequence may be a subset of a larger sequence.

When highly-homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988) and blastn computer program based on the algorithm of Brutlag et al. (Comp. App. Biosci. 6:237-245 (1990). A preferred method for determining the best overall match between a query sequence (e.g., a sequence of the present disclosure) and a subject sequence, also referred to as a global sequence alignment, can be determined using the FASTA or blastn. In a sequence alignment the query and subject sequences are either both nucleotide sequences or both amino acid sequences. The result of said global sequence alignment is expressed as percent identity. Preferred parameters used in a FASTA amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Whether a nucleotide is matched/aligned is determined by results of the FASTA sequence alignment. This percentage is then subtracted from the percent identity, calculated by the above FASTA program using the specified parameters, to arrive at a final percent identity score. This final percent identity score is what is used for the purposes of the present disclosure.

### DETAILED DESCRIPTION OF THE INVENTION

Aspects of this disclosure relate to novel scaffolds for improved delivery of peptides of interest to a cell, and in particular the delivery of transgenes encoding peptides of interest using a recombinant AAV technique. The novel C1qTNF collagen domain (CCD) scaffolds described herein provide enhanced expression, secretion and stability of the peptides of interest.

In some embodiments, fusion proteins comprising a CCD and one or more heterologous peptides of interest are provided. In exemplary embodiments, polynucleotides encoding these fusion protein are provided. In further embodiments, a disclosed polynucleotide is inserted into a recombinant AAV (rAAV) expression vector (or nucleic acid segment), which may subsequently be packaged into an rAAV particle. In exemplary embodiments, such an rAAV particle is suitable for administration to a cell, tissue or organ, or subject suffering from, diagnosed with, or at risk of having a disease, disorder or condition.

The present disclosure is based in part on the observation that engineered CCD scaffolds used in a monomeric fusion protein co-localize and associate with one another in vitro and in vivo to form multimeric fusion proteins. These multimeric fusion proteins may be dimeric, trimeric, tetrameric, hexameric or octameric. In exemplary embodiments, the disclosed multimeric fusion proteins are hexameric. It was discovered that only a single fusion protein monomer needed to be encoded onto an rAAV nucleic acid vector to achieve sufficient formation of a multimer in vivo due to natural association of collagen domains in cells following rAAV-mediated delivery of a polynucleotide encoding a fusion protein monomer. Accordingly, the present disclosure provides rAAV nucleic acid vectors encoding the monomeric fusion proteins described herein.

In certain embodiments, the disease, disorder or condition that is targeted for treatment by the disclosed methods and compositions is implicated by the activity, localization or binding behavior of an intracellular peptide. The present disclosure provides fusion proteins, and rAAV particles for delivery of fusion proteins, that are designed to treat such diseases. As such, these fusion proteins may include an intracellular heterologous peptide. Exemplary intracellular heterologous peptides include TNF-related apoptosis-inducing ligand (TRAIL), Wnt inhibitory factor 1 (WIF1), soluble toll-like receptors (sTLRs), various scFv's and Inducible T-cell Co-Stimulator (Icos) receptors.

In certain embodiments, the disease, disorder or condition that is targeted for treatment by the disclosed methods and compositions is implicated by the activity or binding behavior of an extracellular, cell-surface or interstitial peptide. The present disclosure provides fusion proteins, and rAAV particles for delivery of fusion proteins, that are designed to treat such diseases. Examples of secreted peptides that play a central role in biological process include cytokines, hormones, extracellular matrix proteins (adhesion molecules), proteases, and growth and differentiation factors. As such, these fusion proteins may include an secretable heterologous peptide. The rAAV particles and compositions of the present disclosure may provide enhanced secretion of these peptides. Exemplary secretable heterologous peptides include soluble ectodomains of various TLRs, Lymphocyte-activation gene 3 (Lag3) and growth factor receptors (e.g. EGFR, VEGFR).

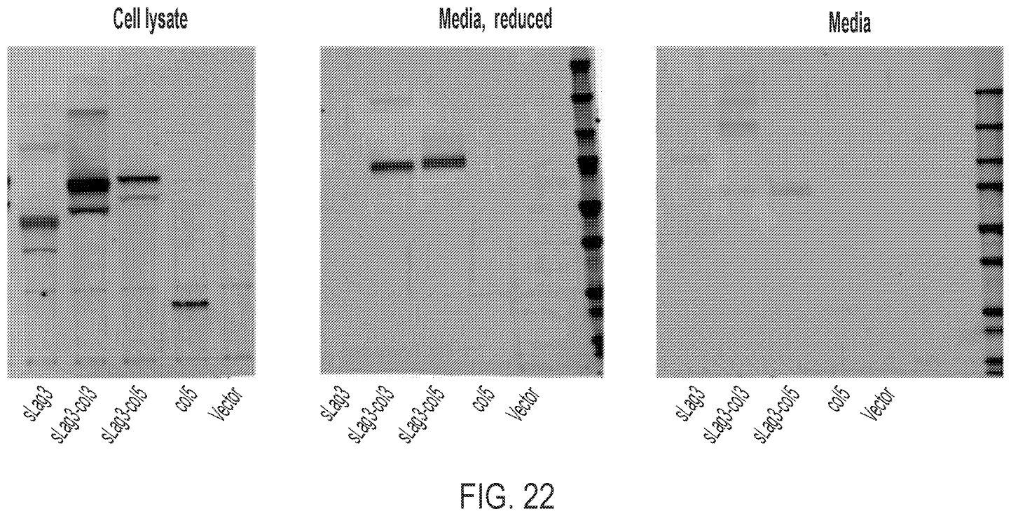

In some embodiments, the CCD from human C1qTNF3 is employed as the CCD scaffold of the disclosed fusion proteins. Among the C1qTNF family members, the C1qTNF3 protein was selected as an exemplary precursor because it has low endogenous expression levels in the human brain. Furthermore, a genetically modified collagen domain from C1qTNF3 outperformed a corresponding domain from C1qTNF5 in an assay evaluating ability of the domains to mediate multimerization while conjugated to a soluble Lymphocyte-activation gene 3 (sLag3) heterologous peptide (see FIG. 22).

In particular embodiments, a variant of the native CCD from human C1qTNF3 is employed as a scaffold. The native CCD from C1qTNF3 is 65 amino acids in length. In some embodiments, the CCD variant is a truncated variant. Exemplary truncated CCDs may have a length of about 65 amino acids, 80 amino acids, 100 amino acids, 115 amino acids, 120 amino acids, 125 amino acids, 130 amino acids, 150 amino acids, 175 amino acids, 200 amino acids, or more than 200 amino acids.

The CCD variant may further comprise a modified amino acid sequence. Disclosed CCD domains may differ relative to a wild-type CCD domain by 1, 2, 3, 4, 5, 6, 7, 8, 9, 10, 10-15, or 15-20 amino acids. These differences relative to the wild-type sequence may encompass truncations at the N- and/or C-terminus of the domain. Exemplary CCDs may be optimized sequences, that is they comprise amino acid substitutions that provide optimal expression or activity in vitro or in vivo without disrupting the Gly-X-Y repeat sequences. An exemplary CCD is the 125-amino acid optimized CCD sequence of SEQ ID NO: 1:

GYQGPPGPPGPPGIPGNHGNNGNNGATGHEGAKGEKGDK
GDLGPRGERGQHGPKGEKGYPGIPGYCGPGPPGPPGIPG
NHGNNGNNGATGHEGAKGEKGDKGDLGPRGERGQHGPKG
EKGYPGIP.

In some embodiments, the CCD domain comprises a sequence having at least 90% sequence identity, at least 95% sequence identity, or at least 99% sequence identity to the amino acid sequence SEQ ID NO: 1. The CCD of SEQ ID NO: 1 comprises a dimer of a 60-amino acid modified Gly-X-Y repeat sequence of the C1qTNF3 protein. This 60-amino acid comprises a consensus sequence, GYQGPPGPPGPPGIPGNHGNNGNNGATGHEGAKGEKGDKGDLGPRGERGQHGPKG EKGYPGIP (SEQ ID NO: 10). In some embodiments, the CCD comprises a sequence having at least 90% sequence identity, at least 95% sequence identity, or at least 99% sequence identity to the amino acid sequence SEQ ID NO: 10. In some embodiments, the fusion proteins of the invention may be more tolerant of mutations in regions of the amino acid sequence other than in a monomer or dimer of SEQ ID NO: 10.

In some embodiments, the CCD is derived from any of the human C1qTNF family members. In some embodiments, the CCD is derived from human C1qTNF5, human C1qTNF1 or human C1qTNF2, or a variant thereof. In particular embodiments, the CCD comprises a sequence having at least 90% sequence identity, at least 95% sequence identity or at least 99% sequence identity to the amino acid sequence of any one of SEQ ID NOs 2-4:

C1qTNF1
(SEQ ID NO: 2)
GEKGDRGDRGLQGKYGKTGSAGARGHTGPKGQKGSMGAP
GERGEKGDRGDRGLQGKYGKTGSAGARGHTGPKGQKGSM
GAPGERGEKGDRGDRGLQGKYGKTGSAGARGHTGPKGQK
GSMGAPGER
C1qTNF2
(SEQ ID NO: 3)
GPQGPPGPPGAPGPSGMMGRMGFPGKDGQDGHDGDRGD
SGEEGPPGRTGNRGKPGPKGKAGAIGRAGPRGPKGVNG
TPGKHGTPGKKGPKGKKGEPGLPGPC.

It was determined that a length of about 110-175 amino acids was optimal for a single CCD monomer in accordance with the disclosed fusion proteins, inclusive of the affinity tag. Accordingly, CCDs derived from C1qTNF family collagen domains (e.g., Gly-X-Y repeat domains) having lengths of 65 amino acids or less were constructed by an oligomerization of two or three copies of the relevant collagen domains to produce sequences of 120-125 amino acids in length. This was the case for the CCDs derived from C1qTNF1, C1qTNF3, C1qTNF5, C1qTNF6, and C1qTNF8.

In other embodiments, the CCD is derived from human C1qTNF5, human C1qTNF6, human C1qTNF7, human C1qTNF8, human C1qTNF9, human C1qTNF9B, or a variant thereof. In particular embodiments, the CCD comprises a sequence having at least 90% sequence identity, at least 95% sequence identity or at least 99% sequence identity to the amino acid sequence of any one of SEQ ID NOs: 4-9:

C1qTNF5
(SEQ ID NO: 4)
GHPGLPGTPGHHGSQGLPGRDGRDGRDGAPGAPGE
KGEGGRPGLPGPRGDPGPRGEAGPAGPTGPAGECG
HPGLPGTPGHHGSQGLPGRDGRDGRDGAPGAPGEK
GEGGRPGLPGPRGDPGPRGEAGPAGPTGPAGEC
C1qTNF6
(SEQ ID NO: 5)
GDKGDPGPMGLPGYMGREGPQGEPGPQGSKGDKGE
MGSPGAPGDKGDPGPMGLPGYMGREGPQGEPGPQG
SKGDKGEMGSPGAPGDKGDPGPMGLPGYMGREGPQ
GEPGPQGSKGDKGEMGSPGAP
C1qTNF7
(SEQ ID NO: 6)
GLPGPPGPPGANGSPGPHGRIGLPGRDGRDGRKGE
KGEKGTAGLRGKTGPLGLAGEKGDQGETGKKGPIG
PEGEKGEVGPIGPPGPKGDRGEQGDPGLPGVCRCG
SI
C1qTNF8
(SEQ ID NO: 7)
GEKGEAGVRGRAGRSGKEGPPGARGLQGRRGQKGQ
VGPPGAAGEKGEAGVRGRAGRSGKEGPPGARGLQG
RRGQKGQVGPPGAAGEKGEAGVRGRAGRSGKEGPP
GARGLQGRRGQKGQVGPPGAA
C1qTNF9
(SEQ ID NO: 8)
GHPGIPGNPGHNGLPGRDGRDGAKGDKGDAGEPGR
PGSPGKDGTSGEKGERGADGKVEAKGIKGDQGSRG
SPGKHGPKGLAGPMGEKGLRGETGPQGQKGNKGDV
GPTGPEGPRGNIGPLGPTGLPGPMGPIGKPGPKGE
AGPTGPQGEPGVRGIRGWKGDRGEKGKIGET
C1qTNF9B
(SEQ ID NO: 9)
GHPGIPGNPGHNGLPGRDGRDGAKGDKGDAGEPGC
PGSPGKDGTSGEKGERGADGKVEAKGIKGDQGSRG
SPGKHGPKGLAGPMGEKGLRGETGPQGQKGNKGDV
GPTGPEGPRGNIGPLGPTGLPGPMGPIGKPGPKGE
AGPTGPQGEPGVRGIRGWKGDRGEKGKIGET.

The first and/or second heterologous peptides of the fusion proteins described herein may be any small peptide. The first and/or second heterologous peptides may comprise a mouse (Mus musculus) or human peptide. The first and/or second heterologous peptides may comprise a single-chain variable fragment (scFv), an immune checkpoint modulator, a soluble lymphocyte activation gene (sLag), a growth factor receptor, or an ectodomain or variant thereof.

The heterologous peptides of the fusion proteins may comprise an immune checkpoint-activating peptide, an immune checkpoint-inactivating peptide, a Type II transmembrane protein, a receptor inhibiting protein (RIP) kinase inhibitor, an extracellular matrix metalloproteinase inhibitor, or a soluble peptide, e.g. a soluble peptide without a transmembrane domain.

In some embodiments, at least one of the first or second heterologous peptides is selected from the group consisting of Lymphocyte-activation gene 3 (LAG3), Programmed Cell Death 1 (PD-1), Wnt inhibitory factor 1 (WIF1), Inducible T-cell Co-Stimulator (Icos), Notch homolog 1 (NOTCH1), Vascular endothelial growth factor receptor 1 (VEGFR1), or Fibroblast growth factor receptor 1 (FGFR1), or an ectodomain or variant thereof. In some embodiments, at least one of the first or second heterologous peptides is selected from the group consisting of glucocerebrosidase (GBA), suicide gene HSV-TK, the HSV-TK SR39 mutant, or a variant thereof.

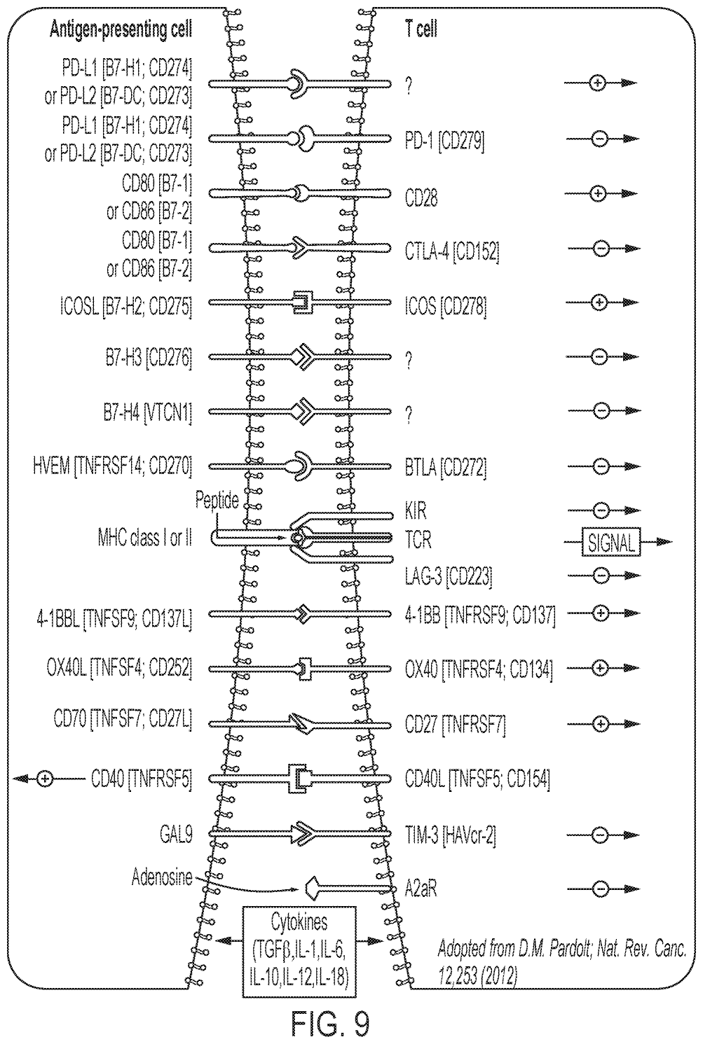

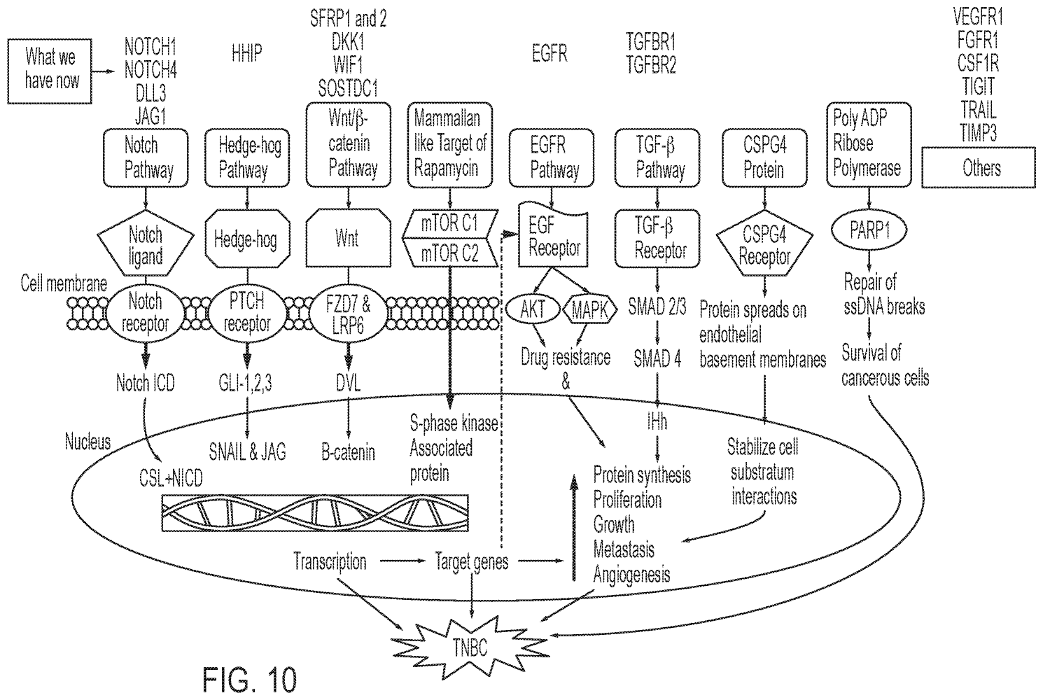

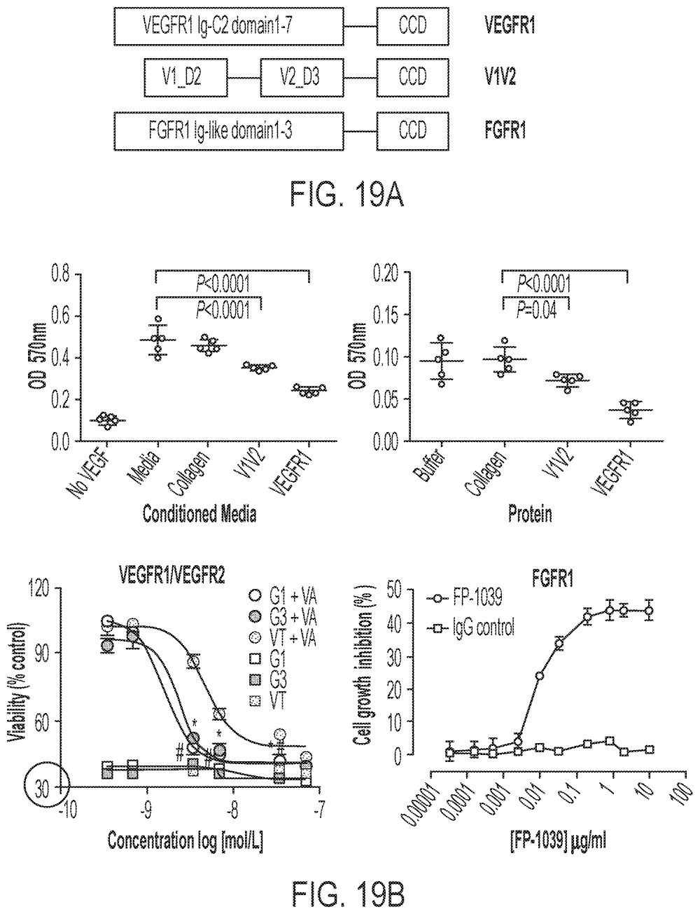

In other embodiments, at least one of the first or second heterologous peptides is selected from the group consisting of Notch homolog 4 (NOTCH4), Cytotoxic T-lymphocyte-associated protein 4 (CTLA4), Delta-like 3 (DLL3), Jagged1 (JAG1), Cluster of Differentiation 276 (B7H3), Epidermal growth factor receptor (EGFR), Transforming growth factor beta receptor I (TGFBR1), Transforming growth factor, beta receptor II (TGFBR2), Hedgehog interacting protein (HHIP), Secreted frizzled-related protein 1 (SFRP1), Secreted frizzled-related protein 2 (SFRP2), Dickkopf-related protein 1 (DKK1), Sclerostin domain-containing protein 1 (SOSTDC1), Colony stimulating factor 1 receptor (CSF1R), T cell immunoreceptor with Ig and ITIM domains (TIGIT), TNF-related apoptosis-inducing ligand (TRAIL), Metalloproteinase inhibitor 3 (TIMP3), Hepatitis A virus cellular receptor 2 (HAVCR2), a VEGFR1 domain 2, a VEGFR2 domain 3, and a soluble toll-like receptor (sTLR, e.g., sTLR5), or an ectodomain or variant thereof. Schematics showing exemplary fusion proteins containing heterologous peptides of interest as disclosed herein are illustrated in FIGS. 9, 10 and 19A.

In certain embodiments, the fusion protein includes a domain corresponding to a merozoite surface protein (“MSP”), a protein anchored to the cell surface via a glycosylphosphatidylinositol (GPI) moiety. MSPs are derived from the malaria parasite Plasmodium falciparum. In some embodiments, the fusion protein comprises both an MSP and a CCD. In other embodiments, the fusion protein contains an MSP and does not contain a CCD.

In particular embodiments, the disclosed fusion proteins comprise an MSP sequence that comprises the following amino acid sequence: DGIFCSSSNFLGISFLLILMLILYSFI (SEQ ID NO: 49). In some embodiments, the disclosed fusion proteins comprise an amino acid sequence that is at least 85%, at least 90%, at least 95%, at least 98%, at least 99%, or at least 99.5% identical to the sequence of SEQ ID NO: 49. In certain embodiments, the fusion proteins comprise an MSP sequence at the C-terminus. In other embodiments, the MSP sequence may be positioned at the N terminus.

In various embodiments, the heterologous peptide is a single chain antibody fragment (scFv), which may comprise only one variable region (e.g., VH) or comprise both a VH and a VL. In particular embodiments, a first heterologous peptide comprises an scFv having affinity for a tau protein (α-tau) or an scFv having affinity for an amyloid beta peptide (α-Aβ). In other embodiments, the heterologous peptide comprises a B11 scFv with affinity to VEGFR-2. See Boldicke et al., Stem Cells. 2001, 19(1):24-36, herein incorporated by reference.

In some embodiments, the disclosed fusion proteins comprise i) a CCD, ii) a heterologous peptide, and iii) one or more affinity tags, such as a FLAG tag or a polyhistidine (His₆) tag. In particular embodiments, In particular embodiments, the fusion proteins comprise one or two His₆ tags. In particular embodiments, the fusion proteins comprise a His₆ tag at the C-terminus of the protein. In particular embodiments, the fusion proteins comprise a His₆ tag positioned C-terminal of the heterologous protein.

In other embodiments, the fusion proteins comprise one or two FLAG tags. In particular embodiments, the fusion proteins comprise a FLAG tag positioned C-terminal of the heterologous protein. In particular embodiments, the fusion protein has a FLAG tag sequence comprising DYKDDDDK (SEQ ID NO: 12).

In some embodiments, the fusion proteins of the disclosure comprise a CCD having a FLAG affinity tag at the C terminus of the CCD. Accordingly, in particular embodiments, the fusion proteins comprise a CCD with a sequence having at least 90% sequence identity, at least 95% sequence identity or at least 99% sequence identity to the amino acid sequence of SEQ ID NO: 11 below (the FLAG tag is indicated in underlined italics):

MGYQGPPGPPGPPGIPGNHGNNGNNGATGHEGAKGEKGDKGD
LGPRGERGQHGPKGEKGYPGIPGYCGPGPPGPPGIPGNHGNN
GNNGATGHEGAKGEKGDKGDLGPRGERGQHGPKGEKGYPGIP
DYKDDDDKLQISSTVAAARV.

In some embodiments, the fusion proteins of the disclosure comprise a CCD having a FLAG affinity tag at the C terminus of the CCD. In certain embodiments, these fusion proteins further comprise a poly(A) tail sequence. Accordingly, in particular embodiments, the fusion proteins comprise a CCD having a sequence having at least 90% sequence identity, at least 95% sequence identity or at least 99% sequence identity to the amino acid sequence of SEQ ID NO: 109 below (the FLAG tag is indicated in underlined italics and the poly(A) tail and His₆ tag are indicated in bold underline):

MGYQGPPGPPGPPGIPGNHGNNGNNGATGHEGAKGEKGDKGD
LGPRGERGQHGPKGEKGYPGIPGYCGPPGPPGPPGIPGNHGN
NGNNGATGHEGAKGEKGDKGDLGPRGERGQHGPKGEKGYPGI
PDYKDDDDKLQISSTV [symbol] V

In certain embodiments, the fusion proteins further comprise a secretion signal sequence. In some embodiments, the fusion proteins (e.g., the heterologous peptides of the fusion proteins) have bispecificity for two or more ligands.

Exemplary fusion proteins that comprise heterologous proteins comprising immune checkpoint modulators are described herein. Unless otherwise indicated in the fusion protein name, the CCD is positioned at the C-terminus of the protein. Where indicated with a “-MSP” in the fusion protein name, the fusion protein does not contain a CCD, but rather contains an MSP sequence that is positioned at the C-terminus of the protein.

In various embodiments, the disclosed fusion proteins comprise an amino acid sequence that is at least 85%, at least 90%, at least 95%, at least 98%, at least 99%, or at least 99.5% identical to any of the amino acid sequences that follow (SEQ ID NOs: 13-102 and 105-108). Any of the disclosed fusion proteins may differ relative to any of the following amino acid sequences by 1, 2, 3, 4, 5, 6, 7, 8, 9, 10, 10-15, 15-20, 20-30, 30-50, or more than 50 amino acids. These differences may comprise amino acids that have been inserted, deleted, or substituted relative to the sequence of any of SEQ ID NOs: 13-64 or 105-108. In some embodiments, the disclosed fusion proteins contain stretches of about 10, about 20, about 25, about 40, 50, about 75, about 100, about 125, about 150, about 175, or about 180 amino acids in common with the sequence of any of SEQ ID NOs: 13-64 or 105-108. In some embodiments, the disclosed fusion proteins comprise truncations at the 5′ or 3′ end relative to any of SEQ ID NOs: 13-64 or 105-108.

In various embodiments, the disclosed fusion proteins comprise an amino acid sequence that is at least 85%, at least 90%, at least 95%, at least 98%, at least 99%, or at least 99.5% identical to any of the amino acid sequences that follow (SEQ ID NOs: 13-102 and 105-108) in regions other than the consensus amino acid sequence of SEQ ID NO: 10 or the dimer of this consensus sequence set forth as SEQ ID NO: 1. In some embodiments, the disclosed fusion proteins comprise stretches of about 50, about 75, about 100, about 125, about 150, about 175, about 200, about 250, about 300, about 350, or about 375 amino acids in common with the sequences of any of SEQ ID NOs: 13-102 and 105-108. In some embodiments, these stretches in common include the consensus amino acid sequence of SEQ ID NO: 10 or the dimer of this consensus sequence set forth as SEQ ID NO: 1.

Many of the sequences that follow (SEQ ID NOs: 13-102 and 105-108) are illustrated with poly(A) tails and polyhistidine tags, FLAG tags, or other tags, at the C terminus. However, as can readily be visualized by one skilled in the art, in some embodiments, the disclosed exemplary fusion proteins do not contain any one of these tags. In some embodiments, any of the proteins set forth in SEQ ID NOs: 13-102 and 105-108 comprise a tag other than a polyhistidine tag or a FLAG tag. In some embodiments, the polyhistidine tag or FLAG tag of any of the proteins of SEQ ID NOs: 13-102 and 105-108 is substituted with an alternative tag, such as a myc-tag, Strep-tag, E-tag, hemagglutin tag, T7 tag, S-tag, HSV, VSV-G, anti-Xpress, and VS-tag.

(“ms” designates mouse sequences, and “hs” designates human sequences):

CCD-Cluster of differentiation 40
ligand (msCd40L)
(SEQ ID NO: 13)
METDTLLLWVLLLWVPGSTGDLKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISVNLHEDFVFIK
KLKRCNKGEGSLSLLNCEEMRRQFEDLVKDITLNK
EEKKENSFEMQRGDEDPQIAAHVVSEANSNAASVL
QWAKKGYYTMKSNLVMLENGKQLTVKREGLYYVYT
QVTFCSNREPSSQRPFIVGLWLKPSSGSERILLKA
ANTHSSSQLCEQQSVHLGGVFELQAGASVFVNVTE
ASQVIHRVGFSSFGLLKLAAAHHHHHHV
CCD-Cluster of differentiation 70
(msCD70)
(SEQ ID NO: 14)
METDTLLLWVLLLWVPGSTGDLKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISHPEPHTAELQL
NLTVPRKDPTLRWGAGPALGRSFTHGPELEEGHLR
IHQDGLYRLHIQVTLANCSSPGSTLQHRATLAVGI
CSPAAHGISLLRGRFGQDCTVALQRLTYLVHGDVL
CTNLTLPLLPSRNADETFFGVQWICPAAAHHHHHH
V
CCD-msCD70
(SEQ ID NO: 15)
METDTLLLWVLLLWVPGSTGDLKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISHPEPHTAELQL
NLTVPRKDPTLRWGAGPALGRSFTHGPELEEGHLR
IHQDGLYRLHIQVTLANCSSPGSTLQHRATLAVGI
CSPAAHGISLLRGRFGQDCTVALQRLTYLVHGDVL
CTNLTLPLLPSRNADETFFGVQWICPAAAHHHHHH
V
CCD-CD137 ligand (msCD137L)
(SEQ ID NO: 16)
METDTLLLWVLLLWVPGSTGDLKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISITTSPNLGTRE
NNADQVTPVSHIGCPNTTQQGSPVFAKLLAKNQAS
LCNTTLNWHSQDGAGSSYLSQGLRYEEDKKELVVD
SPGLYYVFLELKLSPTFTNTGHKVQGWVSLVLQAK
PQVDDFDNLALTVELFPCSMENKLVDRSWSQLLLL
KAGHRLSVGLRAYLHGAQDAYRDWELSYPNTTSFG
LFLVKPDNPWEAAAHHHHHHV
CCD-Herpesvirus entry mediator
ligand (msHveml)
(SEQ ID NO: 17)
METDTLLLWVLLLWVPGSTGDLKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISHLPDGGKGSWE
KLIQDQRSHQANPAAHLTGANASLIGIGGPLLWET
RLGLAFLRGLTYHDGALVTMEPGYYYVYSKVQLSG
VGCPQGLANGLPITHGLYKRTSRYPKELELLVSRR
SPCGRANSSRVWWDSSFLGGVVHLEAGEEVVVRVP
GNRLVRPRDGTRSYFGAFMVAAAHHHHHHV
CCD-msOx40L, or msCD252
(SEQ ID NO: 18)
METDTLLLWVLLLWVPGSTGDLKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISIQRLRGAVTRC
EDGQLFISSYKNEYQTMEVQNNSVVIKCDGLYIIY
LKGSFFQEVKIDLHFREDHNPISIPMLNDGRRIVF
TVVASLAFKDKVYLTVNAPDTLCEHLQINDGELIV
VQLTPGYCAPEGSYHSTVNQVPLAAAHHHHHHV
Cluster of Differentiation 276
(msB7H3, or CD276)
(SEQ ID NO: 19)
MLRGWGGPSVGVCVRTALGVLCLCLTGAVEVQVSE
DPVVALVDTDATLRCSFSPEPGFSLAQLNLIWQLT
DTKQLVHSFTEGRDQGSAYSNRTALFPDLLVQGNA
SLRLQRVRVTDEGSYTCFVSIQDFDSAAVSLQVAA
PYSKPSMTLEPNKDLRPGNMVTITCSSYQGYPEAE
VFWKDGQGVPLTGNVTTSQMANERGLFDVHSVLRV
VLGANGTYSCLVRNPVLQQDAHGSVTITGLKEFMG
YQGPPGPPGPPGIPGNHGNNGNNGATGHEGAKGEK
GDKGDLGPRGERGQHGPKGEKGYPGIPGYCGPPGP
PGPPGIPGNHGNNGNNGATGHEGAKGEKGDKGDLG
PRGERGQHGPKGEKGYPGIPDYKDDDDKLQISSTV
AAAHHHHHHV
V-set domain-containing T-cell
activation inhibitor 1
(msB7h4, or VTCN1)
(SEQ ID NO: 20)
MASLGQIIFWSIINIIIILAGAIALIIGFGISGKH
FITVTTFTSAGNIGEDGTLSCTFEPDIKLNGIVIQ
WLKEGIKGLVHEFKEGKDDLSQQHEMFRGRTAVFA
DQVVVGNASLRLKNVQLTDAGTYTCYIRTSKGKGN
ANLEYKTGAFSMPEINVDYNASSESLRCEAPRWFP
QPTVAWASQVDQGANFSEVSNTSFELNSENVTMKV
VSVLYNVTINNTYSCMIENDIAKATGDIKVTDSEV
KRRLKEFMGYQGPPGPPGPPGIPGNHGNNGNNGAT
GHEGAKGEKGDKGDLGPRGERGQHGPKGEKGYPGI
PGYCGPPGPPGPPGIPGNHGNNGNNGATGHEGAKG
EKGDKGDLGPRGERGQHGPKGEKGYPGIPDYKDDD
DKLQISSTVAAAHHHHHHV
B- and T-lymphocyte attenuator
(msBTLA)
(SEQ ID NO: 21)
MKTVPAMLGTPRLFREFFILHLGLWSILCEKATKR
NDEECPVQLTITRNSKQSARTGELFKIQCPVKYCV
HRPNVTWCKHNGTICVPLEVSPQLYTSWEENQSVP
VFVLHFKPIHLSDNGSYSCSTNFNSQVINSHSVTI
HVTERTQNSSEHPLITVSDIPDATNASGPSTMEEL
KEFMGYQGPPGPPGPPGIPGNHGNNGNNGATGHEG
AKGEKGDKGDLGPRGERGQHGPKGEKGYPGIPGYC
GPPGPPGPPGIPGNHGNNGNNGATGHEGAKGEKGD
KGDLGPRGERGQHGPKGEKGYPGIPDYKDDDDKLQ
ISSTVAAAHHHHHHV
msCD27
(SEQ ID NO: 22)
MAWPPPYWLCMLGTLVGLSATLAPNSCPDKHYWTG
GGLCCRMCEPGTFFVKDCEQDRTAAQCDPCIPGTS
FSPDYHTRPHCESCRHCNSGFLIRNCTVTANAECS
CSKNWQCRDQECTECDPPLNPALTRQPSETPSPQP
PPTHLPHGTEKPSWPLHRQLPNSTVYSQRSSHRLK
EFMGYQGPPGPPGPPGIPGNHGNNGNNGATGHEGA
KGEKGDKGDLGPRGERGQHGPKGEKGYPGIPGYCG
PPGPPGPPGIPGNHGNNGNNGATGHEGAKGEKGDK
GDLGPRGERGQHGPKGEKGYPGIPDYKDDDDKLQI
SSTVAAAHHHHHHV
msCD28
(SEQ ID NO: 23)
MTLRLLFLALNFFSVQVTENKILVKQSPLLVVDSN
EVSLSCRYSYNLLAKEFRASLYKGVNSDVEVCVGN
GNFTYQPQFRSNAEFNCDGDFDNETVTFRLWNLHV
NHTDIYFCKIEFMYPPPYLDNERSNGTIIHIKEKH
LLKEFMGYQGPPGPPGPPGIPGNHGNNGNNGATGH
EGAKGEKGDKGDLGPRGERGQHGPKGEKGYPGIPG
YCGPPGPPGPPGIPGNHGNNGNNGATGHEGAKGEK
GDKGDLGPRGERGQHGPKGEKGYPGIPDYKDDDDK
LQISSTVAAAHHHHHHV
msCD40
(SEQ ID NO: 24)
MVSLPRLCALWGCLLTAVHLGQCVTCSDKQYLHDG
QCCDLCQPGSRLTSHCTALEKTQCHPCDSGEFSAQ
WNREIRCHQHRHCEPNQGLRVKKEGTAESDTVCTC
KEGQHCTSKDCEACAQHTPCIPGFGVMEMATETTD
TVCHPCPVGFFSNQSSLFEKCYPWTSCEDKNLEVL
QKGTSQTNVLKEFMGYQGPPGPPGPPGIPGNHGNN
GNNGATGHEGAKGEKGDKGDLGPRGERGQHGPKGE
KGYPGIPGYCGPPGPPGPPGIPGNHGNNGNNGATG
HEGAKGEKGDKGDLGPRGERGQHGPKGEKGYPGIP
DYKDDDDKLQISSTVAAAHHHHHHV
msCD80
(SEQ ID NO: 25)
MACNCQLMQDTPLLKFPCPRLILLFVLLIRLSQVS
SDVDEQLSKSVKDKVLLPCRYNSPHEDESEDRIYW
QKHDKVVLSVIAGKLKVWPEYKNRTLYDNTTYSLI
ILGLVLSDRGTYSCVVQKKERGTYEVKHLALVKLS
IKADFSTPNITESGNPSADTKRITCFASGGFPKPR
FSWLENGRELPGINTTISQDPESELYTISSQLDFN
TTRNHTIKCLIKYGDAHVSEDFTWEKPLKEFMGYQ
GPPGPPGPPGIPGNHGNNGNNGATGHEGAKGEKGD
KGDLGPRGERGQHGPKGEKGYPGIPGYCGPPGPPG
PPGIPGNHGNNGNNGATGHEGAKGEKGDKGDLGPR
GERGQHGPKGEKGYPGIPDYKDDDDKLQISSTVAA
AHHHHHHV
msCD86
(SEQ ID NO: 26)
MDPRCTMGLAILIFVTVLLISDAVSVETQAYFNGT
AYLPCPFTKAQNISLSELVVFWQDQQKLVLYEHYL
GTEKLDSVNAKYLGRTSFDRNNWTLRLHNVQIKDM
GSYDCFIQKKPPTGSIILQQTLTELSVIANFSEPE
IKLDQNVTGNSGINLTCMSKQGHPKPKKMYFLITN
STNEYGDNMQISQDNVTELFSISNSLSLSFPDGVW
HMTVVCVLETESMKISSKPLNFTQELKEFMGYQGP
PGPPGPPGIPGNHGNNGNNGATGHEGAKGEKGDKG
DLGPRGERGQHGPKGEKGYPGIPGYCGPPGPPGPP
GIPGNHGNNGNNGATGHEGAKGEKGDKGDLGPRGE
RGQHGPKGEKGYPGIPDYKDDDDKLQISSTVAAAH
HHHHHV
Tumor necrosis factor receptor
superfamily member 9 (msCd137 or
TNFRSF9)
(SEQ ID NO: 27)
MGNNCYNVVVIVLLLVGCEKVGAVQNSCDNCQPGT
FCRKYNPVCKSCPPSTFSSIGGQPNCNICRVCAGY
FRFKKFCSSTHNAECECIEGFHCLGPQCTRCEKDC
RPGQELTKQGCKTCSLGTFNDQNGTGVCRPWTNCS
LDGRSVLKTGTTEKDVVCGPPVVSFSPSTTISVTP
EGGLKEFMGYQGPPGPPGPPGIPGNHGNNGNNGAT
GHEGAKGEKGDKGDLGPRGERGQHGPKGEKGYPGI
PGYCGPPGPPGPPGIPGNHGNNGNNGATGHEGAKG
EKGDKGDLGPRGERGQHGPKGEKGYPGIPDYKDDD
DKLQISSTVAAAHHHHHHV
Cytotoxic T-lymphocyte-associated
protein 4 (msCtla4)
(SEQ ID NO: 28)
MACLGLRRYKAQLQLPSRTWPFVALLTLLFIPVFS
EAIQVTQPSVVLASSHGVASFPCEYSPSHNTDEVR
VTVLRQTNDQMTEVCATTFTEKNTVGFLDYPFCSG
TFNESRVNLTIQGLRAVDTGLYLCKVELMYPPPYF
VGMGNGTQIYVILKEFMGYQGPPGPPGPPGIPGNH
GNNGNNGATGHEGAKGEKGDKGDLGPRGERGQHGP
KGEKGYPGIPGYCGPPGPPGPPGIPGNHGNNGNNG
ATGHEGAKGEKGDKGDLGPRGERGQHGPKGEKGYP
GIPDYKDDDDKLQISSTVAAAHHHHHHV
Hepatitis A virus cellular
receptor 2 (msHavcr2)
(SEQ ID NO: 29)
MFSGLTLNCVLLLLQLLLARSLEDGYKVEVGKNAY
LPCSYTLPTSGTLVPMCWGKGFCPWSQCTNELLRT
DERNVTYQKSSRYQLKGDLNKGDVSLIIKNVTLDD
HGTYCCRIQFPGLMNDKKLELKLDIKAAKVTPAQT
AHGDSTTASPRTLTTERNGSETQTLVTLHNNNGTK
ISTWADEIKLKEFMGYQGPPGPPGPPGIPGNHGNN
GNNGATGHEGAKGEKGDKGDLGPRGERGQHGPKGE
KGYPGIPGYCGPPGPPGPPGIPGNHGNNGNNGATG
HEGAKGEKGDKGDLGPRGERGQHGPKGEKGYPGIP
DYKDDDDKLQISSTVAAAHHHHHHV
Herpesvirus entry mediator (msHvem)
(SEQ ID NO: 30)
MEPLPGWGSAPWSQAPTDNTFRLVPCVFLLNLLQR
ISAQPSCRQEEFLVGDECCPMCNPGYHVKQVCSEH
TGTVCAPCPPQTYTAHANGLSKCLPCGVCDPDMGL
LTWQECSSWKDTVCRCIPGYFCENQDGSHCSTCIQ
HTTCPPGQRVEKRGTHDQDTVCADCLTGTFSLGGT
QEECLPWTNCSAFQQEVRRGTNLKEFMGYQGPPGP
PGPPGIPGNHGNNGNNGATGHEGAKGEKGDKGDLG
PRGERGQHGPKGEKGYPGIPGYCGPPGPPGPPGIP
GNHGNNGNNGATGHEGAKGEKGDKGDLGPRGERGQ
HGPKGEKGYPGIPDYKDDDDKLQISSTVAAAHHHH
HHV
Inducible T-cell costimulator
(msIcos)
(SEQ ID NO: 31)
MKPYFCRVFVFCFLIRLLTGEINGSADHRMFSFHN
GGVQISCKYPETVQQLKMRLFREREVLCELTKTKG
SGNAVSIKNPMLCLYHLSNNSVSFFLNNPDSSQGS
YYFCSLSIFDPPPFQERNLSGGYLHIYESQLKEFM
GYQGPPGPPGPPGIPGNHGNNGNNGATGHEGAKGE
KGDKGDLGPRGERGQHGPKGEKGYPGIPGYCGPPG
PPGPPGIPGNHGNNGNNGATGHEGAKGEKGDKGDL
GPRGERGQHGPKGEKGYPGIPDYKDDDDKLQISST
VAAAHHHHHHV
Inducible T-cell costimulator
ligand (msIcosL)
MQLKCPCFVSLGTRQPVWKKLHVSSGFFSGLGLFL
LLLSSLCAASAETEVGAMVGSNVVLSCIDPHRRHF
(SEQ ID NO: 32)
NLSGLYVYWQIENPEVSVTYYLPYKSPGINVDSSY
KNRGHLSLDSMKQGNFSLYLKNVTPQDTQEFTCRV
FMNTATELVKILEEVVRLRVAANFSTPVISTSDSS
NPGQERTYTCMSKNGYPEPNLYWINTTDNSLIDTA
LQNNTVYLNKLGLYDVISTLRLPWTSRGDVLCCVE
NVALHQNITSISQAESFTGNNTKLKEFMGYQGPPG
PPGPPGIPGNHGNNGNNGATGHEGAKGEKGDKGDL
GPRGERGQHGPKGEKGYPGIPGYCGPPGPPGPPGI
PGNHGNNGNNGATGHEGAKGEKGDKGDLGPRGERG
QHGPKGEKGYPGIPDYKDDDDKLQISSTVAAAHHH
HHHV
Lymphocyte Activation Gene 3 (msLag3)
(SEQ ID NO: 33)
MREDLLLGFLLLGLLWEAPVVSDYKDDDDKSGPGK
ELPVVWAQEGAPVHLPCSLKSPNLDPNFLRRGGVI
WQHQPDSGQPTPIPALDLHQGMPSPRQPAPGRYTV
LSVAPGGLRSGRQPLHPHVQLEERGLQRGDFSLWL
RPALRTDAGEYHATVRLPNRALSCSLRLRVGQASM
IASPSGVLKLSDWVLLNCSFSRPDRPVSVHWFQGQ
NRVPVYNSPRHFLAETFLLLPQVSPLDSGTWGCVL
TYRDGFNVSITYNLKVLGLEPVAPLTVYAAEGSRV
ELPCHLPPGVGTPSLLIAKWTPPGGGPELPVAGKS
GNFTLHLEAVGLAQAGTYTCSIHLQGQQLNATVTL
AVITVTPKSFGLPGSRGKLLCEVTPASGKERFVWR
PLNNLSRSCPGPVLEIQEARLLAERWQCQLYEGQR
LLGATVYAAESSSGAHSARRILKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISSTVAAAHHHHH
HV
Tumor necrosis factor receptor
superfamily member 4 (msOx40,
or TNFRSF4)
(SEQ ID NO: 34)
MYVWVQQPTALLLLGLTLGVTARRLNCVKHTYPSG
HKCCRECQPGHGMVSRCDHTRDTLCHPCETGFYNE
AVNYDTCKQCTQCNHRSGSELKQNCTPTQDTVCRC
RPGTQPRQDSGYKLGVDCVPCPPGHFSPGNNQACK
PWTNCTLSGKQTRHPASDSLDAVCEDRSLLATLLW
ETQRPTFRPTTVQSTTVWPRTSELPSPLKEFMGYQ
GPPGPPGPPGIPGNHGNNGNNGATGHEGAKGEKGD
KGDLGPRGERGQHGPKGEKGYPGIPGYCGPPGPPG
PPGIPGNHGNNGNNGATGHEGAKGEKGDKGDLGPR
GERGQHGPKGEKGYPGIPDYKDDDDKLQISSTVAA
AHHHHHHV
Programmed Cell Death Protein 1
(msPD1)
(SEQ ID NO: 35)
MWVRQVPWSFTWAVLQLSWQSGWLLEVPNGPWRSL
TFYPAWLTVSEGANATFTCSLSNWSEDLMLNWNRL
SPSNQTEKQAAFCNGLSQPVQDARFQIIQLPNRHD
FHMNILDTRRNDSGIYLCGAISLHPKAKIEESPGA
ELVVTERILETSTRYPSPSPLKEFMGYQGPPGPPG
PPGIPGNHGNNGNNGATGHEGAKGEKGDKGDLGPR
GERGQHGPKGEKGYPGIPGYCGPPGPPGPPGIPGN
HGNNGNNGATGHEGAKGEKGDKGDLGPRGERGQHG
PKGEKGYPGIPDYKDDDDKLQISSTVAAAHHHHHH
V
Programmed Cell Death Ligand 1
(msPD-L1)
(SEQ ID NO: 36)
MRIFAGIIFTACCHLLRAFTITAPKDLYVVEYGSN
VTMECRFPVERELDLLALVVYWEKEDEQVIQFVAG
EEDLKPQHSNFRGRASLPKDQLLKGNAALQITDVK
LQDAGVYCCIISYGGADYKRITLKVNAPYRKINQR
ISVDPATSEHELICQAEGYPEAEVIWTNSDHQPVS
GKRSVTTSRTEGMLLNVTSSLRVNATANDVFYCTF
WRSQPGQNHTAELIIPELPALKEFMGYQGPPGPPG
PPGIPGNHGNNGNNGATGHEGAKGEKGDKGDLGPR
GERGQHGPKGEKGYPGIPGYCGPPGPPGPPGIPGN
HGNNGNNGATGHEGAKGEKGDKGDLGPRGERGQHG
PKGEKGYPGIPDYKDDDDKLQISSTVAAAHHHHHH
V
Programmed Cell Death Ligand 2 (msPD-L2)
(SEQ ID NO: 37)
MLLLLPILNLSLQLHPVAALFTVTAPKEVYTVDVG
SSVSLECDFDRRECTELEGIRASLQKVENDTSLQS
ERATLLEEQLPLGKALFHIPSVQVRDSGQYRCLVI
CGAAWDYKYLTVKVKASYMRIDTRILEVPGTGEVQ
LTCQARGYPLAEVSWQNVSVPANTSHIRTPEGLYQ
VTSVLRLKPQPSRNFSCMFWNAHMKELTSAIIDPL
SRLKEFMGYQGPPGPPGPPGIPGNHGNNGNNGATG
HEGAKGEKGDKGDLGPRGERGQHGPKGEKGYPGIP
GYCGPPGPPGPPGIPGNHGNNGNNGATGHEGAKGE
KGDKGDLGPRGERGQHGPKGEKGYPGIPDYKDDDD
KLQISSTVAAAHHHHHHV
Galectin-9 (hLGALS9)
(SEQ ID NO: 38)
MAFSGSQAPYLSPAVPFSGTIQGGLQDGLQITVNG
TVLSSSGTRFAVNFQTGFSGNDIAFHFNPRFEDGG
YVVCNTRQNGSWGPEERKTHMPFQKGMPFDLCFLV
QSSDFKVMVNGILFVQYFHRVPFHRVDTISVNGSV
QLSYISFQPPGVWPANPAPITQTVIHTVQSAPGQM
FSTPAIPPMMYPHPAYPMPFITTILGGLYPSKSIL
LSGTVLPSAQRFHINLCSGNHIAFHLNPRFDENAV
VRNTQIDNSWGSEERSLPRKMPFVRGQSFSVWILC
EAHCLKVAVDGQHLFEYYHRLRNLPTINRLEVGGD
IQLTHVQTLKEFMGYQGPPGPPGPPGIPGNHGNNG
NNGATGHEGAKGEKGDKGDLGPRGERGQHGPKGEK
GYPGIPGYCGPPGPPGPPGIPGNHGNNGNNGATGH
EGAKGEKGDKGDLGPRGERGQHGPKGEKGYPGIPD
YKDDDDKLQISSTVAAAHHHHHHV
CCD-Cluster of differentiation
40 ligand (hsCD40L)
(SEQ ID NO: 39)
METDTLLLWVLLLWVPGSTGDLKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISNLHEDFVFMKT
IQRCNTGERSLSLLNCEEIKSQFEGFVKDIMLNKE
ETKKENSFEMQKGDQNPQIAAHVISEASSKTTSVL
QWAEKGYYTMSNNLVTLENGKQLTVKRQGLYYIYA
QVTFCSNREASSQAPFIASLCLKSPGRFERILLRA
ANTHSSAKPCGQQSIHLGGVFELQPGASVFVNVTD
PSQVSHGTGFTSFGLLKLAAAHHHHHHV
CCD-hsCD70
(SEQ ID NO: 40)
METDTLLLWVLLLWVPGSTGDLKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISPLESLGWDVAE
LQLNHTGPQQDPRLYWQGGPALGRSFLHGPELDKG
QLRIHRDGIYMVHIQVTLAICSSTTASRHHPTTLA
VGICSPASRSISLLRLSFHQGCTIASQRLTPLARG
DTLCTNLTGTLLPSRNTDETFFGVQWVRPLAAAHH
HHHHV
CCD-hsCD137L
(SEQ ID NO: 41)
METDTLLLWVLLLWVPGSTGDLKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISRASPGSAASPR
LREGPELSPDDPAGLLDLRQGMFAQLVAQNVLLID
GPLSWYSDPGLAGVSLTGGLSYKEDTKELVVAKAG
VYYVFFQLELRRVVAGEGSGSVSLALHLQPLRSAA
GAAALALTVDLPPASSEARNSAFGFQGRLLHLSAG
QRLGVHLHTEARARHAWQLTQGATVLGLFRVTPEI
PAGLPSPRSEAAAHHHHHHV
CCD-Tumor necrosis factor receptor
superfamily member 4 ligand (hsOx40L)
(SEQ ID NO: 42)
METDTLLLWVLLLWVPGSTGDLKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISQSIKVQFTEYK
KEKGFILTSQKEDEIMKVQNNSVIINCDGFYLISL
KGYFSQEVNISLHYQKDEEPLFQLKKVRSVNSLMV
ASLTYKDKVYLNVTTDNTSLDDFHVNGGELILIHQ
NPGEFCVLAAAHHHHHHV
Cluster of Differentiation 276
(hsB7H3, or CD276)
(SEQ ID NO: 43)
MLRRRGSPGMGVHVGAALGALWFCLTGALEVQVPE
DPVVALVGTDATLCCSFSPEPGFSLAQLNLIWQLT
DTKQLVHSFAEGQDQGSAYANRTALFPDLLAQGNA
SLRLQRVRVADEGSFTCFVSIRDFGSAAVSLQVAA
PYSKPSMTLEPNKDLRPGDTVTITCSSYRGYPEAE
VFWQDGQGVPLTGNVTTSQMANEQGLFDVHSVLRV
VLGANGTYSCLVRNPVLQQDAHGSVTITGLKEFMG
YQGPPGPPGPPGIPGNHGNNGNNGATGHEGAKGEK
GDKGDLGPRGERGQHGPKGEKGYPGIPGYCGPPGP
PGPPGIPGNHGNNGNNGATGHEGAKGEKGDKGDLG
PRGERGQHGPKGEKGYPGIPDYKDDDDKLQISSTV
AAAHHHHHHV
V-set domain-containing T-cell
activation inhibitor 1
(hsB7H4, or VTCN1)
(SEQ ID NO: 44)
MASLGQILFWSIISIIIILAGAIALIIGFGISGRH
SITVTTVASAGNIGEDGILSCTFEPDIKLSDIVIQ
WLKEGVLGLVHEFKEGKDELSEQDEMFRGRTAVFA
DQVIVGNASLRLKNVQLTDAGTYKCYIITSKGKGN
ANLEYKTGAFSMPEVNVDYNASSETLRCEAPRWFP
QPTVVWASQVDQGANFSEVSNTSFELNSENVTMKV
VSVLYNVTINNTYSCMIENDIAKATGDIKVTESEI
KRRSHLKEFMGYQGPPGPPGPPGIPGNHGNNGNNG
ATGHEGAKGEKGDKGDLGPRGERGQHGPKGEKGYP
GIPGYCGPPGPPGPPGIPGNHGNNGNNGATGHEGA
KGEKGDKGDLGPRGERGQHGPKGEKGYPGIPDYKD
DDDKLQISSTVAAAHHHHHHV
hsBTLA
(SEQ ID NO: 45)
MKTLPAMLGTGKLFWVFFLIPYLDIWNIHGKESCD
VQLYIKRQSEHSILAGDPFELECPVKYCANRPHVT
WCKLNGTTCVKLEDRQTSWKEEKNISFFILHFEPV
LPNDNGSYRCSANFQSNLIESHSTTLYVTDVKSAS
ERPSKDEMLKEFMGYQGPPGPPGPPGIPGNHGNNG
NNGATGHEGAKGEKGDKGDLGPRGERGQHGPKGEK
GYPGIPGYCGPPGPPGPPGIPGNHGNNGNNGATGH
EGAKGEKGDKGDLGPRGERGQHGPKGEKGYPGIPD
YKDDDDKLQISSTVAAAHHHHHHV
hsCD27
(SEQ ID NO: 46)
MARPHPWWLCVLGTLVGLSATPAPKSCPERHYWAQ
GKLCCQMCEPGTFLVKDCDQHRKAAQCDPCIPGVS
FSPDHHTRPHCESCRHCNSGLLVRNCTITANAECA
CRNGWQCRDKECTECDPLPNPSLTARSSQALSPHP
QPTHLPYVSEMLEARTAGHMQTLADFRQLPARTLS
THWPPQRLKEFMGYQGPPGPPGPPGIPGNHGNNGN
NGATGHEGAKGEKGDKGDLGPRGERGQHGPKGEKG
YPGIPGYCGPPGPPGPPGIPGNHGNNGNNGATGHE
GAKGEKGDKGDLGPRGERGQHGPKGEKGYPGIPDY
KDDDDKLQISSTVAAAHHHHHHV
hsCD28
(SEQ ID NO: 47)
MLRLLLALNLFPSIQVTGNKILVKQSPMLVAYDNA
VNLSCKYSYNLFSREFRASLHKGLDSAVEVCVVYG
NYSQQLQVYSKTGFNCDGKLGNESVTFYLQNLYVN
QTDIYFCKIEVMYPPPYLDNEKSNGTIIHVKGKHL
CPSLKEFMGYQGPPGPPGPPGIPGNHGNNGNNGAT
GHEGAKGEKGDKGDLGPRGERGQHGPKGEKGYPGI
PGYCGPPGPPGPPGIPGNHGNNGNNGATGHEGAKG
EKGDKGDLGPRGERGQHGPKGEKGYPGIPDYKDDD
DKLQISSTVAAAHHHHHHV
hsCD40
(SEQ ID NO: 48)
MVRLPLQCVLWGCLLTAVHPEPPTACREKQYLINS
QCCSLCQPGQKLVSDCTEFTETECLPCGESEFLDT
WNRETHCHQHKYCDPNLGLRVQQKGTSETDTICTC
EEGWHCTSEACESCVLHRSCSPGFGVKQIATGVSD
TICEPCPVGFFSNVSSAFEKCHPWTSCETKDLVVQ
QAGTNKTDVLKEFMGYQGPPGPPGPPGIPGNHGNN
GNNGATGHEGAKGEKGDKGDLGPRGERGQHGPKGE
KGYPGIPGYCGPPGPPGPPGIPGNHGNNGNNGATG
HEGAKGEKGDKGDLGPRGERGQHGPKGEKGYPGIP
DYKDDDDKLQISSTVAAAHHHHHHV
hsCD80
(SEQ ID NO: 50)
MGHTRRQGTSPSKCPYLNFFQLLVLAGLSHFCSGV
IHVTKEVKEVATLSCGHNVSVEELAQTRIYWQKEK
KMVLTMMSGDMNIWPEYKNRTIFDITNNLSIVILA
LRPSDEGTYECVVLKYEKDAFKREHLAEVTLSVKA
DFPTPSISDFEIPTSNIRRIICSTSGGFPEPHLSW
LENGEELNAINTTVSQDPETELYAVSSKLDFNMTT
NHSFMCLIKYGHLRVNQTFNWNTLKEFMGYQGPPG
PPGPPGIPGNHGNNGNNGATGHEGAKGEKGDKGDL
GPRGERGQHGPKGEKGYPGIPGYCGPPGPPGPPGI
PGNHGNNGNNGATGHEGAKGEKGDKGDLGPRGERG
QHGPKGEKGYPGIPDYKDDDDKLQISSTVAAAHHH
HHHV
hsCD86
(SEQ ID NO: 51)
MDPQCTMGLSNILFVMAFLLSGAAPLKIQAYFNET
ADLPCQFANSQNQSLSELVVFWQDQENLVLNEVYL
GKEKFDSVHSKYMGRTSFDSDSWTLRLHNLQIKDK
GLYQCIIHHKKPTGMIRIHQMNSELSVLANFSQPE
IVPISNITENVYINLTCSSIHGYPEPKKMSVLLRT
KNSTIEYDGIMQKSQDNVTELYDVSISLSVSFPDV
TSNMTIFCILETDKTRLLSSPFSIELEDLKEFMGY
QGPPGPPGPPGIPGNHGNNGNNGATGHEGAKGEKG
DKGDLGPRGERGQHGPKGEKGYPGIPGYCGPPGPP
GPPGIPGNHGNNGNNGATGHEGAKGEKGDKGDLGP
RGERGQHGPKGEKGYPGIPDYKDDDDKLQISSTVA
AAHHHHHHV
hsCD137
(SEQ ID NO: 52)
MGNSCYNIVATLLLVLNFERTRSLQDPCSNCPAGT
FCDNNRNQICSPCPPNSFSSAGGQRTCDICRQCKG
VFRTRKECSSTSNAECDCTPGFHCLGAGCSMCEQD
CKQGQELTKKGCKDCCFGTFNDQKRGICRPWTNCS
LDGKSVLVNGTKERDVVCGPSPADLSPGASSVTPP
APLKEFMGYQGPPGPPGPPGIPGNHGNNGNNGATG
HEGAKGEKGDKGDLGPRGERGQHGPKGEKGYPGIP
GYCGPPGPPGPPGIPGNHGNNGNNGATGHEGAKGE
KGDKGDLGPRGERGQHGPKGEKGYPGIPDYKDDDD
KLQISSTVAAAHHHHH
HV
hsCTLA4
(SEQ ID NO: 53)
MACLGFQRHKAQLNLATRTWPCTLLFFLLFIPVFC
KAMHVAQPAVVLASSRGIASFVCEYASPGKATEVR
VTVLRQADSQVTEVCAATYMMGNELTFLDDSICTG
TSSGNQVNLTIQGLRAMDTGLYICKVELMYPPPYY
LGIGNGTQIYVLKEFMGYQGPPGPPGPPGIPGNHG
NNGNNGATGHEGAKGEKGDKGDLGPRGERGQHGPK
GEKGYPGIPGYCGPPGPPGPPGIPGNHGNNGNNGA
TGHEGAKGEKGDKGDLGPRGERGQHGPKGEKGYPG
IPDYKDDDDKLQISSTVAAAHHHHHHV
hsHAVCR2
(SEQ ID NO: 54)
MFSHLPFDCVLLLLLLLLTRSSEVEYRAEVGQNAY
LPCFYTPAAPGNLVPVCWGKGACPVFECGNVVLRT
DERDVNYWTSRYWLNGDFRKGDVSLTIENVTLADS
GIYCCRIQIPGIMNDEKFNLKLVIKPAKVTPAPTL
QRDFTAAFPRMLTTRGHGPAETQTLGSLPDINLTQ
ISTLANELRDSRLANDLRLKEFMGYQGPPGPPGPP
GIPGNHGNNGNNGATGHEGAKGEKGDKGDLGPRGE
RGQHGPKGEKGYPGIPGYCGPPGPPGPPGIPGNHG
NNGNNGATGHEGAKGEKGDKGDLGPRGERGQHGPK
GEKGYPGIPDYKDDDDKLQISSTVAAAHHHHHHV
hsHVEM
(SEQ ID NO: 55)
MEPPGDWGPPPWRSTPRTDVLRLVLYLTFLGAPCY
APALPSCKEDEYPVGSECCPKCSPGYRVKEACGEL
TGTVCEPCPPGTYIAHLNGLSKCLQCQMCDPAMGL
RASRNCSRTENAVCGCSPGHFCIVQDGDHCAACRA
YATSSPGQRVQKGGTESQDTLCQNCPPGTFSPNGT
LEECQHQTKCSWLVTKAGLKEFMGYQGPPGPPGPP
GIPGNHGNNGNNGATGHEGAKGEKGDKGDLGPRGE
RGQHGPKGEKGYPGIPGYCGPPGPPGPPGIPGNHG
NNGNNGATGHEGAKGEKGDKGDLGPRGERGQHGPK
GEKGYPGIPDYKDDDDKLQISSTVAAAHHHHHHV
hsIcos
(SEQ ID NO: 56)
MRLGSPGLLFLLFSSLRADTQEKEVRAMVGSDVEL
SCACPEGSRFDLNDVYVYWQTSESKTVVTYHIPQN
SSLENVDSRYRNRALMSPAGMLRGDFSLRLFNVTP
QDEQKFHCLVLSQSLGFQEVLSVEVTLHVAANFSV
PVVSAPHSPSQDELTFTCTSINGYPRPNVYWINKT
DNSLLDQALQNDTVFLNMRGLYDVVSVLRIARTPS
VNIGCCIENVLLQQNLTVGSQTGNDIGERDKITEN
PVLKEFMGYQGPPGPPGPPGIPGNHGNNGNNGATG
HEGAKGEKGDKGDLGPRGERGQHGPKGEKGYPGIP
GYCGPPGPPGPPGIPGNHGNNGNNGATGHEGAKGE
KGDKGDLGPRGERGQHGPKGEKGYPGIPDYKDDDD
KLQISSTVAAAHHHHHHV
hsLag3
(SEQ ID NO: 57)
MWEAQFLGLLFLQPLWVAPVKPLQPGAEVPVVWAQ
EGAPAQLPCSPTIPLQDLSLLRRAGVTWQHQPDSG
PPAAAPGHPLAPGPHPAAPSSWGPRPRRYTVLSVG
PGGLRSGRLPLQPRVQLDERGRQRGDFSLWLRPAR
RADAGEYRAAVHLRDRALSCRLRLRLGQASMTASP
PGSLRASDWVILNCSFSRPDRPASVHWFRNRGQGR
VPVRESPHHHLAESFLFLPQVSPMDSGPWGCILTY
RDGFNVSIMYNLTVLGLEPPTPLTVYAGAGSRVGL
PCRLPAGVGTRSFLTAKWTPPGGGPDLLVTGDNGD
FTLRLEDVSQAQAGTYTCHIHLQEQQLNATVTLAI
ITVTPKSFGSPGSLGKLLCEVTPVSGQERFVWSSL
DTPSQRSFSGPWLEAQEAQLLSQPWQCQLYQGERL
LGAAVYFTELSSPGAQRSGRALKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISSTVAAAHHHHH
HV
hsOx40
(SEQ ID NO: 58)
MCVGARRLGRGPCAALLLLGLGLSTVTGLHCVGDT
YPSNDRCCHECRPGNGMVSRCSRSQNTVCRPCGPG
FYNDVVSSKPCKPCTWCNLRSGSERKQLCTATQDT
VCRCRAGTQPLDSYKPGVDCAPCPPGHFSPGDNQA
CKPWTNCTLAGKHTLQPASNSSDAICEDRDPPATQ
PQETQGPPARPITVQPTEAWPRTSQGPSTRLKEFM
GYQGPPGPPGPPGIPGNHGNNGNNGATGHEGAKGE
KGDKGDLGPRGERGQHGPKGEKGYPGIPGYCGPPG
PPGPPGIPGNHGNNGNNGATGHEGAKGEKGDKGDL
GPRGERGQHGPKGEKGYPGIPDYKDDDDKLQISST
VAAAHHHHHHV
hsPD1
(SEQ ID NO: 59)
MQIPQAPWPVVWAVLQLGWRPGWFLDSPDRPWNPP
TFSPALLVVTEGDNATFTCSFSNTSESFVLNWYRM
SPSNQTDKLAAFPEDRSQPGQDCRFRVTQLPNGRD
FHMSVVRARRNDSGTYLCGAISLAPKAQIKESLRA
ELRVTERRAEVPTAHPSPSPRLKEFMGYQGPPGPP
GPPGIPGNHGNNGNNGATGHEGAKGEKGDKGDLGP
RGERGQHGPKGEKGYPGIPGYCGPPGPPGPPGIPG
NHGNNGNNGATGHEGAKGEKGDKGDLGPRGERGQH
GPKGEKGYPGIPDYKDDDDKLQISSTVAAAHHHHH
HV
hsPD-L1
(SEQ ID NO: 60)
MRIFAVFIFMTYWHLLNAFTVTVPKDLYVVEYGSN
MTIECKFPVEKQLDLAALIVYWEMEDKNIIQFVHG
EEDLKVQHSSYRQRARLLKDQLSLGNAALQITDVK
LQDAGVYRCMISYGGADYKRITVKVNAPYNKINQR
ILVVDPVTSEHELTCQAEGYPKAEVIWTSSDHQVL
SGKTTTTNSKREEKLFNVTSTLRINTTTNEIFYCT
FRRLDPEENHTAELVIPELLKEFMGYQGPPGPPGP
PGIPGNHGNNGNNGATGHEGAKGEKGDKGDLGPRG
ERGQHGPKGEKGYPGIPGYCGPPGPPGPPGIPGNH
GNNGNNGATGHEGAKGEKGDKGDLGPRGERGQHGP
KGEKGYPGIPDYKDDDDKLQISSTVAAAHHHHHHV
hsPD-L2
(SEQ ID NO: 61)
MIFLLLMLSLELQLHQIAALFTVTVPKELYIIEHG
SNVTLECNFDTGSHVNLGAITASLQKVENDTSPHR
ERATLLEEQLPLGKASFHIPQVQVRDEGQYQCIII
YGVAWDYKYLTLKVKASYRKINTHILKVPETDEVE
LTCQATGYPLAEVSWPNVSVPANTSHSRTPEGLYQ
VTSVLRLKPPPGRNFSCVFWNTHVRELTLASIDLQ
SLKEFMGYQGPPGPPGPPGIPGNHGNNGNNGATGH
EGAKGEKGDKGDLGPRGERGQHGPKGEKGYPGIPG
YCGPPGPPGPPGIPGNHGNNGNNGATGHEGAKGEK
GDKGDLGPRGERGQHGPKGEKGYPGIPDYKDDDDK
LQISSTVAAAHHHHHHV
T cell immunoreceptor with Ig and
ITIM domains (hsTIGIT)
(SEQ ID NO: 62)
MRWCLLLIWAQGLRQAPLASGMMTGTTETTGNISA
EKGGSIILQCHLSSTTAQVTQVNWEQQDQLLAICN
ADLGWHISPSFKDRVAPGPGLGLTLQSLTVNDTGE
YFCIYHTYPDGTYTGRIFLEVLESSVALKEFMGYQ
GPPGPPGPPGIPGNHGNNGNNGATGHEGAKGEKGD
KGDLGPRGERGQHGPKGEKGYPGIPGYCGPPGPPG
PPGIPGNHGNNGNNGATGHEGAKGEKGDKGDLGPR
GERGQHGPKGEKGYPGIPDYKDDDDKLQISSTVAA
AHHHHHHV
Anti-CD3e single chain fragment,
merozoite surface protein (scFv-MSP)
(mouse)
(SEQ ID NO: 63)
MNSGLQLVFFVLTLKGIQGEGSHHHHHHVQLVESG
GGLVQPGKSLKLSCEASGFTFSGYGMHWVRQAPGR
GLESVAYITSSSIMKYADAVKGRFTVSRDNAKNLL
FLQMNILKSEDTAMYYCARFDWDKNYWGQGTMVTV
SSGGGGSGGGGSGGGGSIQMTQSPSSLPASLGDRV
TINCQASQDISNYLNWYQQKPGKAPKLLIYYTNKL
ADGVPSRFSGSGSGRDSSFTISSLESEDIGSYYCQ
QYYNYPWTFGPGTKLEIKRDIGGGGSGGGGSDYKD
DDDKDGIFCSSSNFLGISFLLILMLILYSFI
Anti-CD3e scFv-MSP (human)
(SEQ ID NO: 64)
MERHWIFLLLLSVTAGVHSQVGSHHHHHHSQVQLQ
QSGAELARPGASVKMSCKASGYTFTRYTMHWVKQR
PGQGLEWIGYINPSRGYTNYNQKFKDKATLTTDKS
SSTAYMQLSSLTSEDSAVYYCARYYDDHYCLDYWG
QGTTLTVSSGGGGSGGGGSGGGGSQIVLTQSPAIM
SASPGEKVTMTCSASSSVSYMNWYQQKSGTSPKRW
IYDTSKLASGVPAHFRGSGSGTSYSLTISGMEAED
AATYYCQQWSSNPFTFGSGTKLEINRLKEFGGGGS
GGGGSDYKDDDDKDGIFCSSSNFLGISFLLILMLI
LYSFI
HSV-TK-CCD
(SEQ ID NO: 105)
WTMASYPCHQHASAFDQAARSRGHSNRRTALRPRR
QQEATEVRLEQKMPTLLRVYIDGPHGMGKTTTTQL
LVALGSRDDIVYVPEPMTYWQVLGASETIANIYTT
QHRLDQGEISAGDAAVVMTSAQITMGMPYAVTDAV
LAPHIGGEAGSSHAPPPALTLIFDRHPIAALLCYP
AARYLMGSMTPQAVLAFVALIPPTLPGTNIVLGAL
PEDRHIDRLAKRQRPGERLDLAMLAAIRRVYGLLA
NTVRYLQGGGSWREDWGQLSGTAVPPQGAEPQSNA
GPRPHIGDTLFTLFRAPELLAPNGDLYNVFAWALD
VLAKRLRPMHVFILDYDQSPAGCRDALLQLTSGMV
QTHVTTPGSIPTICDLARTFAREMGEANLKEFGGG
GSGGGGSGGGGSMGYQGPPGPPGPPGIPGNHGNNG
NNGATGHEGAKGEKGDKGDLGPRGERGQHGPKGEK
GYPGIPGYCGPPGPPGPPGIPGNHGNNGNNGATGH
EGAKGEKGDKGDLGPRGERGQHGPKGEKGYPGIPD
YKDDDDKGGGGSGGGGSGGGGSLQISTRAAAHHHH
HHV
HSV-TK SR39-CCD
(SEQ ID NO: 106)
MASYPCHQHASAFDQAARSRGHSNRRTALRPRRQQ
EATEVRLEQKMPTLLRVYIDGPHGMGKTTTTQLLV
ALGSRDDIVYVPEPMTYWQVLGASETIANIYTTQH
RLDQGEISAGDAAVVMTSAQITMGMPYAVTDAVLA
PHIGGEAGSSHAPPPALTIFLDRHPIAFMLCYPAA
RYLMGSMTPQAVLAFVALIPPTLPGTNIVLGALPE
DRHIDRLAKRQRPGERLDLAMLAAIRRVYGLLANT
VRYLQGGGSWREDWGQLSGTAVPPQGAEPQSNAGP
RPHIGDTLFTLFRAPELLAPNGDLYNVFAWALDVL
AKRLRPMHVFILDYDQSPAGCRDALLQLTSGMVQT
HVTTPGSIPTICDLARTFAREMGEANLKEFGGGGS
GGGGSGGGGSMGYQGPPGPPGPPGIPGNHGNNGNN
GATGHEGAKGEKGDKGDLGPRGERGQHGPKGEKGY
PGIPGYCGPPGPPGPPGIPGNHGNNGNNGATGHEG
AKGEKGDKGDLGPRGERGQHGPKGEKGYPGIPDYK
DDDDKGGGGSGGGGSGGGGSLQISTRAAAHHHHHH
V
hGBA-CCD
(SEQ ID NO: 107)
MEFSSPSREECPKPLSRVSIMAGSLTGLLLLQAVS
WASGARPCIPKSFGYSSVVCVCNATYCDSFDPPTF
PALGTFSRYESTRSGRRMELSMGPIQANHTGTGLL
LTLQPEQKFQKVKGFGGAMTDAAALNILALSPPAQ
NLLLKSYFSEEGIGYNIIRVPMASCDFSIRTYTYA
DTPDDFQLHNFSLPEEDTKLKIPLIHRALQLAQRP
VSLLASPWTSPTWLKTNGAVNGKGSLKGQPGDIYH
QTWARYFVKFLDAYAEHKLQFWAVTAENEPSAGLL
SGYPFQCLGFTPEHQRDFIARDLGPTLANSTHHNV
RLLMLDDQRLLLPHWAKVVLTDPEAAKYVHGIAVH
WYLDFLAPAKATLGETHRLFPNTMLFASEACVGSK
FWEQSVRLGSWDRGMQYSHSIITNLLYHVVGWTDW
NLALNPEGGPNWVRNFVDSPIIVDITKDTFYKQPM
FYHLGHFSKFIPEGSQRVGLVASQKNDLDAVALMH
PDGSAVVVVLNRSSKDVPLTIKDPAVGFLETISPG
YSIHTYLWRRQLKEFMGYQGPPGPPGPPGIPGNHG
NNGNNGATGHEGAKGEKGDKGDLGPRGERGQHGPK
GEKGYPGIPGYCGPPGPPGPPGIPGNHGNNGNNGA
TGHEGAKGEKGDKGDLGPRGERGQHGPKGEKGYPG
IPDYKDDDDKLQISSTVAAAHHHHHHV
hGBA-CCD-Aβ9 scFv
(SEQ ID NO: 108)
MEFSSPSREECPKPLSRVSIMAGSLTGLLLLQAVS
WASGARPCIPKSFGYSSVVCVCNATYCDSFDPPTF
PALGTFSRYESTRSGRRMELSMGPIQANHTGTGLL
LTLQPEQKFQKVKGFGGAMTDAAALNILALSPPAQ
NLLLKSYFSEEGIGYNIIRVPMASCDFSIRTYTYA
DTPDDFQLHNFSLPEEDTKLKIPLIHRALQLAQRP
VSLLASPWTSPTWLKTNGAVNGKGSLKGQPGDIYH
QTWARYFVKFLDAYAEHKLQFWAVTAENEPSAGLL
SGYPFQCLGFTPEHQRDFIARDLGPTLANSTHHNV
RLLMLDDQRLLLPHWAKVVLTDPEAAKYVHGIAVH
WYLDFLAPAKATLGETHRLFPNTMLFASEACVGSK
FWEQSVRLGSWDRGMQYSHSIITNLLYHVVGWTDW
NLALNPEGGPNWVRNFVDSPIIVDITKDTFYKQPM
FYHLGHFSKFIPEGSQRVGLVASQKNDLDAVALMH
PDGSAVVVVLNRSSKDVPLTIKDPAVGFLETISPG
YSIHTYLWRRQLKEFMGYQGPPGPPGPPGIPGNHG
NNGNNGATGHEGAKGEKGDKGDLGPRGERGQHGPK
GEKGYPGIPGYCGPPGPPGPPGIPGNHGNNGNNGA
TGHEGAKGEKGDKGDLGPRGERGQHGPKGEKGYPG
IPDYKDDDDKLQISGGGGSGGGGSQVTLKESGPGI
LQPSQTLSLTCSFSGFSLNTFGMGVSWIRQPSGKG
LEWLAHIFWDDDKHYNPSLKSRLTISKDTSNNQVF
LKITTVDTADTATYYCVRYGFDGFPYWGQGTLVTV
SAGGGGSGGGGSGGGGSDVVMTQTPLSLPVSLGDQ
ASISCRSNQSLVHSNGNTYLHWYLQKPGQSPKLLI
YKVSNRFSGVPDRFSGSGSGTDFTLKISRVEAEDL
GFYFCSQSTRVPWTFGGGTKLELKRAAAHHHHHHV

Exemplary fusion proteins comprising heterologous peptides that comprise cell signaling modulators are described herein. Unless otherwise indicated in the fusion protein name, the CCD is positioned at the C-terminus of the protein. In various embodiments, the disclosed fusion proteins comprise one or more heterologous peptides comprising an amino acid sequence that is at least 85%, at least 90%, at least 95%, at least 98%, at least 99%, or at least 99.5% identical to the amino acid sequences that follow. Any of the disclosed fusion proteins may differ relative to any of the following amino acid sequences by 1, 2, 3, 4, 5, 6, 7, 8, 9, 10, 10-15, 15-20, 20-30, 30-50, or more than 50 amino acids. These differences may comprise amino acids that have been inserted, deleted, or substituted relative to the sequence of any of SEQ ID NOs: 65-91. In some embodiments, the disclosed fusion proteins contain stretches of about 10, about 20, about 25, about 40, 50, about 75, about 100, about 125, about 150, about 175, or about 180 amino acids in common with the sequence of any of SEQ ID NOs: 65-91.

Many of the sequences that follow (SEQ ID NOs: 65-91) are illustrated with poly(A) tails and polyhistidine tags, or other tags, at the C terminus. However, in some embodiments, and as can readily be visualized by one skilled in the art, the disclosed exemplary fusion proteins do not contain these tags.

(“s” designates soluble proteins; “hs” designates human sequences):

Interleukin-10 receptor (sIL10R)
(SEQ ID NO: 65)
MLPCLVVLLAALLSLRLGSDAHGTELPSPPSVWFEAEFFHHILHWTPIPNQ
SESTCYEVALLRYGIESWNSISNCSQTLSYDLTAVTLDLYHSNGYRARVRAVDGSRH
SNWTVTNTRFSVDEVTLTVGSVNLEIHNGFILGKIQLPRPKMAPANDTYESIFSHFRE
YEIAIRKVPGNFTFTHKKVKHENFSLLTSGEVGEFCVQVKPSVASRSNKGMWSKEEC
ISLLKEFMGYQGPPGPPGPPGIPGNHGNNGNNGATGHEGAKGEKGDKGDLGPRGER
GQHGPKGEKGYPGIPGYCGPPGPPGPPGIPGNHGNNGNNGATGHEGAKGEKGDKGD
LGPRGERGQHGPKGEKGYPGIPDYKDDDDKLQISSTVAAAHHHHHHV
Triggering receptor expressed on myeloid cells 1 (hsTREM1)
(SEQ ID NO: 66)
MRKTRLWGLLWMLFVSELRAATKLTEEKYELKEGQTLDVKCDYTLEKF
ASSQKAWQIIRDGEMPKTLACTERPSKNSHPVQVGRIILEDYHDHGLLRVRMVNLQV
EDSGLYQCVIYQPPKEPHMLFDRIRLVVTKGFSGTPGSNENSTQNVYKIPPTTTKALC
PLYTSPRKDPGHLVEFMGYQGPPGPPGPPGIPGNHGNNGNNGATGHEGAKGEKGDK
GDLGPRGERGQHGPKGEKGYPGIPGYCGPPGPPGPPGIPGNHGNNGNNGATGHEGA
KGEKGDKGDLGPRGERGQHGPKGEKGYPGIPDYKDDDDKLQISSTVAAARGRRKR
TREM-like transcript 1 (hsTREML1)
(SEQ ID NO: 67)
MGLTLLLLLLLGLEGQGIVGSLPEVLQAPVGSSILVQCHYRLQDVKAQKV
WCRFLPEGCQPLVSSAVDRRAPAGRRTFLTDLGGGLLQVEMVTLQEEDAGEYGCM
VDGARGPQILHRVSLNILPPEEEEETHKIGSLAENLKEFMGYQGPPGPPGPPGIPGNHG
NNGNNGATGHEGAKGEKGDKGDLGPRGERGQHGPKGEKGYPGIPGYCGPPGPPGPP
GIPGNHGNNGNNGATGHEGAKGEKGDKGDLGPRGERGQHGPKGEKGYPGIPDYKD
DDDKLQISSTVAAAHHHHHHV
Toll-like receptor 5 (sTLR5)
(SEQ ID NO: 68)
MGDHLDLLLGVVLMAGPVFGIPSCSFDGRIAFYRFCNLTQVPQVLNTTER
LLLSFNYIRTVTASSFPFLEQLQLLELGSQYTPLTIDKEAFRNLPNLRILDLGSSKIYFL
HPDAFQGLFHLFELRLYFCGLSDAVLKDGYFRNLKALTRLDLSKNQIRSLYLHPSFG
KLNSLKSIDFSSNQIFLVCEHELEPLQGKTLSFFSLAANSLYSRVSVDWGKCMNPFRN
MVLEILDVSGNGWTVDITGNFSNAISKSQAFSLILAHHIMGAGFGFHNIKDPDQNTFA
GLARSSVRHLDLSHGFVFSLNSRVFETLKDLKVLNLAYNKINKIADEAFYGLDNLQV
LNLSYNLLGELYSSNFYGLPKVAYIDLQKNHIAIIQDQTFKFLEKLRTLDLRDNALTTI
HFIPSIPDIFLSGNKLVTLPKINLTANLIHLSENRLENLDILYFLLRVPHLQILILNQNRFS
SCSGDQTPSENPSLEQLFLGENMLQLAWETELCWDVFEGLSHLQVLYLNHNYLNSLP
PGVFSHLTALRGLSLNSNRLTVLSHNDLPANLEILDISRNQLLAPNPDVFVSLSVLDIT
HNKFICECELSTFINWLNHTNVTIAGPPADIYCVYPDSFSGVSLFSLSTEGCDEEEVLK
GSMGYQGPPGPPGPPGIPGNHGNNGNNGATGHEGAKGEKGDKGDLGPRGERGQHG
PKGEKGYPGIPGYCGPPGPPGPPGIPGNHGNNGNNGATGHEGAKGEKGDKGDLGPR
GERGQHGPKGEKGYPGIPDYKDDDDKLQISSTVAAARV
Hedgehog interacting protein (hHHIP)
(SEQ ID NO: 69)
MLKMLSFKLLLLAVALGFFEGDAKFGERNEGSGARRRRCLNGNPPKRLK
RRDRRMMSQLELLSGGEMLCGGFYPRLSCCLRSDSPGLGRLENKIFSVTNNTECGKL
LEEIKCALCSPHSQSLFHSPEREVLERDLVLPLLCKDYCKEFFYTCRGHIPGFLQTTAD
EFCFYYARKDGGLCFPDFPRKQVRGPASNYLDQMEEYDKVEEISRKHKHNCFCIQEV
VSGLRQPVGALHSGDGSQRLFILEKEGYVKILTPEGEIFKEPYLDIHKLVQSGIKGGDE
RGLLSLAFHPNYKKNGKLYVSYTTNQERWAIGPHDHILRVVEYTVSRKNPHQVDLR
TARVFLEVAELHRKHLGGQLLFGPDGFLYIILGDGMITLDDMEEMDGLSDFTGSVLR
LDVDTDMCNVPYSIPRSNPHFNSTNQPPEVFAHGLHDPGRCAVDRHPTDININLTILC
SDSNGKNRSSARILQIIKGKDYESEPSLLEFKPFSNGPLVGGFVYRGCQSERLYGSYVF
GDRNGNFLTLQQSPVTKQWQEKPLCLGTSGSCRGYFSGHILGFGEDELGEVYILSSSK
SMTQTHNGKLYKIVDPKRPLMPEECRATVQPLKEFMGYQGPPGPPGPPGIPGNHGNN
GNNGATGHEGAKGEKGDKGDLGPRGERGQHGPKGEKGYPGIPGYCGPPGPPGPPGI
PGNHGNNGNNGATGHEGAKGEKGDKGDLGPRGERGQHGPKGEKGYPGIPDYKDD
DDKLQISSTVAAAHHHHHHV
Vascular endothelial growth factor receptor 1 (hsVEGFR1)
(SEQ ID NO: 70)
MVSYWDTGVLLCALLSCLLLTGSSSGSKLKDPELSLKGTQHIMQAGQTLH
LQCRGEAAHKWSLPEMVSKESERLSITKSACGRNGKQFCSTLTLNTAQANHTGFYSC
KYLAVPTSKKKETESAIYIFISDTGRPFVEMYSEIPEIIHMTEGRELVIPCRVTSPNITVT
LKKFPLDTLIPDGKRIIWDSRKGFIISNATYKEIGLLTCEATVNGHLYKTNYLTHRQTN
TIIDVQISTPRPVKLLRGHTLVLNCTATTPLNTRVQMTWSYPDEKNKRASVRRRIDQS
NSHANIFYSVLTIDKMQNKDKGLYTCRVRSGPSFKSVNTSVHIYDKAFITVKHRKQQ
VLETVAGKRSYRLSMKVKAFPSPEVVWLKDGLPATEKSARYLTRGYSLIIKDVTEED
AGNYTILLSIKQSNVFKNLTATLIVNVKPQIYEKAVSSFPDPALYPLGSRQILTCTAYGI
PQPTIKWFWHPCNHNHSEARCDFCSNNEESFILDADSNMGNRIESITQRMAIIEGKNK
MASTLVVADSRISGIYICIASNKVGTVGRNISFYITDVPNGFHVNLEKMPTEGEDLKLS
CTVNKFLYRDVTWILLRTVNNRTMHYSISKQKMAITKEHSITLNLTIMNVSLQDSGT
YACRARNVYTGEEILQKKEITIRDQEAPYLLRNLSDHTVAISSSTTLDCHANGVPEPQI
TWFKNNHKIQQEPGIILGPGSSTLFIERVTEEDEGVYHCKATNQKGSVESSAYLTVQL
KEFMGYQGPPGPPGPPGIPGNHGNNGNNGATGHEGAKGEKGDKGDLGPRGERGQH
GPKGEKGYPGIPGYCGPPGPPGPPGIPGNHGNNGNNGATGHEGAKGEKGDKGDLGP
RGERGQHGPKGEKGYPGIPDYKDDDDKLQISSTVAAAHHHHHHV
Vascular endothelial growth factor receptor 2 (hsVEGFR2)
(SEQ ID NO: 71)
MQSKVLLAVALWLCVETRAASVGLPSVSLDLPRLSIQKDILTIKANTTLQI
TCRGQRDLDWLWPNNQSGSEQRVEVTECSDGLFCKTLTIPKVIGNDTGAYKCFYRE
TDLASVIYVYVQDYRSPFIASVSDQHGVVYITENKNKTVVIPCLGSISNLNVSLCARY
PEKRFVPDGNRISWDSKKGFTIPSYMISYAGMVFCEAKINDESYQSIMYIVVVVGYRI
YDVVLSPSHGIELSVGEKLVLNCTARTELNVGIDFNWEYPSSKHQHKKLVNRDLKTQ
SGSEMKKFLSTLTIDGITRSDQGLYTCAASSGLMTKKNSTFVRVHEKPFVAFGSGME
SLVEATVGERVRIPAKYLGYPPPEIKWYKNGIPLESNHTIKAGHVLTIMEVSERDTGN
YTVILTNPISKEKQSHVVSLVVYVPPQIGEKSLISPVDSYQYGTTQTLTCTVYAIPPPH
HIHWYWQLEEECANEPSQAVSVTNPYPCEEWRSVEDFQGGNKIEVNKNQFALIEGK
NKTVSTLVIQAANVSALYKCEAVNKVGRGERVISFHVTRGPEITLQPDMQPTEQESV
SLWCTADRSTFENLTWYKLGPQPLPIHVGELPTPVCKNLDTLWKLNATMFSNSTNDI
LIMELKNASLQDQGDYVCLAQDRKTKKRHCVVRQLTVLERVAPTITGNLENQTTSIG
ESIEVSCTASGNPPPQIMWFKDNETLVEDSGIVLKDGNRNLTIRRVRKEDEGLYTCQA
CSVLGCAKVEAFFIIELKEFMGYQGPPGPPGPPGIPGNHGNNGNNGATGHEGAKGEK
GDKGDLGPRGERGQHGPKGEKGYPGIPGYCGPPGPPGPPGIPGNHGNNGNNGATGH
EGAKGEKGDKGDLGPRGERGQHGPKGEKGYPGIPDYKDDDDKLQISSTVAAAHHH
HHHV
Fibroblast growth factor receptor 1 (hsFGFR1)
(SEQ ID NO: 72)
MWSWKCLLFWAVLVTATLCTARPSPTLPEQAQPWGAPVEVESFLVHPGD
LLQLRCRLRDDVQSINWLRDGVQLAESNRTRITGEEVEVQDSVPADSGLYACVTSSP
SGSDTTYFSVNVSDALPSSEDDDDDDDSSSEEKETDNTKPNPVAPYWTSPEKMEKKL
HAVPAAKTVKFKCPSSGTPNPTLRWLKNGKEFKPDHRIGGYKVRYATGSIIMDSVVP
SDKGNYTCIVENEYGSINHTYQLDVVERSPHRPILQAGLPANKTVALGSNVEFMCKV
YSDPQPHIQWLKHIEVNGSKIGPDNLPYVQILKTAGVNTTDKEMEVLHLRNVSFEDA
GEYTCLAGNSIGLSHHSAWLTVLEALEERPAVMLKEFMGYQGPPGPPGPPGIPGNHG
NNGNNGATGHEGAKGEKGDKGDLGPRGERGQHGPKGEKGYPGIPGYCGPPGPPGPP
GIPGNHGNNGNNGATGHEGAKGEKGDKGDLGPRGERGQHGPKGEKGYPGIPDYKD
DDDKLQISSTVAAAHHHHHHV
Secreted frizzled-related protein 1 (hSFRP1)
(SEQ ID NO: 73)
MGIGRSEGGRRGAALGVLLALGAALLAVGSASEYDYVSFQSDIGPYQSGR
FYTKPPQCVDIPADLRLCHNVGYKKMVLPNLLEHETMAEVKQQASSWVPLLNKNC
HAGTQVFLCSLFAPVCLDRPIYPCRWLCEAVRDSCEPVMQFFGFYWPEMLKCDKFPE
GDVCIAMTPPNATEASKPQGTTVCPPCDNELKSEAIIEHLCASEFALRMKIKEVKKEN
GDKKIVPKKKKPLKLGPIKKKDLKKLVLYLKNGADCPCHQLDNLSHHFLIMGRKVK
SQYLLTAIHKWDKKNKEFKNFMKKMKNHECPTFQSVFKLKEFMGYQGPPGPPGPPG
IPGNHGNNGNNGATGHEGAKGEKGDKGDLGPRGERGQHGPKGEKGYPGIPGYCGP
PGPPGPPGIPGNHGNNGNNGATGHEGAKGEKGDKGDLGPRGERGQHGPKGEKGYP
GIPDYKDDDDKLQISSTVAAAHHHHHHV
Epidermal growth factor receptor (hsEGFR)
(SEQ ID NO: 74)
MRPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIPLENLQIIRG
NMYYENSYALAVLSNYDANKTGLKELPMRNLQEILHGAVRFSNNPALCNVESIQWR
DIVSSDFLSNMSMDFQNHLGSCQKCDPSCPNGSCWGAGEENCQKLTKIICAQQCSGR
CRGKSPSDCCHNQCAAGCTGPRESDCLVCRKFRDEATCKDTCPPLMLYNPTTYQMD
VNPEGKYSFGATCVKKCPRNYVVTDHGSCVRACGADSYEMEEDGVRKCKKCEGPC
RKVCNGIGIGEFKDSLSINATNIKHFKNCTSISGDLHILPVAFRGDSFTHTPPLDPQELD
ILKTVKEITGFLLIQAWPENRTDLHAFENLEIIRGRTKQHGQFSLAVVSLNITSLGLRSL
KEISDGDVIISGNKNLCYANTINWKKLFGTSGQKTKIISNRGENSCKATGQVCHALCS
PEGCWGPEPRDCVSCRNVSRGRECVDKCNLLEGEPREFVENSECIQCHPECLPQAMN
ITCTGRGPDNCIQCAHYIDGPHCVKTCPAGVMGENNTLVWKYADAGHVCHLCHPN
CTYGCTGPGLEGCLKEFMGYQGPPGPPGPPGIPGNHGNNGNNGATGHEGAKGEKGD
KGDLGPRGERGQHGPKGEKGYPGIPGYCGPPGPPGPPGIPGNHGNNGNNGATGHEG
AKGEKGDKGDLGPRGERGQHGPKGEKGYPGIPDYKDDDDKLQISSTVAAAHHHHH
HV
Triggering receptor expressed on myeloid cells 2 (hsTREM2)
(SEQ ID NO: 75)
MEPLRLLILLFVTELSGAHNTTVFQGVAGQSLQVSCPYDSMKHWGRRKA
WCRQLGEKGPCQRVVSTHNLWLLSFLRRWNGSTAIKDDTLGGTLTITLRNLQPHDA
GLYQCQSLHGSEADTLRKVLVEVLADPLDHRDAGDLWFPGESESFEDAHVEHSISRS
LLELKEFMGYQGPPGPPGPPGIPGNHGNNGNNGATGHEGAKGEKGDKGDLGPRGER
GQHGPKGEKGYPGIPGYCGPPGPPGPPGIPGNHGNNGNNGATGHEGAKGEKGDKGD
LGPRGERGQHGPKGEKGYPGIPDYKDDDDKLQISSTVAAAHHHHHHV
TREM-like transcript 2 protein (hsTREML2)
(SEQ ID NO: 76)
MAPAFLLLLLLWPQGCVSGPSADSVYTKVRLLEGETLSVQCSYKGYKNR
VEGKVWCKIRKKKCEPGFARVWVKGPRYLLQDDAQAKVVNITMVALKLQDSGRY
WCMRNTSGILYPLMGFQLDVSPAPQSERNIPFTHLDNILKSGTVTTGQAPTSGPDAPF
TTGVMVFTPGLITLPRLLASTRPASKTGYSFTATSTTSQGPRRTMGSQTVTASPSNAR
DSSAGPESISTKSGDLSTRSPTTGLCLTSRSLLNRLPSMPLKEFIEGRMGYQGPPGPPGP
PGIPGNHGNNGNNGATGHEGAKGEKGDKGDLGPRGERGQHGPKGEKGYPGIPGYC
GPPGPPGPPGIPGNHGNNGNNGATGHEGAKGEKGDKGDLGPRGERGQHGPKGEKG
YPGIPDYKDDDDKLQISSTVAAAHHHHHHV
Notch homolog 1 (hsNOTCH1)
(SEQ ID NO: 77)
MPPLLAPLLCLALLPALAARGPRCSQPGETCLNGGKCEAANGTEACVCGG
AFVGPRCQDPNPCLSTPCKNAGTCHVVDRRGVADYACSCALGFSGPLCLTPLDNAC
LTNPCRNGGTCDLLTLTEYKCRCPPGWSGKSCQQADPCASNPCANGGQCLPFEASYI
CHCPPSFHGPTCRQDVNECGQKPRLCRHGGTCHNEVGSYRCVCRATHTGPNCERPY
VPCSPSPCQNGGTCRPTGDVTHECACLPGFTGQNCEENIDDCPGNNCKNGGACVDG
VNTYNCPCPPEWTGQYCTEDVDECQLMPNACQNGGTCHNTHGGYNCVCVNGWTG
EDCSENIDDCASAACFHGATCHDRVASFYCECPHGRTGLLCHLNDACISNPCNEGSN
CDTNPVNGKAICTCPSGYTGPACSQDVDECSLGANPCEHAGKCINTLGSFECQCLQG
YTGPRCEIDVNECVSNPCQNDATCLDQIGEFQCMCMPGYEGVHCEVNTDECASSPCL
HNGRCLDKINEFQCECPTGFTGHLCQEFMGYQGPPGPPGPPGIPGNHGNNGNNGATG
HEGAKGEKGDKGDLGPRGERGQHGPKGEKGYPGIPGYCGPPGPPGPPGIPGNHGNN
GNNGATGHEGAKGEKGDKGDLGPRGERGQHGPKGEKGYPGIPDYKDDDDKLQISS
TVAAAHHHHHHV
Colony stimulating factor 1 receptor (hsCSF1R)
(SEQ ID NO: 78)
MGPGVLLLLLVATAWHGQGIPVIEPSVPELVVKPGATVTLRCVGNGSVE
WDGPPSPHWTLYSDGSSSILSTNNATFQNTGTYRCTEPGDPLGGSAAIHLYVKDPAR
PWNVLAQEVVVFEDQDALLPCLLTDPVLEAGVSLVRVRGRPLMRHTNYSFSPWHGF
TIHRAKFIQSQDYQCSALMGGRKVMSISIRLKVQKVIPGPPALTLVPAELVRIRGEAA
QIVCSASSVDVNFDVFLQHNNTKLAIHQQSDFHNNRYQKVLTLNLDQVDFQHAGNY
SCVASNVQGKHSTSMFFRVVESAYLNLSSEQNLIQEVTVGEGLNLKVMVEAYPGLQ
GFNWTYLGPFSDHQPEPKLANVTTKDTYRHTFTLSLPRLKPSEAGRYSFLARNPGGW
RALTFELTLRYPPEVSVIWTFINGSGTLLCAASGYPQPNVTWLQCSGHTDRCDEAQV
LQVWDDPYPEVLSQEPFHKVTVQSLLTVETLEHNQTYECRAHNSVGSGSWAFIPISA
GAHTHLKEFMGYQGPPGPPGPPGIPGNHGNNGNNGATGHEGAKGEKGDKGDLGPR
GERGQHGPKGEKGYPGIPGYCGPPGPPGPPGIPGNHGNNGNNGATGHEGAKGEKGD
KGDLGPRGERGQHGPKGEKGYPGIPDYKDDDDKLQISSTVAAAHHHHHHV
CCD-TNF-related apoptosis-inducing ligand (hsTRAIL)
(SEQ ID NO: 79)
METDTLLLWVLLLWVPGSTGDLKEFMGYQGPPGPPGPPGIPGNHGNNGN
NGATGHEGAKGEKGDKGDLGPRGERGQHGPKGEKGYPGIPGYCGPPGPPGPPGIPG
NHGNNGNNGATGHEGAKGEKGDKGDLGPRGERGQHGPKGEKGYPGIPDYKDDDD
KLQISSKSGIACFLKEDDSYWDPNDEESMNSPCWQVKWQLRQLVRKMILRTSEETIS
TVQEKQQNISPLVRERGPQRVAAHITGTRGRSNTLSSPNSKNEKALGRKINSWESSRS
GHSFLSNLHLRNGELVIHEKGFYYIYSQTYFRFQEEIKENTKNDKQMVQYIYKYTSYP
DPILLMKSARNSCWSKDAEYGLYSIYQGGIFELKENDRIFVSVTNEHLIDMDHEASFF
GAFLVGAAAHHHHHHV
hsWIF1
(SEQ ID NO: 80)
MARRSAFPAAALWLWSILLCLLALRAEAGPPQEESLYLWIDAHQARVLIG
FEEDILIVSEGKMAPFTHDFRKAQQRMPAIPVNIHSMNFTWQAAGQAEYFYEFLSLRS
LDKGIMADPTVNVPLLGTVPHKASVVQVGFPCLGKQDGVAAFEVDVIVMNSEGNTI
LKTPQNAIFFKTCQQAECPGGCRNGGFCNERRICECPDGFHGPHCEKALCTPRCMNG
GLCVTPGFCICPPGFYGVNCDKANCSTTCFNGGTCFYPGKCICPPGLEGEQCEISKCP
QPCRNGGKCIGKSKCKCSKGYQGDLCSKPVCEPGCGAHGTCHEPNKCQCQEGWHG
RHCNKRYEASLIHALRPAGAQLRQHTPSLKKAEERRDPPESNYIWLKEFMGYQGPPG
PPGPPGIPGNHGNNGNNGATGHEGAKGEKGDKGDLGPRGERGQHGPKGEKGYPGIP
GYCGPPGPPGPPGIPGNHGNNGNNGATGHEGAKGEKGDKGDLGPRGERGQHGPKG
EKGYPGIPDYKDDDDKLQISSTVAAAHHHHHHV
hsDKK1
(SEQ ID NO: 81)
MMALGAAGATRVFVAMVAAALGGHPLLGVSATLNSVLNSNAIKNLPPPL
GGAAGHPGSAVSAAPGILYPGGNKYQTIDNYQPYPCAEDEECGTDEYCASPTRGGD
AGVQICLACRKRRKRCMRHAMCCPGNYCKNGICVSSDQNHFRGEIEETITESFGNDH
STLDGYSRRTTLSSKMYHTKGQEGSVCLRSSDCASGLCCARHFWSKICKPVLKEGQV
CTKHRRKGSHGLEIFQRCYCGEGLSCRIQKDHHQASNSSRLHTCQRHLKEFMGYQGP
PGPPGPPGIPGNHGNNGNNGATGHEGAKGEKGDKGDLGPRGERGQHGPKGEKGYP
GIPGYCGPPGPPGPPGIPGNHGNNGNNGATGHEGAKGEKGDKGDLGPRGERGQHGP
KGEKGYPGIPDYKDDDDKLQISSTVAAAHHHHHHV
hSFRP2
(SEQ ID NO: 82)
MLQGPGSLLLLFLASHCCLGSARGLFLFGQPDFSYKRSNCKPIPVNLQLCH
GIEYQNMRLPNLLGHETMKEVLEQAGAWIPLVMKQCHPDTKKFLCSLFAPVCLDDL
DETIQPCHSLCVQVKDRCAPVMSAFGFPWPDMLECDRFPQDNDLCIPLASSDHLLPA
TEEAPKVCEACKNKNDDDNDIMETLCKNDFALKIKVKEITYINRDTKIILETKSKTIY
KLNGVSERDLKKSVLWLKDSLQCTCEEMNDINAPYLVMGQKQGGELVITSVKRWQ
KGQREFKRISRSIRKLQCLKEFMGYQGPPGPPGPPGIPGNHGNNGNNGATGHEGAKG
EKGDKGDLGPRGERGQHGPKGEKGYPGIPGYCGPPGPPGPPGIPGNHGNNGNNGAT
GHEGAKGEKGDKGDLGPRGERGQHGPKGEKGYPGIPDYKDDDDKLQISSTVAAAH
HHHHHV
hsDLL3
(SEQ ID NO: 83)
MVSPRMSGLLSQTVILALIFLPQTRPAGVFELQIHSFGPGPGPGAPRSPCSA
RLPCRLFFRVCLKPGLSEEAAESPCALGAALSARGPVYTEQPGAPAPDLPLPDGLLQV
PFRDAWPGTFSFIIETWREELGDQIGGPAWSLLARVAGRRRLAAGGPWARDIQRAGA
WELRFSYRARCEPPAVGTACTRLCRPRSAPSRCGPGLRPCAPLEDECEAPPVCRAGCS
PEHGFCEQPGECRCLEGWTGPLCTVPVSTSSCLSPRGPSSATTGCLVPGPGPCDGNPC
ANGGSCSETPRSFECTCPRGFYGLRCEVSGVTCADGPCFNGGLCVGGADPDSAYICH
CPPGFQGSNCEKRVDRCSLQPCRNGGLCLDLGHALRCRCRAGFAGPRCEHDLDDCA
GRACANGGTCVEGGGAHRCSCALGFGGRDCRERADPCAARPCAHGGRCYAHFSGL
VCACAPGYMGARCLKEFMGYQGPPGPPGPPGIPGNHGNNGNNGATGHEGAKGEKG
DKGDLGPRGERGQHGPKGEKGYPGIPGYCGPPGPPGPPGIPGNHGNNGNNGATGHE
GAKGEKGDKGDLGPRGERGQHGPKGEKGYPGIPDYKDDDDKLQISSTVAAAHHHH
HHV
Neurogenic locus notch homolog 4 (hsNOTCH4)
(SEQ ID NO: 84)
MQPPSLLLLLLLLLLLCVSVVRPRGLLCGSFPEPCANGGTCLSLSLGQGTC
QCAPGFLGETCQFPDPCQNAQLCQNGGSCQALLPAPLGLPSSPSPLTPSFLCTCLPGFT
GERCQAKLEDPCPPSFCSKRGRCHIQASGRPQCSCMPGWTGEQCQLRDFCSANPCVN
GGVCLATYPQIQCHCPPGFEGHACERDVNECFQDPGPCPKGTSCHNTLGSFQCLCPV
GQEGPRCELRAGPCPPRGCSNGGTCQLMPEKDSTFHLCLCPPGFIGPDCEVNPDNCVS
HQCQNGGTCQDGLDTYTCLCPETWTGWDCSEDVDECETQGPPHCRNGGTCQNSAG
SFHCVCVSGWGGTSCEENLDDCIAATCAPGSTCIDRVGSFSCLCPPGRTGLLCHLED
MCLSQPCHGDAQCSTNPLTGSTLCLCQPGYSGPTCHQDLDECLMAQQGPSPCEHGG
SCLNTPGSFNCLCPPGYTGSRCEADHNECLSQPCHPGSTCLDLLATFHCLCPPGLEGQ
LCELKEFMGYQGPPGPPGPPGIPGNHGNNGNNGATGHEGAKGEKGDKGDLGPRGER
GQHGPKGEKGYPGIPGYCGPPGPPGPPGIPGNHGNNGNNGATGHEGAKGEKGDKGD
LGPRGERGQHGPKGEKGYPGIPDYKDDDDKLQISSTVAAAHHHHHHV
Jagged1 (hsJAG1)
(SEQ ID NO: 85)
MRSPRTRGRSGRPLSLLLALLCALRAKVCGASGQFELEILSMQNVNGELQ
NGNCCGGARNPGDRKCTRDECDTYFKVCLKEYQSRVTAGGPCSFGSGSTPVIGGNT
FNLKASRGNDRNRIVLPFSFAWPRSYTLLVEAWDSSNDTVQPDSIIEKASHSGMINPS
RQWQTLKQNTGVAHFEYQIRVTCDDYYYGFGCNKFCRPRDDFFGHYACDQNGNKT
CMEGWMGPECNRAICRQGCSPKHGSCKLPGDCRCQYGWQGLYCDKCIPHPGCVHG
ICNEPWQCLCETNWGGQLCDKDLNYCGTHQPCLNGGTCSNTGPDKYQCSCPEGYSG
PNCEIAEHACLSDPCHNRGSCKETSLGFECECSPGWTGPTCSTNIDDCSPNNCSHGGT
CQDLVNGFKCVCPPQWTGKTCQLDANECEAKPCVNAKSCKNLIASYYCDCLPGWM
GQNCDINLKEFMGYQGPPGPPGPPGIPGNHGNNGNNGATGHEGAKGEKGDKGDLGP
RGERGQHGPKGEKGYPGIPGYCGPPGPPGPPGIPGNHGNNGNNGATGHEGAKGEKG
DKGDLGPRGERGQHGPKGEKGYPGIPDYKDDDDKLQISSTVAAAHHHHHHV
Platelet-derived growth factor receptor A (hsPDGFRA)
(SEQ ID NO: 86)
MGTSHPAFLVLGCLLTGLSLILCQLSLPSILPNENEKVVQLNSSFSLRCFGE
SEVSWQYPMSEEESSDVEIRNEENNSGLFVTVLEVSSASAAHTGLYTCYYNHTQTEE
NELEGRHIYIYVPDPDVAFVPLGMTDYLVIVEDDDSAIIPCRTTDPETPVTLHNSEGVV
PASYDSRQGFNGTFTVGPYICEATVKGKKFQTIPFNVYALKATSELDLEMEALKTVY
KSGETIVVTCAVFNNEVVDLQWTYPGEVKGKGITMLEEIKVPSIKLVYTLTVPEATV
KDSGDYECAARQATREVKEMKKVTISVHEKGFIEIKPTFSQLEAVNLHEVKHFVVEV
RAYPPPRISWLKNNLTLIENLTEITTDVEKIQEIRYRSKLKLIRAKEEDSGHYTIVAQNE
DAVKSYTFELLTQVPSSILDLVDDHHGSTGGQTVRCTAEGTPLPDIEWMICKDIKKCN
NETSWTILANNVSNIITEIHSRDRSTVEGRVTFAKVEETIAVRCLAKNLLGAENRELKL
VAPLKEFMGYQGPPGPPGPPGIPGNHGNNGNNGATGHEGAKGEKGDKGDLGPRGE
RGQHGPKGEKGYPGIPGYCGPPGPPGPPGIPGNHGNNGNNGATGHEGAKGEKGDKG
DLGPRGERGQHGPKGEKGYPGIPDYKDDDDKLQISSTVAAAHHHHHHV
hsVEGFR1 domain 2-hsVEGFR2 domain 3 (hsV1V2)
(SEQ ID NO: 87)
MVSYWDTGVLLCALLSCLLLTGSSSGSKIFISDTGRPFVEMYSEIPEIIHMT
EGRELVIPCRVTSPNITVTLKKFPLDTLIPDGKRIIWDSRKGFIISNATYKEIGLLTCEAT
VNGHLYKTNYLTHRQGGGGSGGGGSLKIVVVVGYRIYDVVLSPSHGIELSVGEKLV
LNCTARTELNVGIDFNWEYPSSKHQHKKLVNRDLKTQSGSEMKKFLSTLTIDGVTRS
DQGLYTCAASSGLMTKKNSTFVRVHGGGGSGGGGSLKEFMGYQGPPGPPGPPGIPG
NHGNNGNNGATGHEGAKGEKGDKGDLGPRGERGQHGPKGEKGYPGIPGYCGPPGP
PGPPGIPGNHGNNGNNGATGHEGAKGEKGDKGDLGPRGERGQHGPKGEKGYPGIPD
YKDDDDKLQISSTVAAAHHHHHHV
TGF beta receptor 1 (hsTGFBR1)
(SEQ ID NO: 88)
MEAAVAAPRPRLLLLVLAAAAAAAAALLPGATALQCFCHLCTKDNFTCV
TDGLCFVSVTETTDKVIHNSMCIAEIDLIPRDRPFVCAPSSKTGSVTTTYCCNQDHCN
KIELPTTVKSLKEFMGYQGPPGPPGPPGIPGNHGNNGNNGATGHEGAKGEKGDKGD
LGPRGERGQHGPKGEKGYPGIPGYCGPPGPPGPPGIPGNHGNNGNNGATGHEGAKG
EKGDKGDLGPRGERGQHGPKGEKGYPGIPDYKDDDDKLQISSTVAAAHHHHHHV
TGF beta receptor 2 (hsTGFBR2)
(SEQ ID NO: 89)
MGRGLLRGLWPLHIVLWTRIASTIPPHVQKSVNNDMIVTDNNGAVKFPQL
CKFCDVRFSTCDNQKSCMSNCSITSICEKPQEVCVAVWRKNDENITLETVCHDPKLP
YHDFILEDAASPKCIMKEKKKPGETFFMCSCSSDECNDNIIFSEEYNTSNLKEFMGYQ
GPPGPPGPPGIPGNHGNNGNNGATGHEGAKGEKGDKGDLGPRGERGQHGPKGEKG
YPGIPGYCGPPGPPGPPGIPGNHGNNGNNGATGHEGAKGEKGDKGDLGPRGERGQH
GPKGEKGYPGIPDYKDDDDKLQISSTVAAAHHHHHHV
TGF beta receptor 3 (hsTGFBR3)
(SEQ ID NO: 90)
MTSHYVIAIFALMSSCLATAGPEPGALCELSPVSASHPVQALMESFTVLSG
CASRGTTGLPQEVHVLNLRTAGQGPGQLQREVTLHLNPISSVHIHHKSVVFLLNSPHP
LVWHLKTERLATGVSRLFLVSEGSVVQFSSANFSLTAETEERNFPHGNEHLLNWARK
EYGAVTSFTELKIARNIYIKVGEDQVFPPKCNIGKNFLSLNYLAEYLQPKAAEGCVMS
SQPQNEEVHIIELITPNSNPYSAFQVDITIDIRPSQEDLEVVKNLILILKCKKSVNWVIKS
FDVKGSLKIIAPNSIGFGKESERSMTMTKSIRDDIPSTQGNLVKWALDNGYSPITSYTM
APVANRFHLRLENNAEEMGDEEVHTIPPELRILLDPGALPALQNPPIRGGEGQNGGLP
FPFPDISRRVWNEEGEDGLPRPKDPVIPSIQLFPGLREPEEVQGSVDIALSVKCDNEKM
IVAVEKDSFQASGYSGMDVTLLDPTCKAKMNGTHFVLESPLNGCGTRPRWSALDGV
VYYNSIVIQVPALGDSSGWPDGYEDLESGDNGFPGDMDEGDASLFTRPEIVVFNCSL
QQVRNPSSFQEQPHGNITFNMELYNTDLFLVPSQGVFSVPENGHVYVEVSVTKAEQE
LGFAIQTCFISPYSNPDRMSHYTIIENICPKDESVKFYSPKRVHFPIPQADMDKKRFSFV
FKPVFNTSLLFLQCELTLCTKMEKHPQKLPKCVPPDEACTSLDASIIWAMMQNKKTF
TKPLAVIHHEAESKEKGPSMKEPNPISPPILKEFMGYQGPPGPPGPPGIPGNHGNNGN
NGATGHEGAKGEKGDKGDLGPRGERGQHGPKGEKGYPGIPGYCGPPGPPGPPGIPG
NHGNNGNNGATGHEGAKGEKGDKGDLGPRGERGQHGPKGEKGYPGIPDYKDDDD
KLQISSTVAAAHHHHHHV
Fibrinogen-like protein 1 (hsFGL1)
(SEQ ID NO: 91)
MAKVFSFILVTTALTMGREISALEDCAQEQMRLRAQVRLLETRVKQQQV
KIKQLLQENEVQFLDKGDENTVIDLGSKRQYADCSEIFNDGYKLSGFYKIKPLQSPAE
FSVYCDMSDGGGWTVIQRRSDGSENFNRGWKDYENGFGNFVQKHGEYWLGNKNL
HFLTTQEDYTLKIDLADFEKNSRYAQYKNFKVGDEKNFYELNIGEYSGTAGDSLAGN
FHPEVQWWASHQRMKFSTWDRDHDNYEGNCAEEDQSGWWFNRCHSANLNGVYY
SGPYTAKTDNGIVWYTWHGWWYSLKSVVMKIRPNDFIPNVILKEFMGYQGPPGPPG
PPGIPGNHGNNGNNGATGHEGAKGEKGDKGDLGPRGERGQHGPKGEKGYPGIPGY
CGPPGPPGPPGIPGNHGNNGNNGATGHEGAKGEKGDKGDLGPRGERGQHGPKGEK
GYPGIPDYKDDDDKLQISSTVAAAHHHHHHV

Exemplary fusion proteins comprising heterologous peptides that comprise receptor inhibiting protein (RIP) kinase inhibitors and extracellular matrix metalloproteinase inhibitors (“Timp”) are described herein. Where so indicated in the fusion protein name, the fusion protein does not contain a CCD, but rather contains an MSP sequence that is positioned at the C-terminus of the protein. In various embodiments, the disclosed fusion proteins comprise one or more heterologous peptides comprising an amino acid sequence that is at least 85%, at least 90%, at least 95%, at least 98%, at least 99%, or at least 99.5% identical to the amino acid sequences that follow. Any of the disclosed fusion proteins may differ relative to any of the following amino acid sequences by 1, 2, 3, 4, 5, 6, 7, 8, 9, 10, 10-15, 15-20, 20-30, 30-50, or more than 50 amino acids. These differences may comprise amino acids that have been inserted, deleted, or substituted relative to the sequence of any of SEQ ID NOs: 92-102. In some embodiments, the disclosed fusion proteins contain stretches of about 10, about 20, about 25, about 40, 50, about 75, about 100, about 125, about 150, about 175, or about 180 amino acids in common with the sequence of any of SEQ ID NOs: 92-102.

Many of the sequences that follow (SEQ ID NOs: 92-102) are illustrated with poly(A) tails and polyhistidine tags, or other tags, at the C terminus. However, in some embodiments, and as can readily be visualized by one skilled in the art, the disclosed exemplary fusion proteins do not contain these tags.

(“ms” designates mouse sequences):

Secretase-inactive Presenilin 1 D385A mutant (PS1D385A)
(SEQ ID NO: 92)
MTELPAPLSYFQNAQMSEDNHLSNTNDNRERQEHNDRRSLGHPEPLSNG
RPQGNSRQVVEQDEEEDEELTLKYGAKHVIMLFVPVTLCMVVVVATIKSVSFYTRK
DGQLIYTPFTEDTETVGQRALHSILNAAIMISVIVVMTILLVVLYKYRCYKVIHAWLII
SSLLLLFFFSFIYLGEVFKTYNVAVDYITVALLIWNFGVVGMISIHWKGPLRLQQAYLI
MISALMALVFIKYLPEWTAWLILAVISVYDLVAVLCPKGPLRMLVETAQERNETLFP
ALIYSSTMVWLVNMAEGDPEAQRRVSKNSKYNAESTERESQDTVAENDDGGFSEE
WEAQRDSHLGPHRSTPESRAAVQELSSSILAGEDPEERGVKLGLGAFIFYSVLVGKAS
ATASGDWNTTIACFVAILIGLCLTLLLLAIFKKALPALPISITFGLVFYFATDYLVQPFM
DQLAFHQFYI
TIMP metallopeptidase inhibitor 1 (msTimpl)
(SEQ ID NO: 93)
MMAPFASLASGILLLLSLIASSKACSCAPPHPQTAFCNSDLVIRAKFMGSPE
INETTLYQRYKIKMTKMLKGFKAVGNAADIRYAYTPVMESLCGYAHKSQNRSEEFLI
TGRLRNGNLHISACSFLVPWRTLSPAQQRAFSKTYSAGCGVCTVFPCLSIPCKLESDT
HCLWTDQVLVGSEDYQSRHFACLPRNPGLCTWRSLGARLKEFMGYQGPPGPPGPPG
IPGNHGNNGNNGATGHEGAKGEKGDKGDLGPRGERGQHGPKGEKGYPGIPGYCGP
PGPPGPPGIPGNHGNNGNNGATGHEGAKGEKGDKGDLGPRGERGQHGPKGEKGYP
GIPDYKDDDDKLQISSTVAAAHHHHHHV
TIMP metallopeptidase inhibitor 2 (msTimp2)
(SEQ ID NO: 94)
MGAAARSLRLALGLLLLATLLRPADACSCSPVHPQQAFCNADVVIRAKA
VSEKEVDSGNDIYGNPIKRIQYEIKQIKMFKGPDKDIEFIYTAPSSAVCGVSLDVGGKK
EYLIAGKAEGDGKMHITLCDFIVPWDTLSITQKKSLNHRYQMGCECKITRCPMIPCYI
SSPDECLWMDWVTEKSINGHQAKFFACIKRSDGSCAWYRGAAPPKQEFLDIEDPLKE
FMGYQGPPGPPGPPGIPGNHGNNGNNGATGHEGAKGEKGDKGDLGPRGERGQHGP
KGEKGYPGIPGYCGPPGPPGPPGIPGNHGNNGNNGATGHEGAKGEKGDKGDLGPRG
ERGQHGPKGEKGYPGIPDYKDDDDKLQISSTVAAAHHHHHHV
TIMP metallopeptidase inhibitor 3 (msTimp3)
(SEQ ID NO: 95)
MTPWLGLVVLLSCWSLGHWGAEACTCSPSHSQDAFCNSDIVIRAKVVGK
KLVTEGPFGTLVYTIKQMKMYRGFSKMPHVQYIHTEASESLCGLKLEVNKYQYLLT
GRVYEGKMYTGLCNFVERWDHLTLSQRKGLNYRYHLGCNCKINSCYYLPCFVTSK
NECLWTDMLSNFGYPGYQSKHYACIRQKGGYCSWYRGWAPPDKSISNATDPLKEF
MGYQGPPGPPGPPGIPGNHGNNGNNGATGHEGAKGEKGDKGDLGPRGERGQHGPK
GEKGYPGIPGYCGPPGPPGPPGIPGNHGNNGNNGATGHEGAKGEKGDKGDLGPRGE
RGQHGPKGEKGYPGIPDYKDDDDKLQISSTVAAAHHHHHHV
TIMP metallopeptidase inhibitor 4 (msTimp4)
(SEQ ID NO: 96)
MPWSPLAALSWALVLRLLALLWPPGRGEACSCAPAHPQQHFCHSALVIR
AKISSEKVVPASKDPADTQKLIRYEIKQIKMFKGFEKAKDIQYVYTPFDSSLCGVKLE
TNSHKQYLLTGQILSDGKVFIHLCNYIEPWEDLSLVQRESLNHHYHQNCGCQITTCY
AVPCTISAPNECLWTDWLLERKLYGYQAQHYVCMKHVDGICSWYRGHLHLRKEYV
DIIQPLKEFMGYQGPPGPPGPPGIPGNHGNNGNNGATGHEGAKGEKGDKGDLGPRG
ERGQHGPKGEKGYPGIPGYCGPPGPPGPPGIPGNHGNNGNNGATGHEGAKGEKGDK
GDLGPRGERGQHGPKGEKGYPGIPDYKDDDDKLQISSTVAAAHHHHHHV
msTimp1-MSP
(SEQ ID NO: 97)
MMAPFASLASGILLLLSLIASSKACSCAPPHPQTAFCNSDLVIRAKFMGSPE
INETTLYQRYKIKMTKMLKGFKAVGNAADIRYAYTPVMESLCGYAHKSQNRSEEFLI
TGRLRNGNLHISACSFLVPWRTLSPAQQRAFSKTYSAGCGVCTVFPCLSIPCKLESDT
HCLWTDQVLVGSEDYQSRHFACLPRNPGLCTWRSLGARTRTRPLEQKLISEEDLAAN
DDGIFCSSSNFLGISFLLILMLILYSFI
msTimp2-MSP
(SEQ ID NO: 98)
MGAAARSLRLALGLLLLATLLRPADACSCSPVHPQQAFCNADVVIRAKA
VSEKEVDSGNDIYGNPIKRIQYEIKQIKMFKGPDKDIEFIYTAPSSAVCGVSLDVGGKK
EYLIAGKAEGDGKMHITLCDFIVPWDTLSITQKKSLNHRYQMGCECKITRCPMIPCYI
SSPDECLWMDWVTEKSINGHQAKFFACIKRSDGSCAWYRGAAPPKQEFLDIEDPTRT
RPLEQKLISEEDLAANDDGIFCSSSNFLGISFLLILMLILYSFI
msTimp3-MSP
(SEQ ID NO: 99)
MTPWLGLVVLLSCWSLGHWGAEACTCSPSHSQDAFCNSDIVIRAKVVGK
KLVTEGPFGTLVYTIKQMKMYRGFSKMPHVQYIHTEASESLCGLKLEVNKYQYLLT
GRVYEGKMYTGLCNFVERWDHLTLSQRKGLNYRYHLGCNCKINSCYYLPCFVTSK
NECLWTDMLSNFGYPGYQSKHYACIRQKGGYCSWYRGWAPPDKSISNATDPTRTRP
LEQKLISEEDLAANDDGIFCSSSNFLGISFLLILMLILYSFI
msTimp4-MSP
(SEQ ID NO: 100)
MPWSPLAALSWALVLRLLALLWPPGRGEACSCAPAHPQQHFCHSALVIR
AKISSEKVVPASKDPADTQKLIRYEIKQIKMFKGFEKAKDIQYVYTPFDSSLCGVKLE
TNSHKQYLLTGQILSDGKVFIHLCNYIEPWEDLSLVQRESLNHHYHQNCGCQITTCY
AVPCTISAPNECLWTDWLLERKLYGYQAQHYVCMKHVDGICSWYRGHLHLRKEYV
DIIQPTRTRPLEQKLISEEDLAANDDGIFCSSSNFLGISFLLILMLILYSFI

Exemplary CCD fusion proteins of the disclosure (monomeric) are described herein. In various embodiments, the disclosed fusion proteins comprise an amino acid sequence that is at least 85%, at least 90%, at least 95%, at least 98%, at least 99%, or at least 99.5% identical to SEQ ID NOs: 56, 57, 59, 70, 72, 77, 80, 87, 101, 102, or 105-108. In particular embodiments, the disclosed fusion proteins comprise the sequence of any one of SEQ ID NOs: 56, 57, 59, 70, 72, 77, 80, 87, 101, 102, or 105-108. In certain embodiments, the disclosed fusion proteins comprise the sequence of SEQ ID NO: 57. In certain embodiments, the disclosed fusion proteins comprise the sequence of any of one of SEQ ID NOs: 105-108.

Exemplary fusion proteins comprising two heterologous peptides include IcosL-CCD-TRAIL and FGFR1-CCD-VEGFR1-VEGFR2 (“FGFR1-CCD-V1V2”), which are exemplified in the following sequences:

hIcosL-CCD-hTRAIL
(SEQ ID NO: 101)
MRLGSPGLLFLLFSSLRADTQEKEVRAMVGSDVELSCACPEGSRFDLNDV
YVYWQTSESKTVVTYHIPQNSSLENVDSRYRNRALMSPAGMLRGDFSLRLFNVTPQ
DEQKFHCLVLSQSLGFQEVLSVEVTLHVAANFSVPVVSAPHSPSQDELTFTCTSINGY
PRPNVYWINKTDNSLLDQALQNDTVFLNMRGLYDVVSVLRIARTPSVNIGCCIENVL
LQQNLTVGSQTGNDIGERDKITENPVLKEFGGGGSGGGGSGGGGSMGYQGPPGPPG
PPGIPGNHGNNGNNGATGHEGAKGEKGDKGDLGPRGERGQHGPKGEKGYPGIPGY
CGPPGPPGPPGIPGNHGNNGNNGATGHEGAKGEKGDKGDLGPRGERGQHGPKGEK
GYPGIPDYKDDDDKGGGGSGGGGSGGGGSLQISTRISTVQEKQQNISPLVRERGPQR
VAAHITGTRGRSNTLSSPNSKNEKALGRKINSWESSRSGHSFLSNLHLRNGELVIHEK
GFYYIYSQTYFRFQEEIKENTKNDKQMVQYIYKYTSYPDPILLMKSARNSCWSKDAE
YGLYSIYQGGIFELKENDRIFVSVTNEHLIDMDHEASFFGAFLVGTRAAAHHHHHHV
hsFGFR1-CCD-hsVEGFR1-hsVEGFR2
(SEQ ID NO: 102)
MWSWKCLLFWAVLVTATLCTARPSPTLPEQAQPWGAPVEVESFLVHPGD
LLQLRCRLRDDVQSINWLRDGVQLAESNRTRITGEEVEVQDSVPADSGLYACVTSSP
SGSDTTYFSVNVSDALPSSEDDDDDDDSSSEEKETDNTKPNPVAPYWTSPEKMEKKL
HAVPAAKTVKFKCPSSGTPNPTLRWLKNGKEFKPDHRIGGYKVRYATGSIIMDSVVP
SDKGNYTCIVENEYGSINHTYQLDVVERSPHRPILQAGLPANKTVALGSNVEFMCKV
YSDPQPHIQWLKHIEVNGSKIGPDNLPYVQILKTAGVNTTDKEMEVLHLRNVSFEDA
GEYTCLAGNSIGLSHHSAWLTVLEALEERPAVMLKEFMGYQGPPGPPGPPGIPGNHG
NNGNNGATGHEGAKGEKGDKGDLGPRGERGQHGPKGEKGYPGIPGYCGPPGPPGPP
GIPGNHGNNGNNGATGHEGAKGEKGDKGDLGPRGERGQHGPKGEKGYPGIPDYKD
DDDKLQISGGGGSGGGGSIFISDTGRPFVEMYSEIPEIIHMTEGRELVIPCRVTSPNITV
TLKKFPLDTLIPDGKRIIWDSRKGFIISNATYKEIGLLTCEATVNGHLYKTNYLTHRQG
GGGSGGGGSLKIVVVVGYRIYDVVLSPSHGIELSVGEKLVLNCTARTELNVGIDFNW
EYPSSKHQHKKLVNRDLKTQSGSEMKKFLSTLTIDGVTRSDQGLYTCAASSGLMTK
KNSTFVRVHAAAHHHHHHV

Any of the disclosed fusion proteins and compositions thereof may have a long shelf-life, e.g., have stability after storage for up to a particular time point, e.g. one week to 10 years, at a certain temperature, such as −20° C. or room temperature. The disclosed compositions are considered to be stable at a particular time point if there is an absence of significant change of appearance in Western blot images performed after SDS-PAGE analysis of the protein sample before and after storage, e.g., an absence of “new” protein bands in the Western blot that may indicate the presence of degradants.

Accordingly, in certain embodiments, at least 95%, often at least 98%, and often 100% of individual aliquots of the disclosed fusion proteins (e.g., unit doses), or compositions thereof, are stable after 8 months to 10 years of storage at about −20° C. In certain embodiments, at least 95%, often at least 98%, and usually 100% of individual aliquots of the disclosed compositions exhibit stability after several weeks or months (e.g., after 3 months, after 6 months, after 12 months, or after 24 months) of storage at about −20° C., as detected by SDS-PAGE.

In specific embodiments, at least 95%, often at least 98%, and often 100% of individual aliquots of the disclosed compositions are stable after several weeks to months of storage at about −20° C., followed by a thaw to a temperature of about 2° C. to about 8° C. (e.g., about 4 or 4.5° C.), followed by storage at one to six weeks at about 2° C. to about 8° C. (e.g., about 4 or 4.5° C.). In certain embodiments, at least 95%, often at least 98%, and often 100% of individual aliquots of the disclosed compositions are stable after several weeks to months of storage at about −20° C., followed by a thaw to and storage for about 2 weeks, 3 weeks, 4 weeks, 5 weeks, 6 weeks, 7 weeks, or 8 weeks at about 2° C. to about 8° C. (e.g., 4.0° C. or 4.5° C.). In certain embodiments, at least 95%, often at least 98%, and often 100% of individual aliquots of the disclosed compositions are stable after freezing for a length of time between six months and ten years, and then thawed to and stored for at least one month, two months, three months, four months, six months, eight months, ten months, twelve months, fifteen months, eighteen months, twenty months, twenty-two months, or twenty-four months at about 2° C. to about 8° C. (e.g., 4.0° C. or 4.5° C.).

In certain embodiments, the solution is stored long-term at temperatures less than −20° C. For instance, the solution may be stored at temperatures of −30° C., −40° C., −60° C. or −80° C.

### Multimeric Fusion Proteins

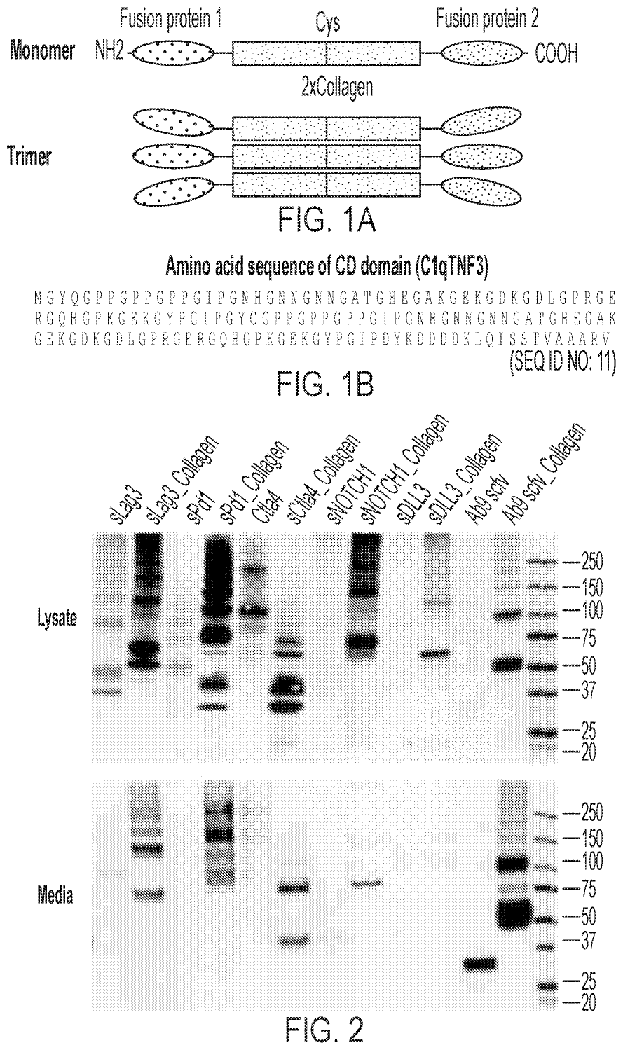

To enhance the formation of multimers of the CCD and fusion protein, two CCD domains from C1qTNF3 were connected by a glycine-tyrosine-cysteine linker. Without being restricted to a single theory, the cysteine residues between adjoining CCD domains form intermolecular disulfide bonds (or disulfide bridges). Depending on the nature of the heterologous peptide of interest, the protein is incorporated on the N-terminus or C-terminus through molecular cloning techniques known in the art. In some cases, heterologous peptides are incorporated on each terminus of the CCD domain to form a bi-functional molecule, sometimes known as a “double warhead” (see FIG. 1A).

The cDNA encoding CCD fusion protein may be inserted into an rAAV vector by genetic engineering techniques. The complete rAAV vectors with protein of interest cDNA may subsequently be used for enhanced expression of the heterologous peptide.

Accordingly, in some aspects, provided herein are multimeric fusion proteins comprising two, three, four, six or eight CCD fusion protein monomers. In particular embodiments, the multimeric fusion protein comprises a trimer or a hexamer. The monomers of the multimeric fusion protein may be the same, or they may be different. Exemplary multimeric proteins in which the monomers may be different may arise by the delivery of fusion proteins comprising native and optimized C1qTNF3 collagen domains, wherein these domains associate (e.g. into trimers) within the target cell due to the presence of Gly-X-Y repeats.

In particular embodiments of the disclosed multimeric fusion proteins, the CCDs of the two or more monomers are linked to each other by one or more glycine-cysteine-glycine linker domains or glycine-tyrosine-cysteine linker domains. In particular embodiments, the two or more monomers are linked to each other by one or more disulfide bridges.

In some aspects, the disclosed multimeric fusion proteins have bispecificity for two or more ligands. In exemplary aspects, the multimeric fusion protein has a binding avidity for two or more ligands in vivo that is substantially equivalent to the sum of the binding affinities of the first and second heterologous peptides for the same ligands in vivo. In some aspects, the multimeric fusion protein has a binding avidity for two or more ligands in vivo that is greater than the sum of the binding affinities of the first and second heterologous peptides for the same ligands in vivo. The maintenance or enhancement of avidity in vivo of the disclosed multimeric fusion proteins of represents an unexpected and important result of the invention.

There is increasing evidence that single molecular targeting of immune checkpoint molecules at the immune synapse, or modulators thereof, may be insufficient for desired therapies targeting immune checkpoints. The multimeric fusion proteins described herein address a need in the art for development of bi- and multi-specific therapies for immunomodulation in Alzheimer's Disease, cancer or autoimmune disorders and conditions. Accordingly, described herein are bispecific functional multimeric fusion proteins that target tau protein and amyloid beta peptide.

In some embodiments, the second heterologous peptide of any of the disclosed fusion proteins comprises an scFv having affinity for an amyloid beta peptide (Aβ). In some embodiments, the first heterologous peptide may comprise an scFv having affinity for a tau protein (α-tau). In other embodiments, the first heterologous peptide may comprise a glucocerebrosidase (GBA). In particular embodiments, the first heterologous peptide comprises an scFv having affinity for a tau protein (α-tau) and the second heterologous peptide comprises an scFv having affinity for an amyloid beta peptide (Aβ). Exemplary amyloid beta scFv peptides include scFv's against beta peptide 9 and beta peptide 5.

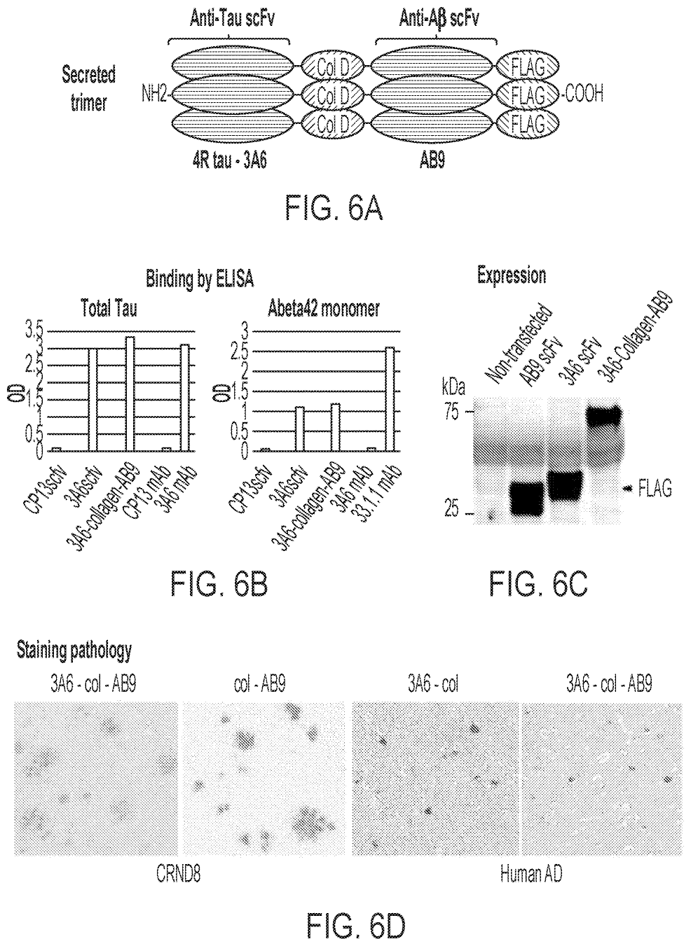

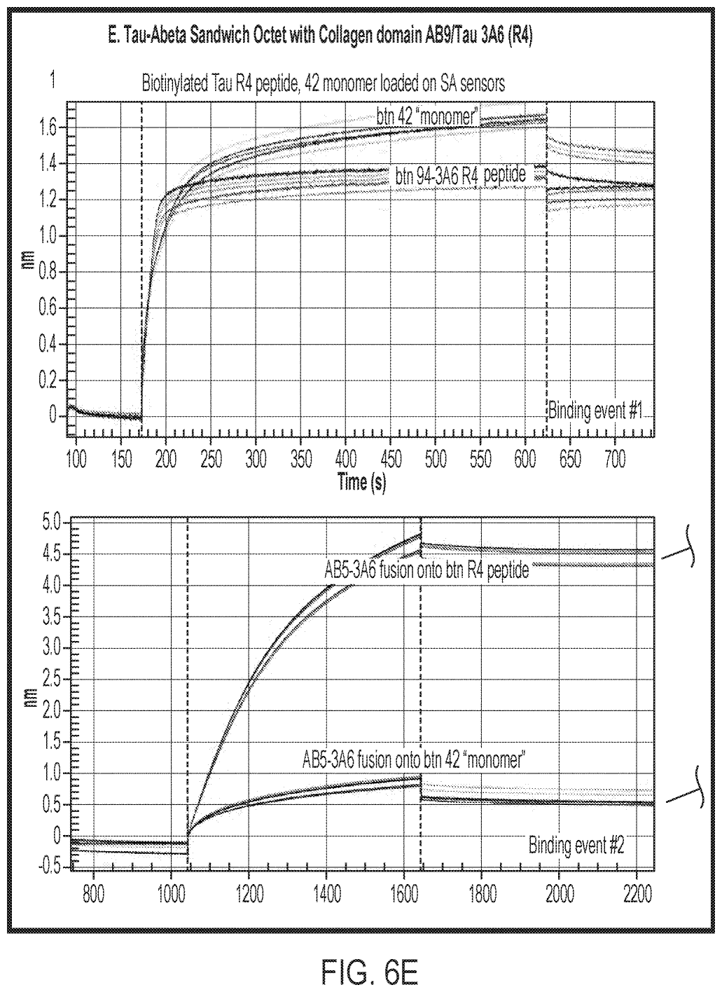

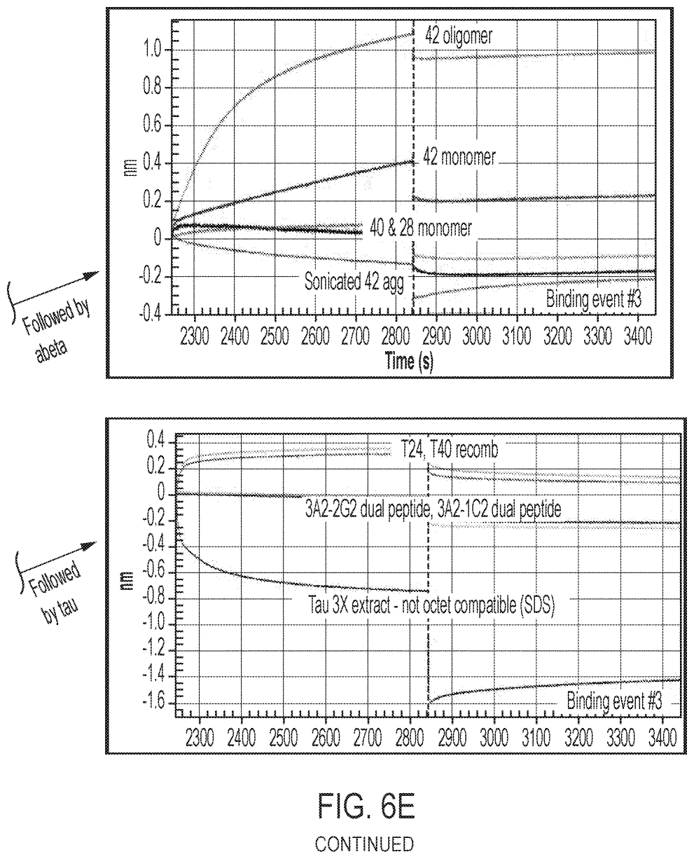

In particular embodiments, the scFv targeting tau is a fragment from a 94-3A6 anti-tau antibody, and the scFv targeting Aβ has specificity for beta peptide 9 (“Aβ9 scFv”) (see FIGS. 6A-6E). See Croft C L et al. (2018), Novel monoclonal antibodies targeting the microtubule binding domain of human tau, PLoS ONE 13(4):e0195211, herein incorporated by reference. These fusion proteins may herein be referred to as “nanobodies targeting tau and Aβ.” As shown in the Examples that follow, these nanobodies demonstrate avidity to tau and Aβ in vitro and in vivo. These nanobodies exhibit avidity to these ligands in vivo to a degree that is substantially equivalent to the sum of the binding affinities of α-tau scFv and α-AβscFv for the same ligands in vivo. In some embodiments, the first heterologous peptide of the fusion protein comprises a GBA protein, and the second heterologous peptide comprises an scFv having affinity for an amyloid beta peptide 9 (Aβ9).

Binding affinity and avidity of the heterologous peptides of the disclosure (such as scFv's) in vitro can be determined by a variety of methods including equilibrium dialysis, equilibrium binding, gel filtration, ELISA, surface plasmon resonance, or spectroscopy (e.g., using a fluorescence or FACS assay). Binding affinity and avidity in vivo can be inferred, e.g., by activity in a functional in vivo assay such as a cell metabolic activity assay or end-product detection assay such as an amyloid plaque detection assay; or directly by methods including in vivo imaging in a subject, e.g., immunohistochemistry (IHC), bioluminescence imaging (BLI), Magnetic Resonance Imaging (MRI), positron emission tomography (PET), electron microscopy, X-ray computed tomography, Raman imaging, optical coherence tomography, absorption imaging, thermal imaging, fluorescence reflectance imaging, fluorescence microscopy, fluorescence molecular tomographic imaging, nuclear magnetic resonance imaging, X-ray imaging, ultrasound imaging, photoacoustic imaging, lab assays, or in any situation where tagging/staining/imaging is required.

The fusion proteins comprising an scFv may comprise the structure NH₂-[α-tau scFv]-[CCD]-[α-Aβ scFv]-COOH, NH₂-[α-tau scFv]-[CCD]-[α-Aβ scFv]-FLAG-COOH, NH₂-[GBA]-[CCD]-[α-Aβ scFv]-COOH, NH₂-FLAG-[α-tau scFv]-[CCD]-[α-Aβ scFv]-COOH, NH₂-[GBA]-[CCD]-[α-Aβ scFv]-COOH, or NH₂-[α-Aβ scFv]-[CCD]-[GBA]-COOH, wherein each instance of “14” indicates the presence of an optional linker sequence. In some embodiments, “α-Aβ scFv” represents α-Aβ9 scFv. In certain embodiments, these multimeric fusion proteins are trimeric or hexameric (see FIG. 6A).

In other exemplary embodiments, the first heterologous peptide comprises an scFv having affinity for an amyloid beta peptide (Aβ) and the second heterologous peptide comprises an scFv having affinity for a different amyloid beta peptide. Surprisingly, as shown in the Example 4 that follows, the administration of an rAAV vector encoding a monomeric nanobody wherein the first heterologous peptide comprised an scFv having affinity for an Aβ9 and the second heterologous peptide comprised an scFv having affinity for a different amyloid precursor peptide successfully reduced amyloid plaque deposition in the brains of CRDN8 mice relative to administration of unconjugated scFv's in solution (see FIG. 6D). Based on the amyloid plaque reduction observed in this functional in vivo assay, it can be inferred that this monomeric nanobody has enhanced in vivo avidity to Aβ9 and amyloid precursor peptide than the same scFv's in an unconjugated state.

In particular embodiments, the multimeric fusion protein has an avidity for its ligand of greater than 10⁷M⁻¹. In certain embodiments, the multimeric protein has an avidity for its ligand of greater than 10⁸M⁻¹. In certain embodiments, multimeric protein has an avidity for its ligand of greater than 10⁹M⁻¹. In certain embodiments, the multimeric protein has an avidity for its ligand between 10⁷M⁻¹ and 10⁹M⁻¹, between 10⁷M⁻¹ and 10⁸M⁻¹, between 10⁸ M⁻¹ and 10¹⁰ M⁻¹, between 10⁸M⁻¹ and 10⁹M⁻¹, and between 10⁹M⁻¹ and 10¹⁰ M⁻¹.

In a particular embodiment, each monomer of the multimeric fusion protein comprises a CCD comprising at least 10 G-X-Y repeats; wherein G is glycine, X is any amino acid, and Y is any amino acid, wherein at least 10 of the G-X-Y repeats are G-P-P or G-P-O, wherein at least 6 of the G-X-Y repeats are G-P-O, and wherein P is proline and O is hydroxyproline; and an antibody domain; wherein the hydroxyprolinated collagen-like domains of each one monomer interact with one another to form a multimeric protein that binds to a ligand with an avidity of at least 10⁷ M⁻¹

The fusion protein can include, or exclude, the sequence of affinity tags for the purpose of detection and purification of the fusion proteins of the disclosure. Examples of affinity tags include polyhistidine-tag (His₆ tag), myc-tag, Strep-tag, FLAG tag, E-tag, hemagglutin tag, T7, S-tag, HSV, VSV-G, anti-Xpress, and VS-tag.

Based on the specificity of the binding domains of the heterologous peptides, the fusion proteins described herein may be used for treating various disorders, including cancers, inflammation diseases, metabolic diseases, fibrosis diseases, and cardiovascular diseases. The invention accordingly features methods of treating such a disorder, e.g., by administering to a subject in need thereof an effective amount of a protein complex of the invention to treat the disorder. Subjects to be treated can be identified as having, or being at risk for acquiring, a condition characterized by the disorder. These methods may be performed alone or in conjunction with other drugs or therapy.

Because of the multi-specific binding affinity of fusion proteins described herein, one of skill in the art may use these fusion proteins to bridge molecules or cells that are normally are not associated with each other. This feature is particularly useful for cell-based therapies. In one example, one heterologous domain in the protein complex is capable of activating cytotoxic cells (e.g., cytotoxic T cells) by specifically binding to an effector antigen on the cytotoxic cells, while another heterologous domain specifically binds to a target antigen on a pathogen cell or a malignant cell to be destroyed. In this way, the protein complex can treat a disorder caused by the pathogen or malignant cells.

### C1q Proteins

C1q is a subunit of the C1 enzyme complex of the classical pathway of complement activation. It is composed of nine disulfide-linked dimers of the chains A, B and C, which share a common structure which consist of a N-terminal nonhelical region, a triple helical (collagenous) region and a C-terminal globular head which is called the C1q domain (Smith et al. 1994 Biochem. J. 301:249-256). Members of the C1q and TNF superfamily are involved in host defense, inflammation, apopotosis, autoimmunity, cell differentiation, organogenesis, hibernation and insulin-resistant obesity. Five strictly conserved residues have been identified in the Cc1q family (Kishore et al. Trends in Immunology 2004. 25(10):551-561). Each C1q domain exhibits a ten-stranded β-sandwich fold with a jelly-roll topology, consisting of two five-stranded β-sheets (A′, A, H, C, F) and (B′, B, G, D, E), each made of antiparallel strands. Each of the five conserved residues within c1q family proteins belongs to the hydrophobic core of the C1q domain. The β-strands are strongly conserved in the different C1q domains (relative to orientation and size), in contrast with the loops connecting the β-strands which exhibit significant variability. There are two well conserved regions within the C1q domain: an aromatic motif is located within the first half of the domain, the other conserved region is located near the C-terminal extremity.

In addition, the proteins C1q, collagen a1 (X), a2 (VII), the overwintering protein ACRP30, the inner ear structure protein, cerebellin and multimerin are classed as the protein family under the term C1q family, owing to their sequence homologies in their respective multimerizing sequence segments (Kischore and Reid, Immunopharmacol., 42: 15-21 (1999)), and, owing to their structure, these proteins are in the form of higher aggregates of, for example, trimers. Among the proteins with multimerization properties which are found in this family, for example the structure of the protein C1q, which is known from the complement system, is characterized by monomers, each of which has a globular domain which is known as the “head” and a “collagenaceous” helical sequence segment. It is this helical sequence segment, which forms a coiled-coil triple helix, via which the monomers trimerize. In turn, six of these C1q trimers form an oligomer, the oligomerization of the protein trimers, in turn, being based on interactions between the individual coiled-coil triple helices. The result of this structural arrangement of the protein or the multi-(oligo-)merized protein complex C1q is a construction also termed “bouquet”, it being ensured that globular, C-terminally arranged “head” domains are connected to give a hexamer of trimers.

The C1q and TNF family proteins have similar gene structures: their C1q and TNF homology domains are each encoded within one exon, whereas introns in both families are restricted to respective N-terminal collagen or stalk regions. The jelly-roll structure is remarkably similar to the capsid proteins of plant viruses and mammalian picoranviruses including foot- and-mouth virus and poliovirus.

The C-terminal globular domain of the C1q subcomponents and collagen types VIII and X is important both for the correct folding and alignment of the triple helix and for protein-protein recognition events. For collagen type X it has been suggested that the domain is important for initiation and maintenance of the correct assembly of the protein (Kwan et al. 1991 J. Cell Biol. 114:597-604). In adiponectin, the C1q domain can ameliorate hyperglycemia and hyperinsulinemia much more potently than full-length adiponectin. Adiponectin was shown to suppress mature macrophage function by significantly inhibiting their phagocytic activity and their LPS-induced production of TNF-α, and thus might resolve inflammation. Adiponectin has also been shown to reverse insulin resistance associated with obesity by decreasing triglyceride content in the muscle and liver of obese mice. Decreased adiponectin has been implicated in the development of insulin resistance in mouse models of obesity and type 2 diabetes. A mild autosomal disorder associated with growth plate abnormalities, called “Schmid metaphyseal chondrodysplasia” has been associated with missense mutations in the C1q domain of collagen X which disrupt the hydrophobic core and perturb trimer assembly. Specific mutations in the C1q domain of CTRP5 have been associated with late-onset retinal degeneration.

The collagen domain is found in collagens that are generally extracellular structural proteins involved in formation of connective tissue structure. The domain contains 20 copies of the G-X-Y repeat that forms a triple helix. The first position of the repeat is glycine, the second and third positions can be any residue but are frequently proline and hydroxyproline. Collagens are post translationally modified by proline hydroxylase to form the hydroxyproline residues. Defective hydroxylation is the cause of scurvy. Some members of the collagen superfamily are not involved in connective tissue structure but share the same triple helical structure. The antiproliferative (G1 mitotic arrest) and proapoptotic effect of C1q on human fibroblasts is mediated by the collagen region, via the calreticulin-CCD91 complex. This interaction enhances p38 MAPK activation, NF-κB activity and production of prooinflammatory cytokines and chemokines in macrophages.

Alteration of the activity of C1q domain containing proteins thus provides a means to alter disease phenotype and as such, identification of novel proteins of this type is highly relevant as they may play a role in or be useful in the development of treatments for the diseases identified above, as well as other disease states.

### Multimerization Strategies

Monovalent antibody fragments, e.g. single chain variable fragments (scFv) and nanobodies (VHHs) generally suffer from short circulation time due to fast renal clearance. Though engineering of these antibody fragments can yield high-affinity binding domains, strong binding capacity and a high local density of antigens can also lead to a decrease in tissue penetration when it comes to a situation called “binding site barrier”.

In the non-equilibrium environment of human tissue, monovalent antibody fragments suffer from moderate target retention due to the fast dissociation of the binding domains. In an optimal therapeutic molecule, an appropriate compromise must be established between optimal tumor penetration (considering molecular weight), functional affinity, and reasonable serum half-life. Enhancement of the binding strength of target binding molecules without compromising their antigen specificity is a challenging task.

To that end, small monovalent binding molecules can be engineered towards the intrinsic multivalent format of antibodies. Thus, the target binding strength of small antibody fragments can be significantly improved by increasing the number of antigen binding sites. Multimerization additionally leads to a prolonged circulation time due to the higher molecular weight and longer retention time at target tissues caused by the forced proximity of the binding moieties and slower K_{D} off-rates. The improved half-life and enhanced functional affinity allows the usage of lower doses in the clinic, which results in reduced costs and dose-dependent side effects.

Target binding molecules can be fused to multivalent scaffold molecules to achieve multivalency. Fusion to multivalent scaffolds has several advantages, among them the prolonged target residence time which can be achieved when multiple ligands bind to their target site due to the forced proximity of the ligands. In this case, a bound ligand brings the residual scaffold-associated counterparts into close proximity to the binding sites on the targeted cell, thus favoring both their binding and the rebinding of dissociated ligands. This of course strongly depends on the distribution of targeted molecules in close proximity.

As used herein, the term “scaffold” refers to a molecule of synthetic or natural origin with a defined architecture for the multivalent display of functional units. Scaffold molecules applied for multimerization may possess certain optimal attributes: small size, defined architecture, high stability, low tendency to aggregate, sufficient solubility in aqueous solutions and modification sites that can be addressed without affecting the integrity of the scaffold. Fusion to oligomerization domains has been found to increase the half-life of the fusion partners, thus enabling their activity for a prolonged period. The activity of multimeric functional biomolecules is enhanced because of a stronger target binding due to avidity, rather than affinity.

As an example of a scaffold, the human cartilage oligomeric matrix protein (COMP48, or “COMP”), which is composed of at least 46 residues capable of self-assembling into a parallel disulfide-stabilized pentameric structure, has previously been used for the pentamerization of various target-binding molecules. COMP plays a role in the structural integrity of cartilage via its interaction with other extracellular matrix proteins, such as collagen and fibronectin. A VHH targeting the melanoma peptide HLAA2 complex was fused to the N-terminus of COMP48 resulting in a pentameric antibody COMBODY that, compared to the monovalent VHH, exhibited an enhanced binding capacity.

Furthermore, a bispecific COMBODY was generated by additional fusion of an anti-CD3 scFv to the C-terminus. The expected bispecific decavalent molecule accumulated to larger aggregates that mediated specific cell lysis of peptide-loaded target cells. Another group generated a pentameric COMP-EGF fusion protein bearing 5 EGF ligands that specifically bound to the extracellular domain of EGFR on EGFR-overexpressing cancer cells and induced apoptosis. This fusion protein was produced in E. coli cells but needed extensive refolding due to the complex tertiary structure.

### Linkers and Signal Sequences

In various embodiments of the disclosed fusion proteins, linkers may be used to link any of the peptide domains. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is an amino acid or a plurality of amino acids (e.g., a peptide or protein). In some embodiments, the linker is a bond (e.g., a covalent bond), an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated.

In some embodiments, a linker comprises the amino acid sequence GGGS (SEQ ID NO: 103) or GGGSGGGS (SEQ ID NO: 104).

In various embodiments, the disclosed fusion proteins comprise a secretion signal sequence. The ability for cells to make and secrete extracellular proteins is central to many biological processes. Enzymes, growth factors, extracellular matrix proteins and signalling molecules are all secreted by cells. This is through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle. Polypeptides that are targeted to the secretory vesicles are either secreted into the extracellular matrix or are retained in the plasma membrane. The polypeptides that are retained in the plasma membrane will have one or more transmembrane domains.

### Pharmaceutical Compositions and Methods of Treatment

Further provided herein are pharmaceutical compositions that comprise a modified rAAV vector as disclosed herein, and further comprise a pharmaceutical excipient, and may be formulated for administration to host cell ex vivo or in situ in an animal, and particularly a human. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions, diseases or disorders as described herein.

The term “excipient” refers to a diluent, adjuvant, carrier, or vehicle with which the rAAV particle or preparation, or nucleic acid segment is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

In certain embodiments, the present disclosure provides a method of reducing AAV immunity in a subject, wherein the method further comprises administering to the subject a composition comprising the disclosed rAAV particles and a pharmaceutically acceptable excipient, optionally wherein the subject has been previously administered a composition comprising rAAV particles. In particular embodiments, the subject is a human.

In some embodiments, the number of rAAV particles administered to a host cell may be on the order ranging from 500 to 5,000 vector genomes (vgs)/cell. In particular embodiments, the disclosed methods comprise administration of rAAV particles in doses of about 500 vgs/cell, 1000 vgs/cell, 2000 vgs/cell, 3000 vgs/cell, 4000 vgs/cell, 5000 vgs/cell, 6000 vgs/cell or 7000 vgs/cell.

In some embodiments, the number of rAAV particles administered to a subject may be on the order ranging from 10⁶ to 10¹⁴ particles/mL or 10³ to 10¹³ particles/mL, or any values therebetween for either range, such as for example, about 10⁶, 10⁷, 10⁸, 10⁹, 10¹⁰, 10¹¹, 10¹², 10¹³, or 10¹⁴ particles/mL. In one embodiment, rAAV particles of higher than 10¹³ particles/mL are be administered. In some embodiments, the number of rAAV particles administered to a subject may be on the order ranging from 10⁶ to 10¹⁴ vgs/mL or 10³ to 10¹⁵ vgs/mL, or any values there between for either range, such as for example, about 10⁶, 10⁷, 10⁸, 10⁹, 10¹⁰, 10¹¹, 10¹², 10¹³, or 10¹⁴ vgs/mL. In certain embodiments, the disclosed methods comprise administration of rAAV particle compositions in doses of 3×10³-1×10⁴ vgs/mL. In one embodiment, rAAV particles of higher than 10¹³ vgs/mL are be administered.

The rAAV particles can be administered as a single dose, or divided into two or more administrations as may be required to achieve therapy of the particular disease or disorder being treated. In some embodiments, 0.0001 mL to 10 mL are delivered to a subject. In some embodiments, interferon-γ is co-administered with the rAAV particles. In some embodiments, interferon-γ is administered after administration of the rAAV particles.

In some embodiments, the disclosure provides formulations of compositions disclosed herein in pharmaceutically acceptable solutions for administration to a cell or an animal, either alone or in combination with one or more other modalities of therapy, and in particular, for therapy of human cells, tissues, and diseases affecting man.

If desired, rAAV particle or preparation and nucleic acid segments may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles or preparations and nucleic acid segments may thus be delivered along with various other agents as required in the particular instance. Such compositions may be purified from host cells or other biological sources, or alternatively may be chemically synthesized as described herein. As used herein, the term “vector” can refer to a nucleic acid segment (e.g., a plasmid or recombinant viral genome) or a viral vector (e.g., an rAAV particle comprising a recombinant genome).

Formulation of pharmaceutically-acceptable excipients is well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

Typically, these formulations may contain at least about 0.1% of the therapeutic agent (e.g., rAAV particle or preparation and/or nucleic acid segment) or more, although the percentage of the active ingredient(s) may, of course, be varied and may conveniently be between about 1 or 2% and about 70% or 80% or more of the weight or volume of the total formulation. Naturally, the amount of therapeutic agent(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

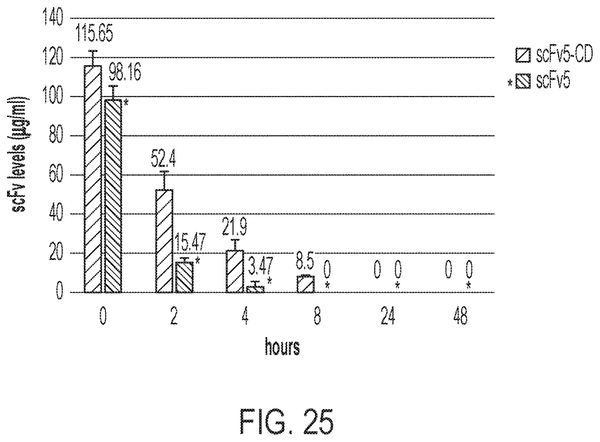

The half-life of an exemplary multimeric fusion protein, a CCD fusion with a single-chain variable fragment of amyloid beta protein, or α-Aβ5 scFv-CCD, was determined to be approximately 2 hours. 250 μg of purified biotinylated anti-pan Aβ5 scFv5 or pan Aβ5 scFv-CCD were injected intraperitoneally into adult non-transgenic B6C3F1 mice. Plasma was collected from the mice at various time points following the injection and levels of circulating scFvs were detected by a direct ELISA method with monomeric Aβ (10 μg/ml) as capture and Neutravidin-HRP secondary antibody as detection. Results are shown in FIG. 25.

In certain circumstances it will be desirable to deliver the rAAV particles or preparations and/or nucleic acid segments in suitably formulated pharmaceutical compositions disclosed herein either subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

The pharmaceutical forms of the compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The pharmaceutical compositions of the present disclosure can be administered to the subject being treated by standard routes including, but not limited to, pulmonary, intranasal, oral, inhalation, parenteral such as intravenous, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural and intrasternal injection.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or preparations and/or nucleic acid segments, in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Ex vivo delivery of cells transduced with rAAV particles or preparations is also contemplated herein. Ex vivo gene delivery may be used to transplant rAAV-transduced host cells back into the host. A suitable ex vivo protocol may include several steps. For example, a segment of target tissue or an aliquot of target fluid may be harvested from the host and rAAV particles or preparations may be used to transduce a nucleic acid segment into the host cells in the tissue or fluid. These genetically modified cells may then be transplanted back into the host. Several approaches may be used for the reintroduction of cells into the host, including intravenous injection, intraperitoneal injection, or in situ injection into target tissue. Autologous and allogeneic cell transplantation may be used according to the invention.

The amount of rAAV particle or preparation or nucleic acid segment compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle or preparation or nucleic acid segment compositions, either over a relatively short, or a relatively prolonged period of time, as may be determined by the medical practitioner overseeing the administration of such compositions.

The composition may include rAAV particles or preparations or nucleic acid segments, either alone, or in combination with one or more additional active ingredients, which may be obtained from natural or recombinant sources or chemically synthesized. In some embodiments, rAAV particles or preparations are administered in combination, either in the same composition or administered as part of the same treatment regimen, with a proteasome inhibitor, such as Bortezomib, or hydroxyurea.

In other aspects, provided herein are methods of treatment comprising administration of a fusion protein as described herein to a subject in need thereof. In certain embodiments, methods of treatment comprise administering a pharmaceutical composition, rAAV particle, or nanoparticle as described herein.

To “treat” a disease as the term is used herein, means to reduce the frequency or severity of at least one sign or symptom of a disease or disorder experienced by a subject. The compositions described above are typically administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell.

In certain embodiments, provided herein are methods of treating a subject having or at risk of developing a disease, disorder, or condition comprising administering to the subject a fusion protein described herein. In particular embodiments, the fusion protein to be administered is a multimeric fusion protein. In other embodiments, the methods of treatment of a subject having or at risk of developing a disease, disorder, or condition comprise administering one or more pharmaceutical compositions, nanoparticles, or rAAV particles described herein.

In some embodiments, the subject has been diagnosed with a disease, disorder, or condition. In some embodiments, the subject has been diagnosed with an amyloid-related disease or disorder. In particular embodiments, the amyloid-related disease or disorder is Alzheimer's Disease or another neurodegenerative disorder. In other embodiments, the disease, disorder or condition is a cancer or an autoimmune disease.

Toxicity and efficacy of the compositions utilized in methods of the disclosure can be determined by standard pharmaceutical procedures, using either cells in culture or experimental animals to determine the LD₅₀ (the dose lethal to 50% of the population). The dose ratio between toxicity and efficacy the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED₅₀ with little or no toxicity. The dosage may vary within this range depending upon the dosage form employed and the route of administration utilized.

### Diagnostic Methods and Kits

In some aspects, the present disclosure provides diagnostic methods and kits for detecting an analyte. In some embodiments, methods are provided for determining a concentration of an analyte in a cell, tissue or organ, comprising: administering to the cell, tissue or organ of a subject one of the herein-described multimeric fusion proteins and measuring a concentration of the analyte; wherein the multimeric fusion protein has a binding affinity for the analyte. In other embodiments, methods for determining the concentration of the analyte or ligand in a cell, tissue or organ comprises administering a pharmaceutical composition or nanoparticle as described herein. In certain embodiments, the fusion protein of the disclosed diagnostic methods is capable of binding two or more analytes. These methods and kits may take advantage of any known method to measure or means for measuring binding affinity between the fusion protein and an analyte of interest, including equilibrium dialysis, equilibrium binding, gel filtration, ELISA, surface plasmon resonance, or spectroscopy (e.g., using a fluorescence assay). Exemplary conditions for evaluating binding affinity may be in HBS-P buffer (10 mM HEPES pH 7.4, 150 mM NaCl, 0.005% (v/v) Surfactant P20).

In some embodiments, of the disclosed methods, the cell is a mammalian cell derived or isolated from a mammalian subject. The mammalian subject may be diagnosed with or suffering from a disease or disorder, such as Alzheimer's Disease. In some embodiments, the disclosed methods may additionally comprise adjusting a treatment (e.g., a treatment regimen) of the mammalian subject in view of a result of a diagnostic assay (e.g., a concentration of the analyte).

In other aspects, the present disclosure provides kits for detecting an analyte comprising a purified multimeric fusion protein and vehicle for delivery that may comprise one or more pharmaceutically acceptable excipients. In certain embodiments, the kits comprise a nanoparticle comprising one or more multimeric fusion proteins. In certain embodiments, the fusion protein of the disclosed kits is capable of binding two or more analytes. The kits may comprise a means for isolating a mammalian cell from a mammalian subject and/or a means for isolating the analyte from the cell. Any of the disclosed kits may comprise a means for measuring binding affinity between the fusion protein and the analyst selected from equilibrium dialysis, equilibrium binding, gel filtration, ELISA, surface plasmon resonance, and spectroscopy.

In particular embodiments, methods and kits are provided for detecting the binding affinity of a fusion protein herein described to a ligand. In particular embodiments, these methods comprise incubating a multimeric fusion protein with the ligand; wherein each monomer of the fusion protein comprises a CCD comprising at least 10 G-X-Y repeats; wherein G is glycine, X is any amino acid, and Y is any amino acid, wherein at least 10 of the G-X-Y repeats are G-P-P or G-P-O, wherein at least 6 of the G-X-Y repeats are G-P-O, and wherein P is proline and O is hydroxyproline; and an antibody domain; wherein the hydroxyprolinated collagen-like domains of each one monomer interact with one another to form a multimeric protein that binds to a ligand with an avidity of at least 10⁷ M⁻¹; and detecting the binding of the multimeric protein to the ligand.

Recombinant AAV (rAAV) Particles and Genomes

Aspects of the disclosure relate to recombinant adeno-associated virus (rAAV) particles or preparations of such particles for delivery of one or more polynucleotides or vectors comprising a sequence encoding a heterologous peptide, into various tissues, organs, and/or cells. In some embodiments, the rAAV particle is delivered to a host cell as described herein.

The wild-type AAV genome is a single-stranded deoxyribonucleic acid (ssDNA), either positive- or negative-sensed. The genome comprises two inverted terminal repeats (ITRs), one at each end of the DNA strand, two open reading frames (ORFs): rep and cap between the ITRs, and an insert nucleic acid positioned between the ITRs and optionally comprising a transgene. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

Recombinant AAV (rAAV) particles may comprise a nucleic acid segment, which may comprise at a minimum: (a) one or more transgenes comprising a sequence encoding a heterologous peptide or an RNA of interest (e.g., a siRNA or microRNA) and (b) one or more regions comprising inverted terminal repeat (ITR) sequences (e.g., engineered ITR sequences) flanking the one or more heterologous nucleic acid regions (e.g., transgenes). In some embodiments, the nucleic acid segment is between 4 kb and 5 kb in size (e.g., 4.2 to 4.7 kb in size). Any nucleic acid segment described herein may be encapsidated by a viral capsid, such as an AAV2 capsid or another serotype (e.g., a serotype that is of the same serotype as the ITR sequences), which may comprises a modified capsid protein as described herein. In particular embodiments, the capsid is an AAV2/8 or AAV2/1 serotype.

In some embodiments, the nucleic acid segment is circular. In some embodiments, the nucleic acid segment is single-stranded. In some embodiments, the nucleic acid segment is double-stranded. In some embodiments, a double-stranded nucleic acid segment may be, for example, a self-complimentary vector that contains a region of the nucleic acid segment that is complementary to another region of the nucleic acid segment, initiating the formation of the double-strandedness of the nucleic acid segment.

Accordingly, in some embodiments, an rAAV particle or rAAV preparation containing such particles comprises a viral capsid and a nucleic acid segment as described herein, which is encapsidated by the viral capsid. In some embodiments, the insert nucleic acid of the nucleic acid segment comprises (1) one or more transgenes comprising a sequence encoding a heterologous peptide, (2) one or more nucleic acid regions comprising a sequence that facilitates expression of the transgene (e.g., a promoter), and (3) one or more nucleic acid regions comprising a sequence that facilitate integration of the transgene (optionally with the one or more nucleic acid regions comprising a sequence that facilitates expression) into the genome of the subject. In certain embodiments, the promoter of the insert nucleic acid comprises a sequence that has at least 90%, at least 95%, or at least 99% identity to a chicken β-actin (CBA) promoter.

In some embodiments, the polynucleotides and vectors described herein comprise ITR sequences. In some embodiments, the coding sequence and associated promoter are flanked by rAAV ITR sequences. The ITR sequences of a polynucleotide described herein can be derived from any AAV serotype (e.g., 1, 2, 3, 4, 5, 6, 7, 8, 9, or 10) or can be derived from more than one serotype. In some embodiments, the ITR sequences are derived from AAV2 or AAV6. In some embodiments, the ITR sequences of the first serotype are derived from AAV3, AAV2 or AAV6. In other embodiments, the ITR sequences of the first serotype are derived from AAV1, AAV5, AAV8, AAV9 or AAV10. In some embodiments, the ITR sequences are the same serotype as the capsid (e.g., AAV3 ITR sequences and AAV3 capsid, etc.).

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, et al. Proc Natl Acad Sci USA. 1996; 93(24):14082-7; and Curtis A. Machida, Methods in Molecular Medicine™ Viral Vectors for Gene Therapy Methods and Protocols. 10.1385/1-59259-304-6:201 Humana Press Inc. 2003: Chapter 10, Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the nucleic acid segment comprises a pTR-UF-11 plasmid backbone, which is a plasmid that contains AAV2 ITRs. This plasmid is commercially available from the American Type Culture Collection (ATCC MBA-331).

The rAAV particle comprising a nucleic acid segment (in any form contemplated herein) may be delivered in the form of a composition, such as a composition comprising the active ingredient, such as the rAAV particle, nucleic acid segment (in any form contemplated herein), and a therapeutically or pharmaceutically acceptable carrier. The rAAV particles or nucleic acid segment may be prepared in a variety of compositions, and may also be formulated in appropriate pharmaceutical vehicles for administration to human or animal subjects.

Other aspects of the disclosure are directed to methods that involve contacting cells with an rAAV preparation produced by a method described herein. The contacting may be, e.g., ex vivo or in vivo by administering the rAAV preparation to a subject. The rAAV particle or preparation may be delivered in the form of a composition, such as a composition comprising the active ingredient, such as a rAAV particle or preparation described herein, and a therapeutically or pharmaceutically acceptable excipient. The rAAV particles or preparations may be prepared in a variety of compositions, and may also be formulated in appropriate pharmaceutical vehicles for administration to human or animal subjects.

To achieve appropriate or enhanced expression levels of the heterologous peptide, any of a number of heterologous promoters suitable for use in the selected host cell may be employed. The promoter may be, for example, a constitutive promoter, tissue-specific promoter, inducible promoter, or a synthetic promoter.

Inducible promoters and/or regulatory elements may also be contemplated for achieving appropriate expression levels of the heterologous peptide. Non-limiting examples of suitable inducible promoters include the CBA promoter and those promoters from genes such as cytochrome P450 genes, heat shock protein genes, metallothionein genes, and hormone-inducible genes, such as the estrogen gene promoter.

Tissue-specific promoters and/or regulatory elements are also contemplated herein. In certain embodiments, the heterologous promoters of the disclosed fusion proteins are active in brain cells, such as human brain cells (e.g. human neurons and glia). Exemplary promoters active in human brain cells include, but are not limited to, the human synapsin 1 gene promoter (SYN), the hybrid CMV enhancer/chicken β-actin (CAG) promoter, Glial Fibrillary Acidic protein (GFAP) promoter, Microtubule-associated protein 2 (MAP2) promoter, and the platelet-derived growth factor-β chain promoter (1500 bp). These promoters are described in, e.g., Kugler et al. Virology, 311(1):89-95 (2003) and Morelli et al. J Gen Virol., 80(Pt 3):571-83 (1999), which are incorporated herein by reference. Non-limiting examples of such promoters that may be used include species-specific promoters, such as human-specific promoters.

Synthetic promoters are also contemplated herein. A synthetic promoter may comprise, for example, regions of known promoters, regulatory elements, transcription factor binding sites, enhancer elements, repressor elements, and the like.

The rAAV particle or particle within an rAAV preparation may be of any AAV serotype, including any derivative or pseudotype (e.g., 1, 2, 3, 4, 5, 6, 7, 8, 9, 10, 2/1, 2/5, 2/6, 2/8, 2/9, 3/1, 3/5, 3/8, or 3/9). As used herein, the serotype of an rAAV viral vector (e.g., an rAAV particle) refers to the serotype of the capsid proteins of the recombinant virus. In some embodiments, the rAAV particle is not AAV2. In some embodiments, the rAAV particle is not AAV8. Non-limiting examples of derivatives and pseudotypes include rAAV2/1, rAAV2/5, rAAV2/6, rAAV2/8, rAAV2/9, AAV2-AAV3 hybrid, AAVrh.10, AAVrh.74, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2(Y→F), AAV8 (Y733F), AAV2.15, AAV2.4, AAVM41, AAV-DJ and AAVr3.45. These AAV serotypes and derivatives/pseudotypes, and methods of producing such derivatives/pseudotypes are known in the art (see, e.g., Mol Ther. 2012 April; 20(4):699-708). The AAV vector toolkit: poised at the clinical crossroads. Asokan A1, Schaffer D V, Samulski R J.). In some embodiments, the rAAV particle is a pseudotyped rAAV particle, which comprises (a) a nucleic acid segment comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Additional serotypes of the rAAV capsids disclosed herein include capsids include AAV2, AAV6 and capsids derived from AAV2 and AAV6. In addition, such capsids include AAV7m8, AAV2/2-MAX, AAVSHh10Y, AAV3, AAV3b, AAVLK03, AAV7BP2, AAV1(E531K), AAV6(D532N), AAV6-3pmut and AAV2G9.

The AAV-DJ capsid is described in Grimm et al., J. Virol., 2008, 5887-5911 and Katada et al., (2019) Evaluation of AAV-DJ vector for retinal gene therapy, PeerJ 7:e6317 each of which is herein incorporated by reference. The AAV-DJ comprises the insertion of 7 amino acids into the HSPG binding domain of the AAV2 capsid and has high expression efficiency in Muller cells following intravitreal injection. The AAV7m8 capsid, which is closely related to AAV-DJ, is described in Dalkara et al. Sci Transl Med. 2013; 5(189):189ra76, herein incorporated by reference.

The AAV2/2-MAX capsid is described in Reid, Ertel & Lipinski, Improvement of Photoreceptor Targeting via Intravitreal Delivery in Mouse and Human Retina Using Combinatory rAAV2 Capsid Mutant Vectors, Invest. Ophthalmol Vis Sci. 2017; 58:6429-6439, herein incorporated by reference. The AAV2/2-MAX capsid comprises five point mutations, Y272F, Y444F, Y500F, Y730F, T491V. The AAV1(E531K) capsid is described in Boye et al., Impact of Heparan Sulfate Binding on Transduction of Retina by Recombinant Adeno-Associated Virus Vectors, J. Virol. 90:4215-4231 (2016), herein incorporated by reference.

The AAVSHh10 and AAV6(D532N) capsids, both derivatives of AAV6, are described in Klimczak et al. (2009) A Novel Adeno-Associated Viral Variant for Efficient and Selective Intravitreal Transduction of Rat Muller Cells. PLoS ONE 4(10): e746, herein incorporated by reference. The AAV6-3pmut (also known as AAV6(TM6) and AAV6(Y705+Y731F+T492V)) capsid is described in Rosario et al., Microglia-specific targeting by novel capsid-modified AAV6 vectors, Mol Ther Methods Clin Dev. 2016; 13; 3:16026 and International Patent Publication No. 2016/126857, each of which are herein incorporated by reference.

Additional capsids suitable for use in the disclosed rAAV particles include the following: capsids comprising non-native amino acid substitutions at amino acid residues of a wild-type AAV2 capsid, wherein the non-native amino acid substitutions comprise one or more of Y272F, Y444F, T491V, Y500F, Y700F, Y704F and Y730F; capsids comprising non-native amino acid substitutions at amino acid residues of a wild-type AAV6 capsid, wherein the non-native amino acid substitutions comprise one or more of Y445F, Y705F, Y731F, T492V and S663V.

### Production Methods

Methods of producing rAAV particles and nucleic acid segments are described herein. Other methods are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Nos. US 2007/0015238 and US 2012/0322861, which are incorporated herein by reference; and plasmids and kits available from ATCC and Cell Biolabs, Inc.). For example, a plasmid containing the nucleic acid segment may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the one or more helper plasmids include a first helper plasmid comprising a rep gene and a cap gene and a second helper plasmid comprising a Ela gene, a E1b gene, a E4 gene, a E2a gene, and a VA gene. In some embodiments, the rep gene is a rep gene derived from AAV3, AAV5, or AAV6 and the cap gene is derived from AAV2, AAV3, AAV5, or AAV6 and may include modifications to the gene in order to produce the modified capsid protein described herein. In some embodiments, the rep gene is a rep gene derived from AAV1 or AAV2 and the cap gene is derived from AAV1 or AAV2 and may include modifications to the gene in order to produce the modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes for a first serotype (e.g., AAV3, AAV5, and AAV6), cap genes (which may or may not be of the first serotype) and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO₄-mediated transfection, lipids or polymeric molecules such as Polyethylenimine (PEI) with the helper plasmid(s) and a plasmid containing a nucleic acid segment described herein. The HEK293 cells are then incubated for at least 60 hours to allow for rAAV particle production. Alternatively, in another example Sf9-based producer stable cell lines are infected with a single recombinant baculovirus containing the nucleic acid segment. As a further alternative, in another example HEK293 or BHK cell lines are infected with a HSV containing the nucleic acid segment and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

### Host Cells

The disclosure also contemplates host cells that comprise at least one of the disclosed rAAV particles or nucleic acid segments described herein. Such host cells include mammalian host cells, with human host cells being preferred, and may be either isolated, in cell or tissue culture. In the case of genetically modified animal models (e.g., a mouse), the transformed host cells may be comprised within the body of a non-human animal itself.

The disclosure also contemplates host cells that comprise at least one of the disclosed rAAV particles or nucleic acid segments. Such host cells include mammalian host cells, with human host cells being preferred, and may be either isolated, in cell or tissue culture. In the case of genetically modified animal models (e.g., a mouse), the transformed host cells may be comprised within the body of a non-human animal itself. In some embodiments, the host cell is a cancer cell. In some embodiments, the host cell is a liver cell, such as a liver cancer cell. In certain embodiments, the host cells are HEK293T cells or HeLa cells.

In some embodiments, a host cell as described herein is derived from a subject as described herein. Host cells may be derived using any method known in the art, e.g., by isolating cells from a fluid or tissue of the subject. In some embodiments, the host cells are cultured. Methods for isolating and culturing cells are well known in the art.

Delivery Methods Other than rAAV

In some aspects, the present disclosure provides methods of delivering one or more purified proteins, such as the CCD fusion proteins described herein, or one or more polynucleotides or vectors encoding these fusion proteins, to a subject or a host cell or tissue. In some embodiments, the method of delivery provided comprises nanoparticle delivery, nucleofection, microinjection, biolistics, virosomes, liposomes, immunoliposomes, polycation or cationic lipid conjugates, naked DNA, artificial virions, and agent-enhanced uptake of DNA.

Exemplary methods of delivery of polynucleotides and expression vectors include cationic polymer:DNA complexes (e.g. using polyethylenimine (PEI)), lipofection, nucleofection, electoporation, stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 1991/17424 and WO 1991/16024. Delivery may be to cells (e.g. in vitro or ex vivo administration) or target tissues (e.g. in vivo administration).

Exemplary methods of delivery of purified fusion proteins include nanoparticle and lipid particles, such as cationic lipid:peptide conjugates. Exemplary nanoparticles may be protein-based or polymer-based and are known in the art.

### Subjects

Aspects of the disclosure relate to methods and preparations for use with a subject, such as human or non-human primate subjects, a host cell in situ in a subject, or a host cell derived from a subject. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease that may be treated with gene therapy. In some embodiments, the subject has or is suspected of having a hemoglobinopathy. A hemoglobinopathy is a disease characterized by one or more mutation(s) in the genome that results in abnormal structure of one or more of the globin chains of the hemoglobin molecule. Exemplary hemoglobinopathies include hemolytic anemia, sickle cell disease, and thalassemia. Sickle cell disease is characterized by the presence of abnormal, sickle-chalped hemoglobins, which can result in severe infections, severe pain, stroke, and an increased risk of death. Subjects having sickle cell disease can be identified, e.g., using one or more of a complete blood count, a blood film, hemoglobin electrophoresis, and genetic testing. Thalassemias are a group of autosomal recessive diseases characterized by a reduction in the amount of hemoglobin produced. Symptoms include iron overload, infection, bone deformities, enlarged spleen, and cardiac disease. The subgroups of thalassemias include alpha-thalassemia, beta-thalassemia, and delta thalassemia. Subjects having a thalassemia may be identified, e.g., using one or more of complete blood count, hemoglobin electrophoresis, Fe Binding Capacity, urine urobilin and urobilogen, peripheral blood smear, hematocrit, and genetic testing.

In some embodiments, the subject has or is suspected of having a disease that may be treated with gene therapy. In some embodiments, the subject has or is suspected of having a proliferative disease, such as cancer. The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is liver cancer. Exemplary liver cancers include, but are not limited to, hepatocellular carcinoma (HCC), cholangiocarcinoma, angiosarcoma, and hepatoblastoma. Subject having cancer can be identified by the skilled medical practitioner, e.g., using methods known in the art including biopsy, cytology, histology, endoscopy, X-ray, Magnetic Resonance Imaging (MRI), ultrasound, CAT scan (computerized axial tomography), genetic testing, and tests for detection of tumor antigens in the blood or urine.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: CCD Domains Dramatically Increase Secretion of Fusion Protein

To identify an optimal C1QTNF family member for multimeric fusion experiments, CCD domains derived from two isotypes of the C1QTNF protein, i.e., C1QTNF3 and C1QTNF5 was tested. CCD from those two proteins were fused to soluble Lag3. Results show both CCD increased secretion of sLag3 (middle panel), but CCD from C1QTNF3 had much higher potency to increase multimerization (right panel) of sLag3. Results are shown in FIG. 22.

Many decoy peptides comprise truncated polypeptides and are often attacked by peptidases in vivo; thus they are often subject to low yields during use in a mammalian overexpression system. Plasmids encoding various CCD fusions with decoy heterologous peptides comprising extracellular (soluble) and intracellular peptides were designed for improved overexpression in the cell to overcome this deficiency. These peptides include soluble Lag3, soluble PD-1, CTLA4, NOTCH1, DLL3 and Aβ9 scFv (see FIG. 2). These plasmids further included FLAG tags for facilitation of subsequent Western blotting experiments.

To evaluate the effect of fusion with a CCD domain on the overexpression and secretion levels of these peptides, plasmids encoding the CCD fusion proteins and plasmids encoding the heterologous peptides alone were encapsidated into an rAAV particle and then transiently transfected into HEK 293T cells using polyethylenimine (PEI) transfection. PEI is a well-known cationic polymer.

16 hours after transfection, cell media was replaced with fresh Dulbecco's Modified Eagle Medium (DMEM) supplied with 2% fetal bovine serum (FBS). Cells and media were harvested after 24 hours. Cells were lysed with ice cold PBS (1% Triton-100). Cell debris was separated from the media by 18000 g centrifugation for 10 minutes. SDS-PAGE was performed in the absence of a reducing agent on the cell lysates and media.

Western blots were developed and imaged with anti-FLAG M2 antibody as shown in FIG. 2 (CCD fusions are designated as “[peptide]_Collagen”). Both overexpression levels (as seen in the cell lysate blot) and secretion levels (media blot) were dramatically increased in the CCD fusion proteins relative to peptides alone. In some cases, expression of the peptide improved from undetectable levels (in the lysate and/or media blots) when expressed without the CCD to a very high level when expressed with the CCD (e.g. sPD-1, DLL3 and NOTCH1).

Two possible causes for the increase in expression and secretion levels are hypothesized. Foremost, multimerization of CCD fusion proteins in vivo may protect intracellular heterologous peptides from attack by endopeptidases. And the CCD domain may protect extracellular heterologous peptides from proteolytic attack by exopeptidases, such as aminopeptidases and carboxypeptidases, whose mechanisms of action are dependent on which terminus of the heterologous peptide the CCD was positioned. For instance, sDLL3 was not observed in the cell lysate blot but sDLL3-CCD was clearly detected in the same condition. Although secretion level for this CCD fusion protein was relatively low, it was determined that sDLL3-CCD surprisingly localized at the cell surface. Cell surface localization will not likely effect the function of this peptide, as its ligands are mostly membrane proteins.

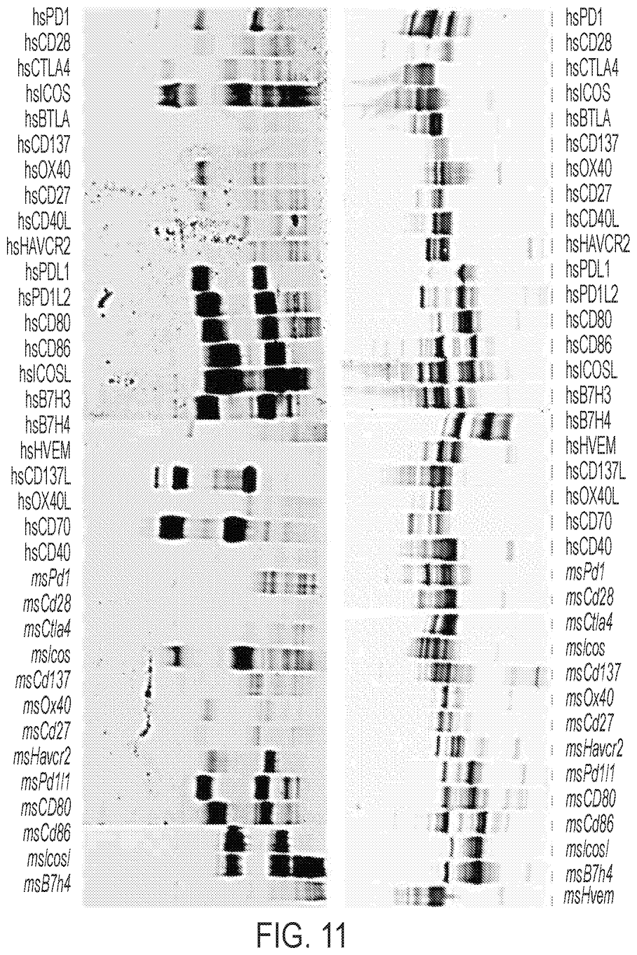

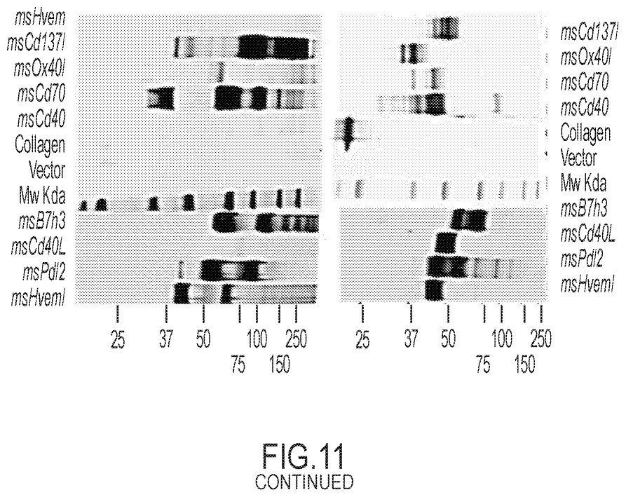

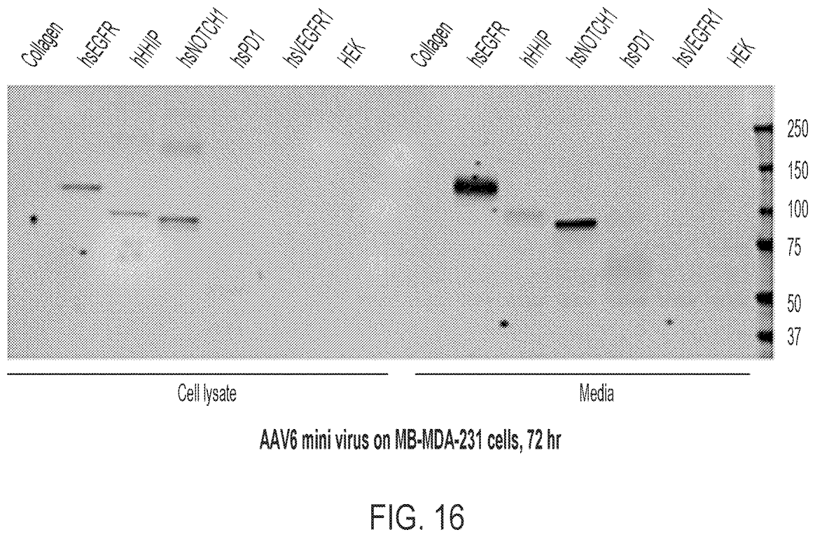

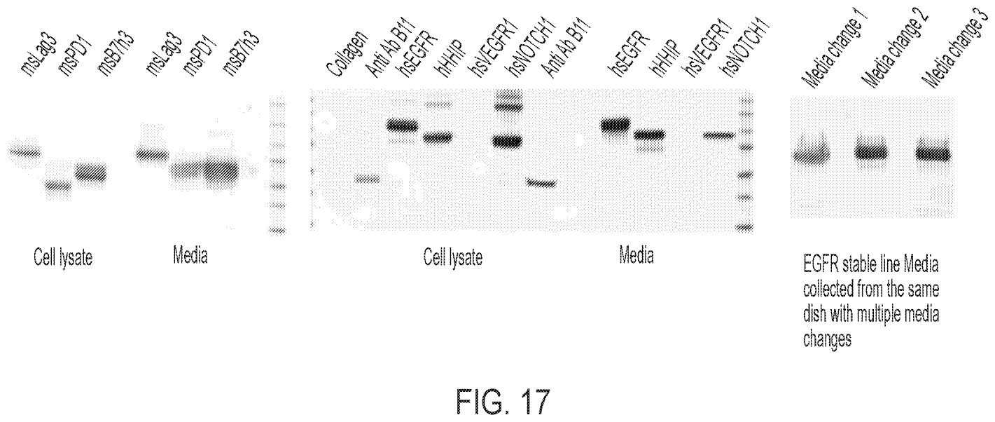

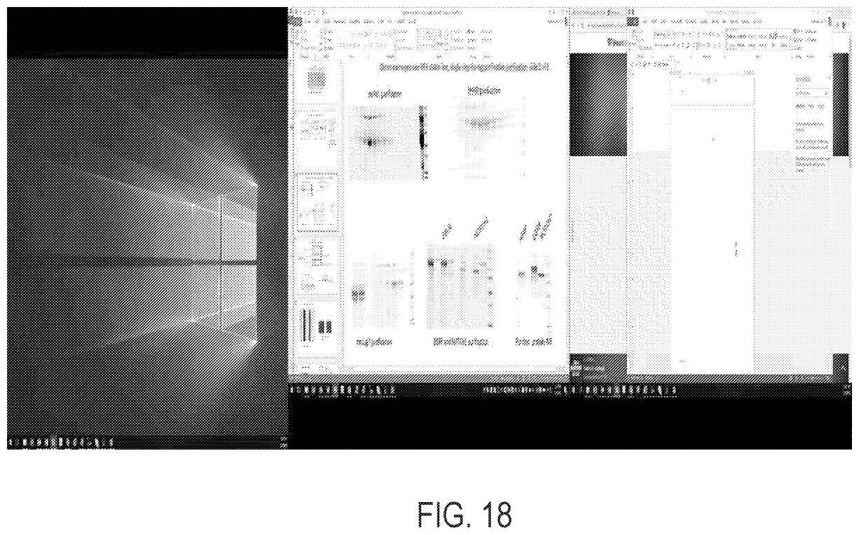

The expression and secretion levels of various additional CCD fusion proteins were analyzed after a) rAAV administration to mouse and human cells or b) incubation of cells with purified proteins. Western blot results in cell lysates and media are shown in FIGS. 11-18.

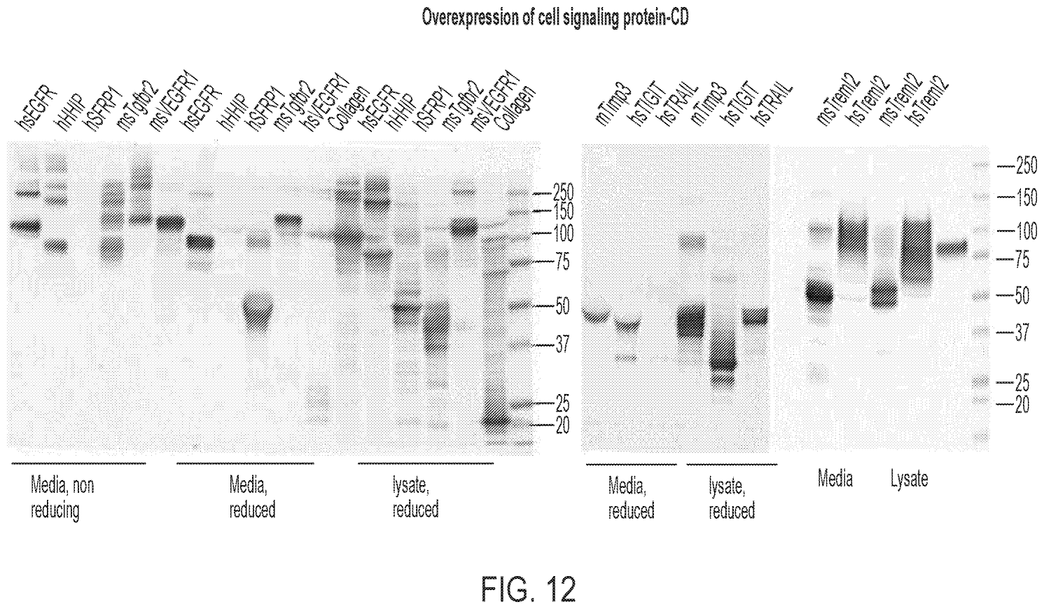

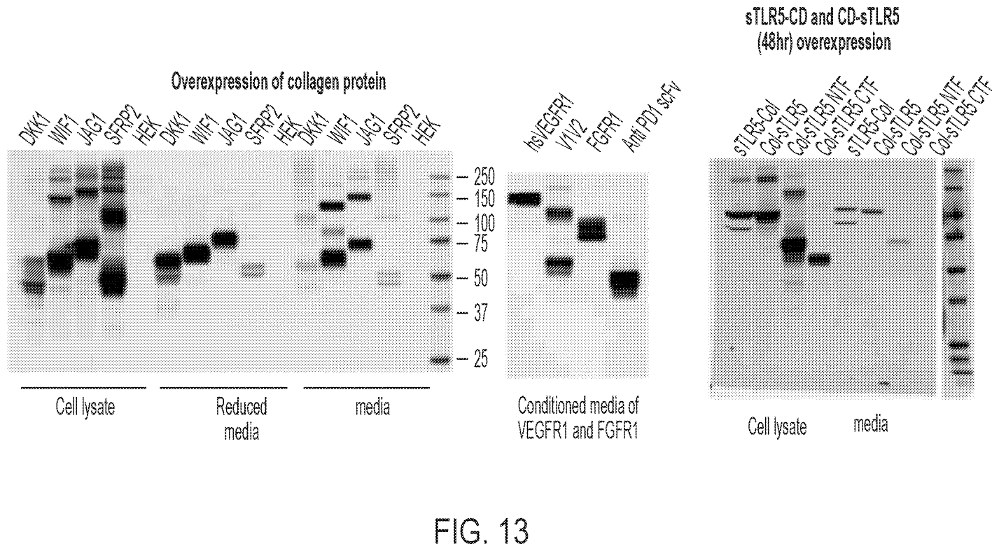

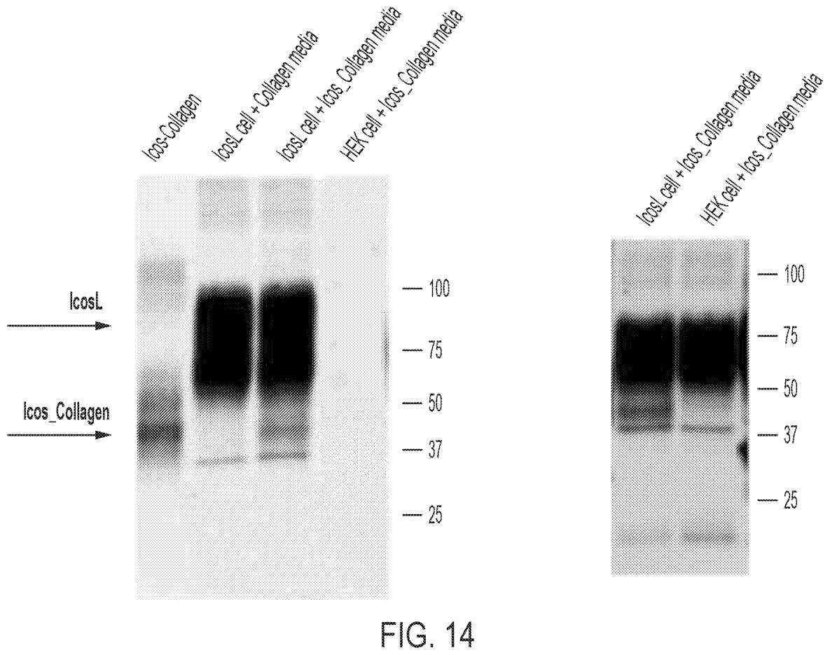

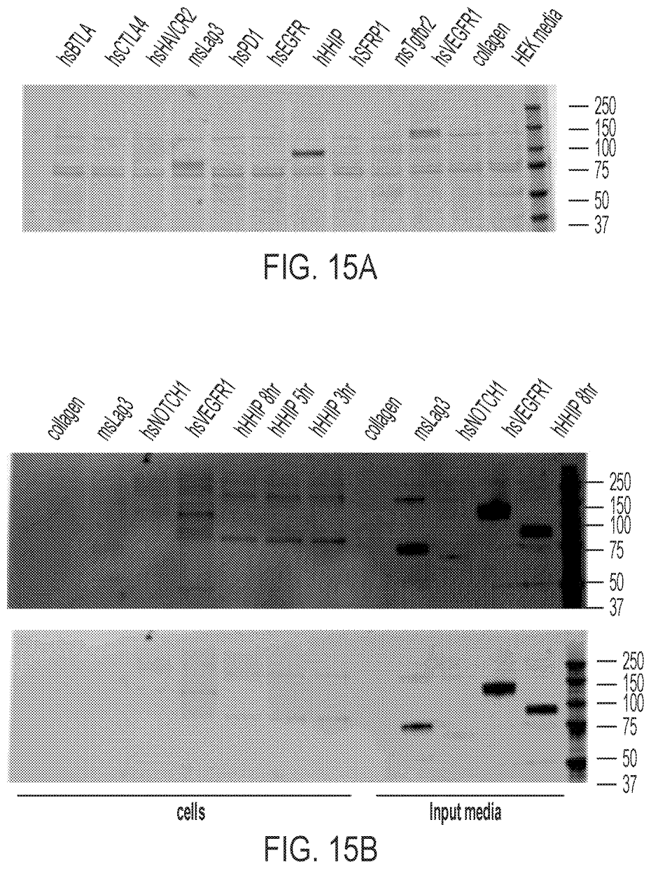

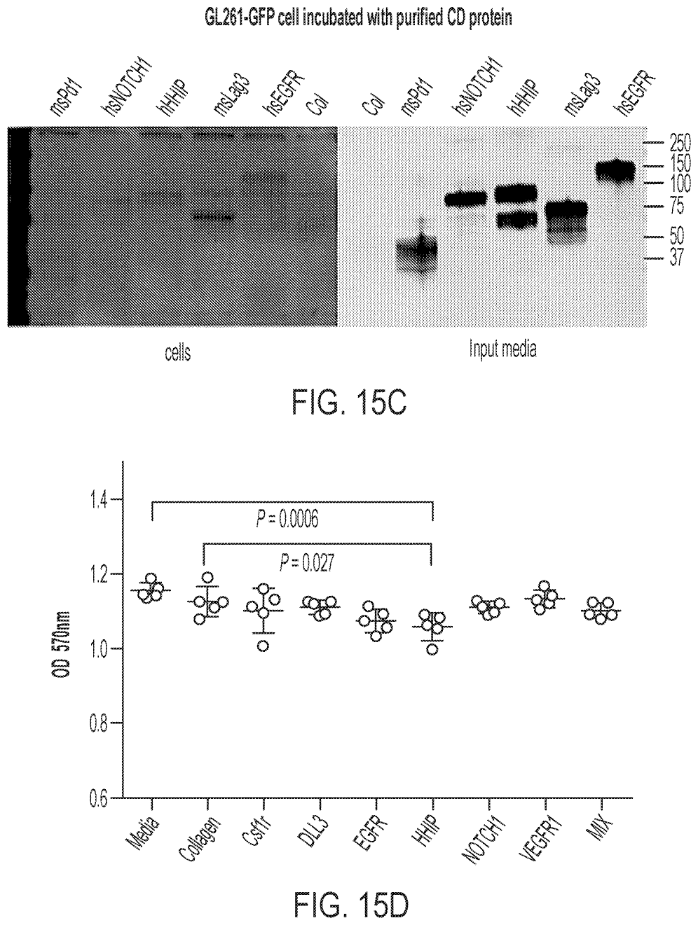

As an example, as shown in Western blots of FIG. 13, overexpression and secretion of various fusion proteins of CCD and soluble Toll-like receptors in both human and mouse tissues was achieved following administration of rAAV vectors expressing an sTLR-CCD fusion construct. The blots compare overexpression and secretion when the CCD is positioned at the N-terminus or C-terminus of sTLR5, where cells were harvested 48 hours post-transfection. And the Western blots of FIG. 14 indicate that rAAV-mediated administration to HEK 293T cells, in the presence or absence of media containing Icos ligands, induced overexpression of Icos-CCD fusion proteins. FIGS. 11 and 12 show Western blots generated after similar experiments with rAAV vectors encoding CCD conjugates of human-derived and mouse-derived immune checkpoint proteins, and cell signaling heterologous peptides, respectively. And as shown in the Western blots of FIGS. 15A-15C, CCD fusion protein constructs comprising a sLag3, a NOTCH1, a HHIP, a VEGFR1, a Tgfbr2, a PD-1, a HAVCR2, a CTLA4, a BTLA, a SFRP1 or an EGFR peptide bind to the cell surface after incubation of purified fusion proteins with HEK293T and mouse glioma GL261 cells, followed by lysis and separation.

### Example 2: CCD Fusion Proteins are Multimeric

An important parameter to evaluate a decoy protein is its ligand binding affinity. Avidity measures the total binding strength of a decoy protein or antibody. One effective method to enhance the avidity of a decoy or antibody is through multivalence engineering. As mentioned above, native collagen usually forms trimers through association of glycine repeat domains. Multimerization of CCD fusion proteins may enhance the avidities of the heterologous peptides.

To test the hypothesis that a multimerization (e.g. into trimers) of CCD fusion proteins in vivo may protect intracellular heterologous peptides from attack by endopeptidases, an rAAV nucleic acid vector encoding sLag3-CCD_FLAG was overexpressed in HEK 293T cells by PEI transfection as described above. Conditioned (or spent) cell media was collected. (The conditioned media contains the secreted CCD fusion proteins.) Following concentration to a suitable volume, media were loaded onto a PBS pre-equilibrated size exclusion column (Superose® 6 10/300 GL, GE, USA) connected to ÄKTA® Prime FPLC system (GE). 0.3 ml eluates were collected for each fraction. SDS-PAGE was performed in the absence of a reducing agent.

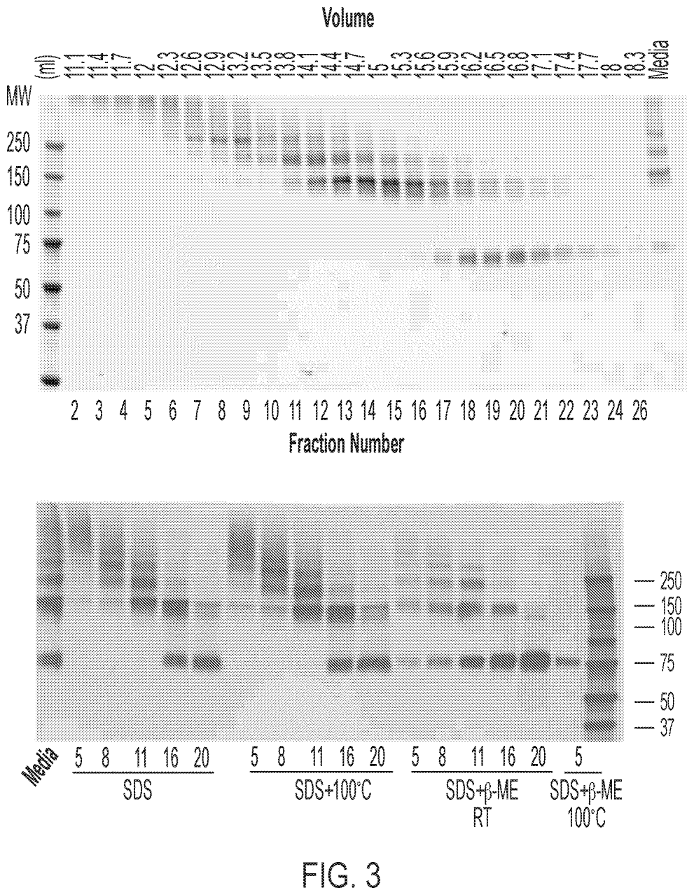

Western blots were developed and imaged with anti-FLAG M2 antibody as shown in FIG. 2. As seen in left panel of FIG. 3, a greater concentration of multimeric CCD fusion proteins were expressed than monomeric fusion proteins. Hexamers of fusion proteins (bands at top edge of elution fractions 2-7), tetramers (bands corresponding to about 250 kDa in fractions 6-13), trimers (bands between 150 and 250 kDa in fractions 9-17), dimers (bands between 100 and 150 kDa in fractions 11-20), and monomers (bands between 50 and 750 kDa in fractions 9-17) were sequentially eluted. A schematic illustrating the probable structure of CCD fusion trimers is shown in FIG. 1.

Several 0.3 ml elution fractions from this experiment (fraction numbers 5, 8, 11, 16 and 20) were subjected to a variety of temperature and reduction conditions: storage at room temperature (RT) or 100° C.; and in the presence and absence of reducing agent β-mercaptoethanol (β-ME). β-mercaptoethanol cleaves disulfide bonds. Fractions were analyzed by SDS-PAGE. A larger range of monomeric to hexameric proteins can be seen in FIG. 3, right panel than that of FIG. 3, left panel. Oligomers of sLag3-CCD were heat resistant. Treatment with reducing agent and heat led to dissociation of the oligomers.

These results suggest that sLag3-CCD forms multimers efficiently, and that multimers are stabilized through disulfide bonds.

### Example 3: CCD Fusion Proteins have Long Stability and can be Expressed Efficiently in Mouse

The stability of a sB7H3-CCD fusion protein monomer was tested after storage for up to one month at room temperature and at 4° C. sB7H3-CCD was expressed in cells by delivery of an rAAV vector encoding this protein. Cells were lysed and protein was isolated and divided into aliquots. Protein purified from conditioned media was used for SDS-PAGE and Coomassie staining to visualize protein stability.

Protein samples were aliquots at −80° C. prior to stability analysis, and then suspended in PBS with 150 mM NaCl. SDS-PAGE and Western blot analyses were compared before and after storage, to determine if any protein bands appeared that indicated the presence of degradants.

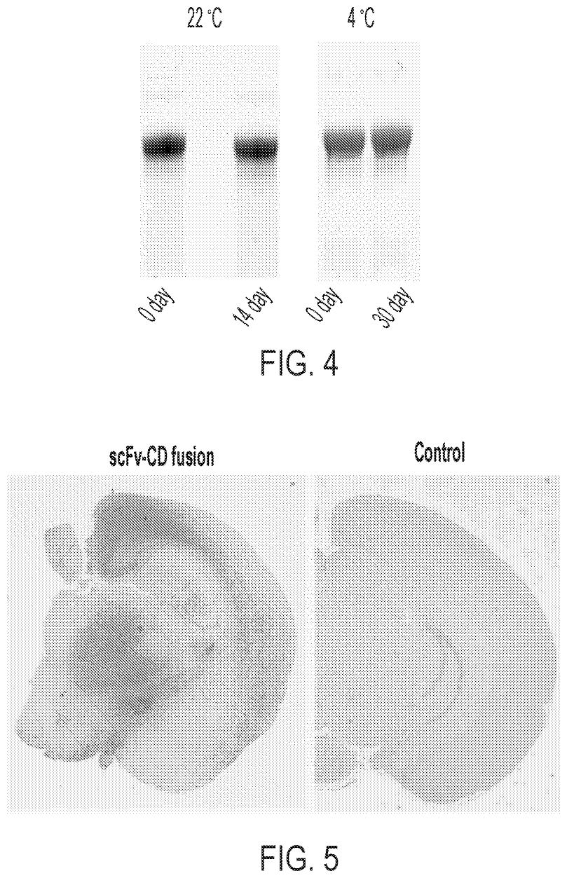

As shown in FIG. 4, no significant change in blot image was observed after two weeks of storage at room temperature or after 30 days of storage at 4° C., indicating an absence of degradants. This results suggests that the CCD fusion proteins may have a long shelf life.

To test if exemplary CCD fusion proteins could be efficiently delivered and expressed in vivo, rAAV particles comprising a vector encoding anti-amyloid beta 5 antibody fragment (Aβ5 scFv) CCD fusion proteins were synthesized. rAAV particles were injected into mice. Two weeks after the injection, mice were sacrificed, and the scFv-CCD protein was detected with immune staining of mouse brain.

Aβ5 scFv-CCD fusion proteins were overexpressed (darker areas) at a very high level as shown in FIG. 5. The results suggest that CCD fusion proteins can be efficiently overexpressed in animal models.

### Example 4: CCD Fusion Proteins are Biologically Active

The ultimate goal is to generate active heterologous peptides capable of overexpression after gene delivery, e.g. through rAAV-mediated gene delivery. The CCD fusion strategy increased the expression and secretion level of the target protein, but it was not yet understood whether the CCD domain may disturb the structure and activity of the heterologous peptide. Thus, an FGFR1-CCD fusion protein was constructed, expressed in cells and then isolated and purified. Binding affinity of this protein was tested directly in in vitro binding and indirectly by a cell metabolic activity assay.

Using a surface plasmon resonance (SPR) technique, the binding kinetics of FGFR1 was measured with two ligands, basic FGF and acidic FGF. A null FGFR1 ligand, vascular endothelial growth factor A (VEGFA), was used as a control. In SPR experiments, the binding of a molecule in the soluble phase (e.g. the peptide of the fusion protein) to a ligand molecule immobilized on a sensor surface is directly measured as a reflected light signal (SPR signal) in a biosensor device. The sensor device may contain an LED-emitting near infrared light, a glass prism fixed to a sensor microchip, and a position-sensitive diode array detector. Binding events cause changes in the refractive index at the surface layer, which are detected as changes in the SPR signal. Specifically, a signal arises when incident LED light is totally internally reflected from the metal-coated interface between two media of different refractive indices (the glass prism and solution). When the incident light is focused on the surface in a wedge, the drop in intensity at the resonance angle appears as a “shadow” in the reflected light wedge, which is detected by the detector. When an interaction between the immobilized ligand and peptide in solution occurs, the “shadow” is shifted on the detector, i.e., the resonance angle changes.

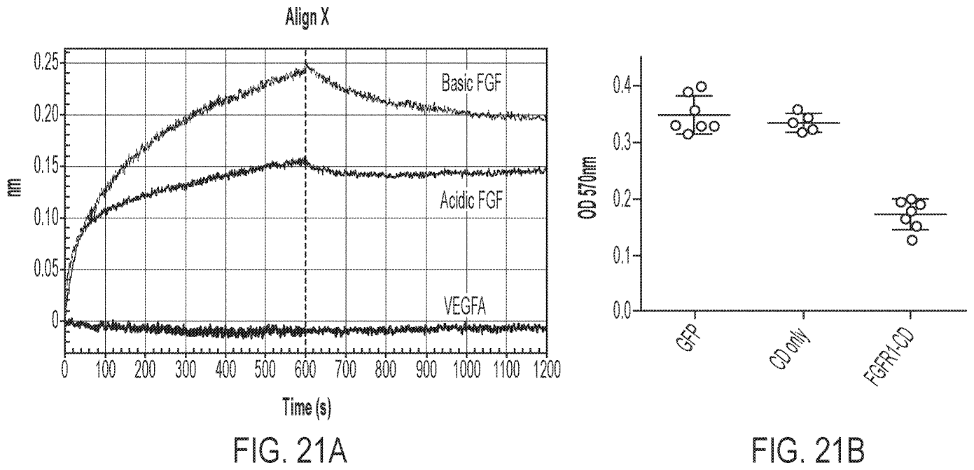

The sensorgram from the SPR experiment with FGFR1-CCD is shown in FIG. 21A. This sensorgram consists of two phases: (Left) In the association phase, the FGFR1-CCD was injected over immobilized ligands on the surface. With increasing interaction of FGFR1-CCD and ligands, an increasing SPR response is detected [displayed in response units (RU)]. Maximal binding is indicated at the “AlignX” time. (Right) After 600 seconds, injection of FGFR1-CCD into the biosensor was stopped and the system was switched back to buffer in a dissociation phase. Binding kinetics, including the value of K_{D}, the dissociation constant, can be calculated from the sensorgram.

The sensorgram shows that FGFR1-CCD fusion proteins efficiently captured both basic FGF and acidic FGF. The K_{D} of FGFR1-CCD and acidic FGF was calculated to be less than 1.0×10⁻¹² and was comparable to native FGFR1. This result suggests that fusion to a CCD domain did not affect the binding ability of FGFR1 significantly.

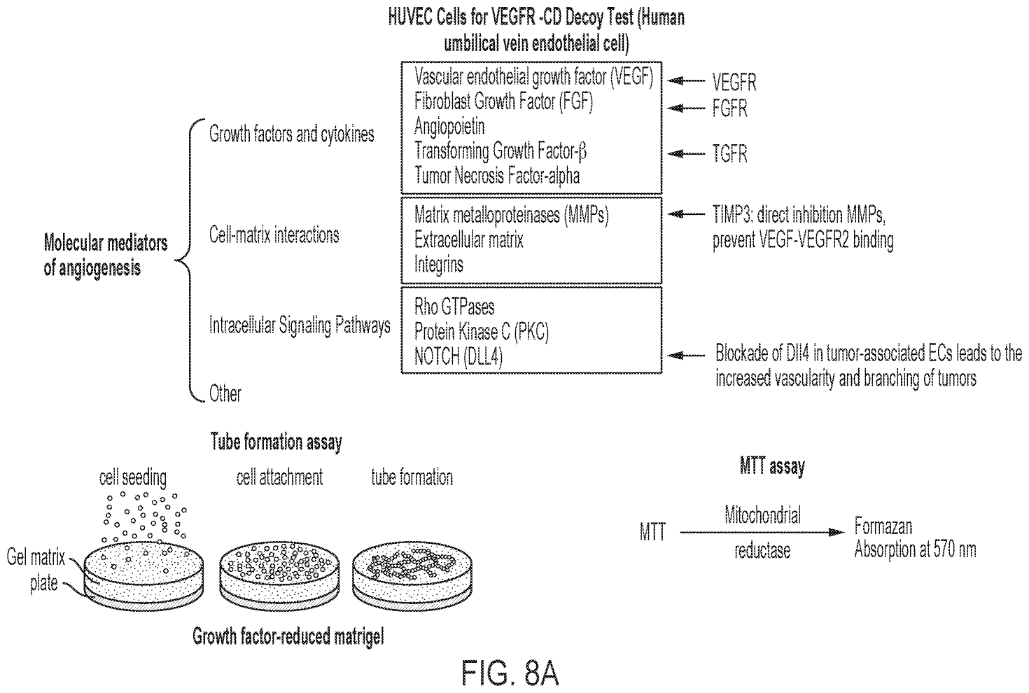

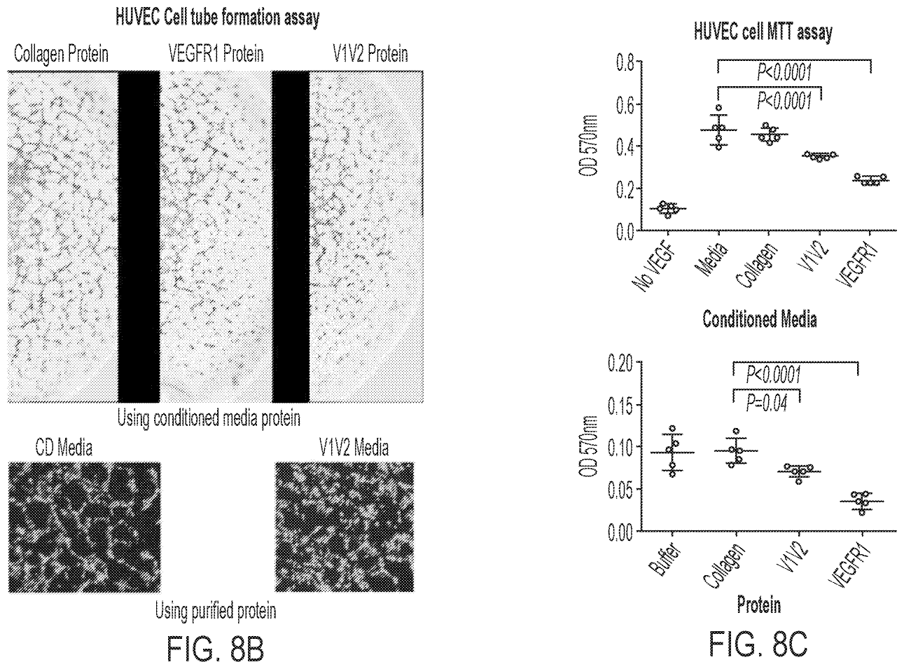

Next it was tested whether FGFR1-CCD could act as a functional decoy receptor and inhibit cell growth in vivo. A human umbilical vein endothelial cell line (HUVEC) was chosen for this experiment. An MTT assay was performed to assess HUVEC cell metabolic activity. In an MTT assay, an enzymatic reduction of 3-[4,5-dimethylthiazole-2-yl]-2,5-diphenyltetrazolium bromide (MTT) to MTT-formazan is catalyzed by mitochondrial succinate dehydrogenase (see FIG. 8A). Hence, the MTT assay is dependent on mitochondrial respiration and indirectly serves to assess the cellular energy capacity of a cell. The MTT assay is a colorimetric reaction that can easily be measured from cell monolayers that have been plated in 35 mm dishes or multiwell plates.

Conditioned media or purified FGFR1-CCD were added to HUVEC cell cultures and incubated for 48 hours. HUVEC cell cultures were incubated for 2 h in culture medium or in in a Krebs-Hensleit-HEPES buffer (115 mM NaCl, 5 mM KCl, 1 mM KH₂PO₄, 1.2 mM MgSO₄, 2 mM CaCl₂), and 25 mM HEPES at pH 7.4) containing 0.5 mg ml⁻¹ MTT. After 2 hours, the incubation buffer was removed and the blue MTT-formazan product was extracted with acidified isopropyl alcohol (0.04 N HCl). After 30 minutes of extraction at room temperature, the absorbance of the formazan solution was read spectrophotometrically at 570 nm.

As seen FIGS. 21B and 8C, FGFR1-CCD substantially inhibited cell growth up to 50%. The results suggest that FGFR1-CCD is biologically active as a decoy receptor, successfully diverting FGF ligands away from binding to native FGFR receptors within the cell. These results were substantiated in a HUVEC cell tube formation assay, in which FGFR1-CCD likewise inhibited growth of HUVEC cell tubes in growth factor-reduced matrigel (see FIG. 8B).

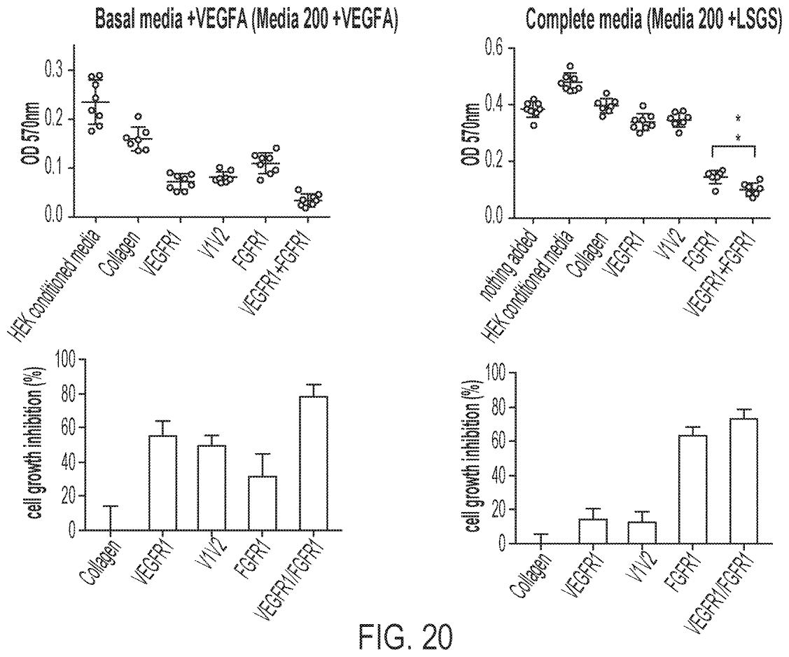

MTT assays were also performed in HUVEC cells that were incubated with various purified CCD fusion proteins, as shown in FIG. 20. Cells incubated with 100 μl of 50:50 basal media 200 or 100% basal media with 50 μg/ml purified human VEGFR1-CCD; a fusion protein comprising human VEGFR1 domain 2 and human VEGFR2 domain 3 (“V1V2-CCD”); or VEGFR1-FGFR1-CCD. VEGFA (left graph) or low serum growth supplement (right graph) was added to cell media at a final concentration of 2 ng/ml. Plots of cell viability and inhibition of metabolic activity over time, as measured by formazan absorbance, are shown at bottom. Buffer alone, conditioned media alone, and unconjugated CCD domain (“Collagen”) were used as controls. Cell metabolic activity and viability results after incubation with purified proteins are consistent with those of rAAV administration as shown in FIGS. 8C and 21B. A similar MTT assay in mouse glioma GL261 cells for evaluation of six CCD fusion proteins for any decoy receptor activity is shown in FIG. 15D.

Next, a bispecific CCD fusion protein containing a first heterologous scFv having affinity for a tau protein (α-tau) and a second heterologous scFv having affinity for an amyloid beta peptide 9 (α-Aβ9) was constructed and the binding affinity/avidity of this protein to its ligands was tested in vitro using binding assays and in vivo through IHC imaging and amyloid plaque detection. (Bispecific CCD fusion constructs are sometimes referred to herein as “double warheads.”) A schematic of the hypothesized structure of this fusion protein, following its multimerization into a hexamer in vivo, is shown in FIG. 6A.

First, expression levels of this fusion protein in cell media were detected relative to expression levels of unconjugated α-tau 3A6 scFv and α-Aβ9 scFv (i.e., in the absence of CCD). Results are shown in in FIG. 6C.

The α-tau/α-Aβ9 CCD fusion protein was expressed and then isolated and purified. Next, binding affinity and kinetics of fusion protein were evaluated by ELISA and SPR. ELISA results, shown in in FIG. 6B, indicate that the fusion protein binds its ligands equally as well as the scFv peptides. SPR biolayer interferometry experiments were performed as described above, in two separate stages: first, the fusion protein in solution was allowed to flow over an immobilized biotinylated tau peptide ligand; subsequently, the same fusion protein solution was allowed to flow over immobilized Aβ9 ligand. Results indicate that fusion proteins were able to bind tau (e.g. at the N-terminus) and then bind Aβ9 (at the C-terminus) sequentially. Results are shown in FIG. 6E.

An rAAV2/8 particle encoding the α-tau/α-Aβ9 CCD fusion protein (or nanobody) was administered to the brains of CRDN8 mice and a human Alzheimer's Disease patient. The fusion protein successfully reduced amyloid plaque deposition in the brains relative to administration of unconjugated scFv's in solution (see FIG. 6D). Based on the amyloid plaque reduction observed in this functional in vivo assay, it can be inferred that this monomeric nanobody has enhanced in vivo avidity to Aβ9 and tau than the same scFv's in an unconjugated state.

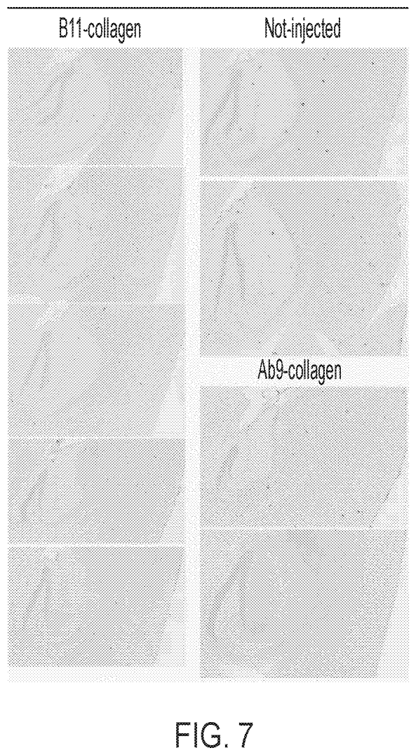

In a confirmatory assay, rAAV administration of the bispecific CCD fusion proteins comprising anti-Aβ9 scFv and B11 scFv targeting VEGFR-2 reduces amyloid plaque depositions in vivo in CRND8 transgenic mice that overexpress amyloid precursor protein (APP). These results are shown in FIG. 7.

These results indicate that the α-tau/α-Aβ9 CCD fusion nanobodies exhibit avidity to ligands in vivo to a degree that is substantially equivalent to the sum of the binding affinities of α-tau scFv and α-Aβ scFv (in the absence of CCD) for the same ligands in vivo.

In conclusion, the creation of a platform for generation of fusion proteins is herein disclosed. Fusion of multiple proteins to the CCD domain dramatically increase levels of secreted fusion protein partners (in some cases the protein goes from undetectable levels when expressed without the domain to a very high level). Fusion to the CCD also may increase the functional avidity of decoy receptors and recombinant antibody fragments.

### Example 5: Glucocerebrosidase and HSV-TK Delivery

A mutated glucocerebrosidase (GBA) gene is implicated in Gaucher disease and is a risk factor in Parkinson's disease. A GBA-CCD fusion protein construct was designed and generated for potential use in therapy of Gaucher disease and Parkinson's disease. A “double warheads” GBA-CCD-antiAβ9 scFv fusion protein was also generated. The α-Aβ9 scFv domain of the construcy could potentially enhance the therapeutic effectiveness of the encoded GBA-CCD fusion protein for Parkinson's disease. GBA activity assay and α-Aβ9 scFv activity ELISA assay were assessed to show whether the GBA and the scFv domains retained their respective activities while conjugated to the CCD domain.

In the GBA activity assay, GBA-CCD and GBA-CCD-Aβ9 scFv constructs were overexpressed in HEK293T cells. Cells were harvested, and the conditioned cell media was collected. Conditioned media, 293T cell lysate and protein purified from media and lysate were prepared in assay buffer (50 mM sodium citrate, 25 mM sodium cholate, 5 mM DTT, pH 6.0). Substrate 4-Methylumbelliferyl β-D-glucopyranoside was added to make a final concentration of 6 mM. After incubation at 37° C. for 20 min, the reaction was stopped by adding stop solution (0.5 M glycine, 0.3 M NaOH, pH 10). Fluorescence emission at 445 nm was used to measure GBA activity.

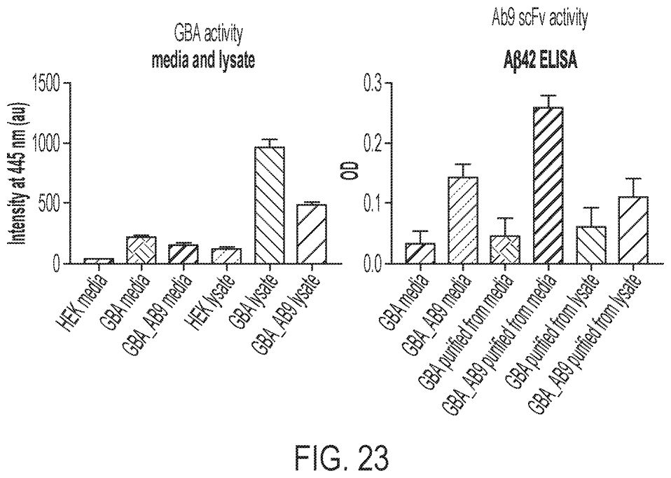

An ELISA-based assay was performed to assess Aβ9 scFv activity. 96-well plates were coated with Aβ42 peptide, and 293T conditioned media (containing the GBA-CCD and GBA-CCD-Aβ9 scFv proteins) and cell lysate were then added and incubated for 6 hours. ELISA was developed with anti-FLAG M2 HRP. (See FIG. 23.)

Herpes Simplex Virus-1 thymidine kinase (HSV-TK) is one of the most well characterized suicide gene used in cancer therapy and in other diseases. HSV-TK is capable of altering antiviral drug ganciclovir into a toxic agent. As a result, HSV-TK expression combined with gancyclovir administration causes death in only cells transduced with and actively expressing HSV-TK. For this reason, the HSV-TK gene is called a “suicide gene”.

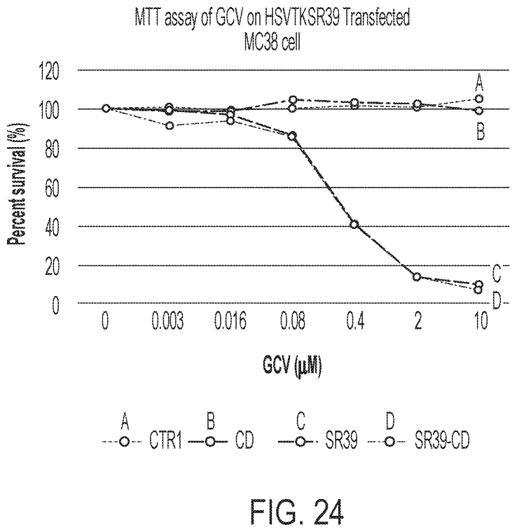

A fusion protein construct of HSV-TK-CCD wild type was designed and generated. In addition, a modified fusion construct encoding HSV-TK-SR39, which has higher efficacy in cancer therapy (published data from others), was generated for comparative testing. To confirm that CCD fusion does not interfere with HSV-TK suicide activity, HSV-TK (wild-type)- and HSV-TK-SR39-CCD fusion protein constructs were transfected into colon adenocarcinoma MC38 cells, and gancyclovir was subsequently added. After a 72 hour incubation, cell survival were measured using an MTT assay (see FIG. 24). The results show that HSV-TK activity was retained while the protein was conjugated to CCD.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms. All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A fusion protein comprising: (i) a C1qTNF protein collagen domain (CCD) and (ii) a first heterologous peptide, wherein the CCD is derived from a C1qTNF of human origin.

2. The fusion protein of claim 1 further comprising (iii) a second heterologous peptide.

3. The fusion protein of claim 1 or 2, wherein the CCD comprises a sequence having at least 90% sequence identity, at least 95% sequence identity, or at least 99% sequence identity to the amino acid sequence of SEQ ID NOs: 1 or 11.

4. The fusion protein of any one of claims 1-3, wherein the CCD comprises a sequence having at least 90% sequence identity, at least 95% sequence identity or at least 99% sequence identity to the amino acid sequence of SEQ ID NOs: 2 or 3.

5. The fusion protein of any one of claims 1-3, wherein the CCD comprises a sequence having at least 90% sequence identity, at least 95% sequence identity or at least 99% sequence identity to the amino acid sequence of any one of SEQ ID NOs: 4-9.

6. The fusion protein of any one of claims 1-4, wherein the fusion protein comprises the structure NH₂-[first heterologous peptide]-[CCD]-COOH, NH₂-[CCD][first heterologous peptide]-COOH, NH₂-[first heterologous peptide]-[CCD]-[second heterologous peptide]-COOH, NH₂-[CCD][first heterologous peptide]-[second heterologous peptide]-COOH, or NH₂-[first heterologous peptide]-[second heterologous peptide]-[CCD]-COOH, wherein each instance of “]-[” indicates the presence of an optional linker sequence.

7. The fusion protein of any one of claims 1-6, wherein the optional linker sequence comprises the amino acid sequence GGGS (SEQ ID NO: 103) or GGGSGGGS (SEQ ID NO: 104).

8. The fusion protein of any one of claims 1-7, wherein the first and second heterologous peptides are secretable human peptides.

9. The fusion protein of any one of claims 1-8, further comprising one or more FLAG or His₆ tags.

10. The fusion protein of any one of claims 1-9, further comprising a secretion signal sequence.

11. The fusion protein of any one of claims 1-10, wherein the fusion protein has bispecificity for two or more ligands.

12. The fusion protein of any one of claims 1-11, wherein at least one of the first or second heterologous peptides is a single-chain variable fragment (scFv).

13. The fusion protein of any one of claims 1-11, wherein at least one of the first or second heterologous peptides is an immune checkpoint modulator.

14. The fusion protein of any one of claims 1-11, wherein at least one of the first or second heterologous peptides is a soluble lymphocyte activation gene (sLag).

15. The fusion protein of any one of claims 1-11, wherein at least one of the first or second heterologous peptides is a growth factor receptor.

16. The fusion protein of any one of claims 1-15, wherein at least one of the first or second heterologous peptides is selected from the group consisting of Lag3, PD-1, WIF1, Icos, NOTCH1, VEGFR1, GBA, HSV-TK, HSV-TK-SR39, and FGFR1.

17. The fusion protein of any one of claims 1-16, wherein at least one of the first or second heterologous peptides is selected from the group consisting of NOTCH4, CTLA4, DLL3, JAG1, B7H3, EGFR, TGFBR1, TGFBR2, HHIP, SFRP1, SFRP2, DKK1, SOSTDC1, CSF1R, TIGIT, TRAIL, TIMP3, HAVCR2, a VEGFR1 domain 2, a VEGFR2 domain 3, and an ectodomain of a soluble toll-like receptor (sTLR).

18. The fusion protein of claim 12, wherein the second heterologous peptide comprises an scFv having affinity for an amyloid beta peptide (α-Aβ).

19. The fusion protein of claim 18, wherein the first heterologous peptide comprises an scFv having affinity for a tau protein (α-tau).

20. The fusion protein of claim 19, wherein the fusion protein comprises the structure NH₂-[α-tau scFv]-[CCD]-[α-Aβ scFv]-COOH, NH₂-[α-tau scFv]-[CCD]-[α-Aβ scFv]-FLAG-COOH, or NH₂-FLAG-[α-tau scFv]-[CCD]-[α-Aβ scFv]-COOH, wherein each instance of “]-[” indicates the presence of an optional linker sequence.

21. The fusion protein of claim 18, wherein the first heterologous peptide comprises GBA.

22. The fusion protein of claim 12, wherein the first heterologous peptide comprises an scFv having affinity for an amyloid beta peptide (Aβ) and the second heterologous peptide comprises an scFv having affinity for a different amyloid beta peptide.

23. The fusion protein of any one of claims 1-22, wherein the fusion protein comprises an amino acid sequence that is at least 85%, at least 90%, at least 95%, at least 98%, at least 99%, or at least 99.5% identical to any one of the amino acid sequences of SEQ ID NOs: 56, 57, 59, 70, 72, 77, 80, 87, 101, 102, or 105-108.

24. The fusion protein of any one of claims 1-23, wherein the fusion protein comprises any one of the amino acid sequences as set forth in SEQ ID NOs: 13-48, 50-96, 101, 102, or 105-108.

25. The fusion protein of any one of claims 1-24, wherein the fusion protein comprises any one of the amino acid sequences as set forth in SEQ ID NOs: 56, 57, 59, 70, 72, 77, 80, 87, 101, 102, or 105-108.

26. The fusion protein of any one of claims 1-25, wherein the fusion protein comprises any one of the amino acid sequences as set forth in SEQ ID NOs: 57, 87, 102, or 105-108.

27. A multimeric fusion protein comprising two or more monomers comprising any one of the fusion proteins of claims 1-26.

28. The multimeric fusion protein of claim 27 comprising three or six monomers comprising any one of the fusion proteins of claims 1-26.

29. The multimeric fusion protein of claim 27 or 28, wherein the monomers are the same.

30. The multimeric fusion protein of claim 27 or 28, wherein the monomers are not the same.

31. The multimeric fusion protein of any one of claims 27-30, wherein the CCDs of the two or more monomers are linked to each other by one or more glycine-cysteine-glycine linker domains or glycine-tyrosine-cysteine linker domains.

32. The multimeric fusion protein of any one of claims 27-31, wherein the two or more monomers are linked to each other by one or more disulfide bridges.

33. The multimeric fusion protein of any one of claims 27-32, wherein the multimeric fusion protein has bispecificity for two or more ligands.

34. The multimeric fusion protein of any one of claims 27-33, wherein the multimeric fusion protein has a binding avidity for two or more ligands in vivo that is substantially equivalent to the sum of the binding affinities of the first and second heterologous peptides for the same ligands in vivo.

35. A fusion protein comprising (i) a merozoite surface protein domain (MSP) and (ii) a first heterologous peptide.

36. The fusion protein of claim 35, further comprising a second heterologous peptide.

37. The fusion protein of claim 35 or 36, wherein the MSP comprises an amino acid sequence as set forth in SEQ ID NO: 49.

38. The fusion protein of any one of claims 35-37, wherein the fusion protein comprises the structure NH₂-[first heterologous peptide]-[MSP]-COOH, NH₂-[MSP]-[first heterologous peptide]-COOH, NH₂-[first heterologous peptide]-[MSP]-[second heterologous peptide]-COOH, NH₂-[MSP]-[first heterologous peptide]-[second heterologous peptide]-COOH, or NH₂-[first heterologous peptide]-[second heterologous peptide]-[MSP]-COOH, wherein each instance of “]-[” indicates the presence of an optional linker sequence.

39. The fusion protein of any one of claims 35-38, wherein the fusion protein comprises any one of the amino acid sequences as set forth in SEQ ID NOs: 97-100.

40. A polynucleotide encoding the fusion protein of any one of claims 1-26 or claims 35-39.

41. An expression vector comprising the polynucleotide of claim 40.

42. The vector of claim 41, wherein the vector comprises a heterologous promoter driving expression of the polynucleotide.

43. The vector of claim 42, wherein the heterologous promoter is active in human brain cells.

44. The vector of claim 43, wherein the promoter is selected from CMV enhancer/chicken Beta-actin (CAG) promoter, Glial Fibrillary Acidic protein (GFAP) promoter, Microtubule-associated protein 2 (MAP2) promoter or synapsin 1 (SYN) promoter.

45. A pharmaceutical composition comprising the fusion protein of any one of claims 1-26 or the multimeric fusion protein of any one of claims 27-34 and a pharmaceutically acceptable excipient.

46. A pharmaceutical composition comprising the vector of any one of claims 41-43 and a pharmaceutically acceptable excipient.

47. A nanoparticle comprising the composition of claim 46.

48. A recombinant adeno-associated viral (rAAV) particle comprising the polynucleotide of claim 36 or the vector of any one of claims 41-43.

49. The rAAV particle of claim 48, comprising a rAAV2/8 or a rAAV2/1 particle.

50. A method of treating a subject having or at risk of developing a disease, disorder, or condition, the method comprising:
administering to the subject the fusion protein of any one of claims 1-39, the pharmaceutical composition of claim 45 or 46, the nanoparticle of claim 47, or the rAAV particle of claim 48 or 49.

51. The method of claim 50, wherein the subject has been diagnosed with a disease, disorder, or condition.

52. The method of claim 51, wherein the disease, disorder, or condition is Alzheimer's Disease or another neurodegenerative disorder, a cancer, or an autoimmune disease.

53. The method of claims 50-52, wherein the subject is a human.

54. A method of determining a concentration of an analyte in a cell, tissue or organ, comprising:
administering to the cell, tissue or organ of a subject the fusion protein of any one of claims 1-39, the pharmaceutical composition of claim 45 or 46, the nanoparticle of claim 47, or the rAAV particle of claim 48 or 49; and
measuring a concentration of the analyte, wherein the fusion protein has a binding affinity for the analyte.

55. The method of claim 54, wherein the cell is a mammalian cell.

56. The method of claim 54 or 55, wherein the cell has been isolated from a mammalian subject.

57. The method of claim 56, further comprising adjusting a treatment of the subject in view of the concentration of the analyte.

58. A kit for determining a concentration of an analyte in a cell, tissue or organ, comprising:
the fusion protein of any one of claims 1-39, the pharmaceutical composition of claim 45 or 46, the nanoparticle of claim 47, or the rAAV particle of claim 48 or 49; and
a means for measuring binding affinity between the fusion protein and the analyst selected from equilibrium dialysis, equilibrium binding, gel filtration, ELISA, surface plasmon resonance, and spectroscopy.
